(19) United States
(12) Patent Application Publication
(10) Pub. No.: US 2024/0301465 A1
(43) Pub. Date: Sep. 12, 2024

(54) SYSTEMS AND METHODS FOR ANALYZING BIOLOGICAL SAMPLES

(71) Applicant: Cellanome, Inc., Palo Alto, CA (US)

(72) Inventors: Pier Federico GHERARDINI, Palo Alto, CA (US); Tarun Kumar KHURANA, Palo Alto, CA (US); Ali AGAH, Palo Alto, CA (US); Yir-Shyuan WU, Palo Alto, CA (US); Filiz Gorpe YASAR, Palo Alto, CA (US)

(21) Appl. No.: 18/534,328

(22) Filed: Dec. 8, 2023

Related U.S. Application Data
(63) Continuation of application No. PCT/US2022/033116, filed on Jun. 10, 2022.
(60) Provisional application No. 63/209,544, filed on Jun. 11, 2021.

(51) Int. Cl. C12Q 1/6806; C12N 15/10; C12Q 1/6874

(52) U.S. Cl. CPC C12Q 1/6806; C12N 15/1096; C12Q 1/6874

(57) ABSTRACT

The invention is directed to methods and systems for analyzing transcriptomes of single cells. In some embodiments, the invention comprises methods for analyzing transcriptomes of a plurality of single cells on separate regions of the same planar surface.

### CROSS-REFERENCE

This is a continuation application of International Application No. PCT/US2022/033116, filed Jun. 10, 2022, which claims the benefit of U.S. Provisional Application No. 63/209,544, filed Jun. 11, 2021, which is herein entirely incorporated by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Analysis of a biological sample is one of the cornerstones of modern medicine. While there have been recent developments advancing the analysis of specific deoxynucleic acid (DNA) molecules, analysis of nucleic acid molecules (e.g. DNA, ribonucleic nucleic acid (RNA)) generated from specific cells or tissue samples remains as a hurdle to overcome for the industry. For example, the analysis of the gene expression profile (e.g., a transcriptome) in a cell based on the sequences and abundance of the sequenced nucleic acid in the sample is still inefficient and labor intensive. The sequencing techniques currently available have drawbacks. For example, sequencing RNA samples require sample preparation methods that first convert RNA into a double-stranded cDNA format prior to sequencing. As such, the preparation of the biological sample comprising RNA for sequencing is often labor intensive. Furthermore, current sequencing techniques are less than optimal in preserving strand-specific information of the original single-stranded RNA molecule after being converted into double stranded cDNA. Preserving strand-specific information can be important for annotation for determining gene expression levels.

The field of cellular analysis would be advanced by the availability of multiplex single-cell analysis methods, especially RNA-seq methods, which provided sensitive and convenient measurements of cellular gene expression.

### SUMMARY

The invention is directed to methods and systems for generating libraries of cDNAs on surfaces. In some embodiments, the invention is directed to methods and systems for analyzing nucleic acid molecules, such as messenger RNAs, from a plurality of cells on a surface of a solid support. In some embodiments, the invention is directed to methods for synthesizing cDNAs from nucleic acid molecules from a plurality of cells disposed on a surface of a solid support. In some embodiments, methods of the invention are directed to determining the transcriptomes of cells disposed on the surface of a solid support. In some embodiments, such methods may comprise (a) providing a solid support, wherein the solid support comprises on a surface one or more nucleic acid molecule capture probes and a plurality of surface primer probes; (b) contacting separate regions of the surface with the one or more nucleic acid molecules of different cells to yield one or more captured nucleic acid molecules in each of the separate regions, wherein captured nucleic acid molecules in different separate regions are from different cells; and (c) synthesizing cDNA molecules from the captured nucleic acid molecules or derivatives thereof, wherein each of the cDNA molecules is coupled to the surface of the solid support and wherein cDNA molecules coupled to different separate regions are from different cells. In some embodiments, cDNA molecules coupled to the surface comprises cDNA molecules covalently bonded to the surface. In some embodiments, such method further includes amplifying the cDNA molecules or derivatives thereof to generate a plurality of sets of amplicons of cDNA molecules or derivates thereof, wherein each set of amplicons is from a different cell. In some embodiments, transcriptomes of the plurality of cells are determined by sequencing the cDNA molecules of the amplicons. In some embodiments, separate regions of the surface of the solid support are determined by hydrogel chambers enclosing each of the plurality of cells. In some embodiments, the step of contacting comprises treating the cells with a lysing agent that releases the one or more nucleic acid molecules. In some embodiments, the one or more nucleic acid molecules are ribonucleic acids (RNAs). In some embodiments, the surface comprises a diffusivity modifier that reduces or blocks diffusion of the one or more nucleic acids away from the cells. In some embodiments, a diffusivity modifier comprises a gel barrier. In some embodiments, a gel barrier modifying diffusivity comprises a hydrogel chamber. In some embodiments, the surface of the solid support comprises cells disposed thereon. In some embodiments, the surface of the solid support comprises a diffusivity modifier. In variations of the above embodiments, the surface of the solid support is a planar surface. In some embodiments, systems and methods of the invention employ an optical system for collecting optical signal from labeled cells and/or molecules on the planar surface.

Aspects provided herein are a method for preparing a set of complementary deoxynucleic acid (cDNA) molecules or derivatives thereof from one or more nucleic acid molecules, the method comprising: providing a solid support, wherein the solid support comprises one or more nucleic acid molecule capture probes and a plurality of surface primer probes; contacting the solid support with the one or more nucleic acid molecules to yield one or more captured nucleic acid molecules; synthesizing a cDNA molecule from the captured nucleic acid molecule or a derivative, wherein the cDNA molecule is coupled to the solid support; inserting an adapter at the 3′ region of the cDNA molecule or a derivative thereof; and amplifying the cDNA molecule or a derivative thereof to generate the set of cDNA molecules or derivates thereof, wherein the set of cDNA molecules or derivates thereof is coupled to the solid support. In some embodiments, synthesizing comprises performing reverse transcription and one or more second strand synthesis reactions. In some embodiments, the set of cDNA molecules or derivates thereof is coupled to the plurality of surface primer probes. In some embodiments, the adapter comprises a sequence configured to permit initiation of a sequencing reaction on a cDNA molecule of the set of cDNA molecules or derivatives thereof. In some embodiments, the set of cDNA molecules or derivatives thereof comprise the adapter. In some embodiments, the method comprises, following the contacting of the solid support with the one or more nucleic acid molecules, contacting the solid support with a moiety configured to inactivate at least a subset of the one or more nucleic acid molecule capture probes. In some embodiments, the subset of the one or more nucleic acid molecule capture probes comprises one or more nucleic acid molecule capture probes that did not capture a nucleic acid molecule. In some embodiments, the moiety configured to inactivate at least the subset of the one or more nucleic acid molecule capture probes comprises an exonuclease. In some embodiments, the one or more second strand synthesis reactions comprise template switch extension, random priming, or both. In some embodiments, prior to inserting the adapter at the 3′ region of the cDNA molecule, the method comprises amplifying the cDNA molecule or a derivative thereof. In some embodiments, prior to inserting the adapter at the 3′ region of the cDNA molecule, the method comprises in-solution primer sequences. In some embodiments, the cDNA molecules are amplified using the in-solution primer sequences. In some embodiments, prior to inserting the adapter at the 3′ region of the cDNA molecule, the method comprises fragmentation of the cDNA molecule or a derivative thereof. In some embodiments, the inserting of the adapter at the 3′ region of the cDNA molecule comprises single-strand ligation. In some embodiments, the inserting of the adapter at the 3′ region of the cDNA molecule comprises tagmentation. In some embodiments, the inserting of the adapter at the 3′ region of the cDNA molecule comprises double-stranded ligation. In some embodiments, at least a subset of the plurality of surface primer probes comprises a blocking agent that blocks an extension reaction on the at least the subset of the plurality of surface primer probes. In some embodiments, prior to amplifying of the cDNA molecule, the plurality of surface primer probes are subjected to the blocking agent to a reaction that unblocks the at least the subset of the plurality of surface primer probes to permit the extension reaction. In some embodiments, the one or more blocking agents comprise one or more 3′ phosphate nucleotides. In some embodiments, the one or more blocking agents comprise a nucleic acid molecule comprising a sequence complementary to at least the subset of the plurality of surface primer probes. In some embodiments, the one or more blocking agents comprise a nucleic acid molecule comprising a sequence partially complementary to at least the subset of the plurality of surface primer probes, a reversible terminator nucleotide and a polymerase, or any derivatives thereof. In some embodiments, the method comprises cleaving or linearizing at least a subset of the set of cDNA molecules or derivatives thereof. In some embodiments, the method comprises blocking the 3′ end of the subset of the set of DNA molecules or derivatives thereof. In some embodiments, the blocking of the 3′ end of the subset of the set of DNA molecules or derivatives thereof comprises contacting the subset of the set of DNA molecules or derivatives thereof with terminal deoxynucleotidyl transferase (TdT). In some embodiments, the blocking of the 3′ end of the subset of the set of DNA molecules or derivatives thereof comprises contacting the subset of the set of DNA molecules or derivatives thereof with an oligonucleotide comprising a sequence complementary to the 3′ end of the subset of the set of DNA molecules. In some embodiments, the blocking of the 3′ end of the subset of the set of DNA molecules or derivatives thereof comprises contacting the subset of the set of DNA molecules or derivatives thereof with a cationic-neutral diblock polypeptide copolymer. In some embodiments, the method comprises sequencing a subset of the cDNA molecules or derivatives thereof in situ on the solid support. In some embodiments, the method comprises eluting at least a subset of the set of cDNA molecules or derivatives thereof from the solid support. In some embodiments, the one or more nucleic acid molecules comprise DNA or ribonucleic nucleic acid (RNA) molecules. In some embodiments, the DNA is fragmented and single-stranded DNA. In some embodiments, the DNA is single-stranded DNA. In some embodiments, the RNA molecules comprise messenger RNA (mRNA) or microRNA (miRNA). In some embodiments, the RNA molecules comprise mRNA. In some embodiments, the one or more nucleic acid molecule capture probes comprise a sequence configured to couple to the one or more nucleic acid molecules. In some embodiments, the sequence configured to couple to the one or more nucleic acid molecules comprises a poly-T sequence, a randomer, a sequence complementary to at least a subset of the one or more nucleic acid molecules, or any combination thereof. In some embodiments, the solid support may comprise a well, a bead, a gel matrix or a fluidic channel. In some embodiments, the one or more nucleic acid molecule capture probes and the plurality of surface primer probes are attached to a planar surface of the solid support. In some embodiments, the fluidic channel comprises a flow cell. In some embodiments, the one or more nucleic acid molecule capture probes comprise one or more tags, wherein a tag comprises a cell-specific or spatial location-specific identifier sequence and optionally a unique molecular identifier (UMI) sequence. In some embodiments, the amplifying comprises solid-supported amplification. In some embodiments, the solid-supported amplification is bridge amplification. In some embodiments, the one or more nucleic acid molecules are derived from a single cell or biological tissue. In some embodiments, the method occurs in a gel matrix, wherein the gel matrix is adjacent to the solid support.

Aspects provide herein are a method for preparing a set of complementary deoxynucleic acid (cDNA) molecules or derivatives thereof from one or more nucleic acid molecules, the method comprising: providing a solid support, wherein the solid support comprises one or more nucleic acid molecule capture probes; contacting the solid support with the one or more nucleic acid molecules to yield one or more captured nucleic acid molecules; synthesizing a cDNA molecule from the captured nucleic acid molecule or a derivative; first amplifying the cDNA molecule or a derivative thereof to generate an amplified cDNA population; inserting an adapter at the 3′ region of the amplified cDNA molecule or a derivative thereof, thereby generating a tagged amplified cDNA population; and performing solid-supported amplification on the tagged amplified cDNA population to generate the set of cDNA molecules or derivatives thereof. In some embodiments, synthesizing comprises performing reverse transcription of a captured RNA template. In some embodiments, the solid support comprises a plurality of surface primer probes. In some embodiments, the cDNA molecule or the derivative thereof, the amplified cDNA population, the tagged amplified cDNA population, the set of cDNA molecules or derivates thereof, or any combination thereof is coupled to the plurality of surface primer probes. In some embodiments, the adapter comprises a sequence configured to permit initiation of a sequencing reaction on a cDNA molecule of the set of cDNA molecules or derivatives thereof. In some embodiments, the set of cDNA molecules or derivatives thereof comprise the adapter. In some embodiments, the method comprises contacting the solid support with a moiety configured to inactivate at least a subset of the one or more nucleic acid molecule capture probes. In some embodiments, the subset of the one or more nucleic acid molecule capture probes comprise one or more nucleic acid molecule capture probes that did not capture a nucleic acid molecule. In some embodiments, the moiety configured to inactivate at least the subset of the one or more nucleic acid molecule capture probes comprises an exonuclease. In some embodiments, the synthesizing comprises performing one or more second strand synthesis reactions comprising the cDNA molecule or a derivative thereof. In some embodiments, the one or more second strand synthesis reactions comprise template switch extension. In some embodiments, the one or more second strand synthesis reactions comprise random priming. In some embodiments, the method comprises fragmentation of the amplified cDNA molecule. In some embodiments, the inserting of the adapter comprises single-strand ligation. In some embodiments, the inserting of the adapter comprises tagmentation. In some embodiments, the inserting of the adapter comprises double-strand ligation. In some embodiments, at least a subset of the plurality of surface primer probes comprises a blocking agent that blocks an extension reaction on the at least the subset of the plurality of surface primer probes. In some embodiments, the method comprises subjecting the blocking agent to a reaction that unblocks at least a subset of the plurality of surface primed probes to permit the extension reaction. In some embodiments, the one or more blocking agents comprise one or more 3′ phosphate nucleotides. In some embodiments, the one or more blocking agents comprise a nucleic acid molecule comprising a sequence complementary to at least the subset of the plurality of surface primer probes. In some embodiments, the one or more blocking agents comprise a nucleic acid molecule comprising a sequence partially complementary to at least the subset of the plurality of surface primer probes, a reversible terminator nucleotide and a polymerase, or any derivatives thereof. In some embodiments, the method comprises cleaving or linearizing at least a subset of the set of cDNA molecules or derivatives thereof. In some embodiments, the method comprises blocking the 3′ end of the subset of the set of DNA molecules or derivatives thereof. In some embodiments, the blocking of the 3′ end of the subset of the set of DNA molecules or derivatives thereof comprises contacting the subset of the set of DNA molecules or derivatives thereof with terminal deoxynucleotidyl transferase (TdT). In some embodiments, the blocking of the 3′ end of the subset of the set of DNA molecules or derivatives thereof comprises contacting the subset of the set of DNA molecules or derivatives thereof with an oligonucleotide comprising a sequence complementary to the 3′ end of the subset of the set of DNA molecules. In some embodiments, the blocking of the 3′ end of the subset of the set of DNA molecules or derivatives thereof comprises contacting the subset of the set of DNA molecules or derivatives thereof with a cationic-neutral diblock polypeptide copolymer. In some embodiments, the method comprises sequencing the at least the subset of the cDNA molecules or derivatives thereof in situ on the solid support. In some embodiments, the method comprises eluting at least a subset of the set of cDNA molecules or derivatives thereof from the solid support. In some embodiments, the one or more nucleic acid molecules comprise DNA or ribonucleic nucleic acid (RNA) molecules. In some embodiments, the DNA is fragmented and single-stranded DNA. In some embodiments, the DNA is single-stranded DNA. In some embodiments, the RNA molecules comprise messenger RNA (mRNA) or microRNA (miRNA). In some embodiments, the RNA molecules comprise mRNA. In some embodiments, the one or more nucleic acid molecule capture probes comprise a sequence configured to couple to the one or more nucleic acid molecules. In some embodiments, the sequence configured to couple to the one or more nucleic acid molecules comprises a poly-T sequence, a randomer, a sequence complementary to at least a subset of the one or more nucleic acid molecules, or any combination thereof. In some embodiments, the solid support comprises a well, a bead, a gel matrix or a fluidic channel. In some embodiments, the fluidic channel is a flow cell. In some embodiments, the solid support is not a bead. In some embodiments, the one or more nucleic acid molecule capture probes comprise one or more tags, wherein a tag comprises a cell-specific or spatial location-specific identifier sequence and optionally a unique molecular identifier (UMI) sequence. In some embodiments, the first amplifying comprises solid-supported amplification. In some embodiments, the first amplifying comprises in-solution primer sequences. In some embodiments, the solid-supported amplification is bridge amplification. In some embodiments, the one or more nucleic acid molecules are derived from a single cell or biological tissue. In some embodiments, the method occurs in a gel matrix, wherein the gel matrix is adjacent to the solid support.

Aspects provided herein are a method for preparing a set of complementary deoxynucleic acid (cDNA) molecules or derivatives thereof from one or more nucleic acid molecules, the method comprising: providing a solid support, wherein the solid support comprises one or more nucleic acid molecule capture probes and a plurality of surface primer probes, wherein at least a subset of the plurality of surface primer probes comprise a template switch moiety; contacting the solid support with the one or more nucleic acid molecules to yield one or more captured nucleic acid molecule; synthesizing a cDNA molecule from the captured nucleic acid molecule or a derivative, wherein the synthesizing comprises performing reverse transcription; inserting an adapter at the 3′ end of the cDNA molecule or a derivative thereof; and amplifying the cDNA molecule or a derivative thereof to generate the set of cDNA molecules or derivates thereof. In some embodiments, the synthesizing comprises performing one or more second strand synthesis reactions comprising the cDNA molecule or a derivative thereof. In some embodiments, the one or more second strand synthesis reactions are mediated by the subset of the plurality of surface primer probes comprising the template switch moiety. In some embodiments, the one or more second strand synthesis reactions comprise template switch extension. In some embodiments, the cDNA molecule or the derivative thereof, the set of cDNA molecules or derivates thereof, or both is coupled to the plurality of surface primer probes. In some embodiments, the adapter comprises a sequence configured to permit initiation of a sequencing reaction on a cDNA molecule of the set of cDNA molecules or derivatives thereof. In some embodiments, the set of cDNA molecules or derivatives thereof comprise the adapter. In some embodiments, the method comprises contacting the solid support with a moiety configured to inactivate at least a subset of the one or more nucleic acid molecule capture probes. In some embodiments, the subset of the one or more nucleic acid molecule capture probes comprise one or more nucleic acid molecule capture probes that did not capture a nucleic acid molecule. In some embodiments, the moiety configured to inactivate at least the subset of the one or more nucleic acid molecule capture probes comprises an exonuclease. In some embodiments, the method comprises amplifying the cDNA molecule or a derivative thereof. In some embodiments, the amplifying comprises in-solution primer sequences. In some embodiments, the inserting of the adapter comprises fragmentation of the cDNA molecule. In some embodiments, the inserting of the adapter comprises single-strand ligation. In some embodiments, the inserting of the adapter comprises tagmentation. In some embodiments, the inserting of the adapter comprises ligation. In some embodiments, at least a subset of the plurality of surface primer probes comprises a blocking agent that blocks an extension reaction on the at least the subset of the plurality of surface primer probes. In some embodiments, the method comprises subjecting the blocking agent to a reaction that unblocks the at least the subset of the plurality of surface primed probes to permit the extension reaction. In some embodiments, the one or more blocking agents comprise one or more 3′ phosphate nucleotides. In some embodiments, the one or more blocking agents comprise a nucleic acid molecule comprising a sequence complementary to at least the subset of the plurality of surface primer probes. In some embodiments, the one or more blocking agents comprise a nucleic acid molecule comprising a sequence partially complementary to at least the subset of the plurality of surface primer probes, a reversible terminator nucleotide and a polymerase, or any derivatives thereof. In some embodiments, the method comprises cleaving or linearizing at least a subset of the set of cDNA molecules or derivatives thereof. In some embodiments, the method comprises blocking the 3′ end of the subset of the set of DNA molecules or derivatives thereof. In some embodiments, the blocking of the 3′ end of the subset of the set of DNA molecules or derivatives thereof comprises contacting the subset of the set of DNA molecules or derivatives thereof with terminal deoxynucleotidyl transferase (TdT). In some embodiments, the blocking of the 3′ end of the subset of the set of DNA molecules or derivatives thereof comprises contacting the subset of the set of DNA molecules or derivatives thereof with an oligonucleotide comprising a sequence complementary to the 3′ end of the subset of the set of DNA molecules. In some embodiments, the blocking of the 3′ end of the subset of the set of DNA molecules or derivatives thereof comprises contacting the subset of the set of DNA molecules or derivatives thereof with a cationic-neutral diblock polypeptide copolymer. In some embodiments, the method comprises sequencing the at least the subset of the cDNA molecules or derivatives thereof in situ on the solid support. In some embodiments, the method comprises eluting at least a subset of the set of cDNA molecules or derivatives thereof from the solid support. In some embodiments, the one or more nucleic acid molecules comprise DNA or ribonucleic nucleic acid (RNA) molecules. In some embodiments, the DNA is fragmented and single-stranded DNA. In some embodiments, the DNA is single-stranded DNA. In some embodiments, the RNA molecules comprise messenger RNA (mRNA) or microRNA (miRNA). In some embodiments, the RNA molecules comprise mRNA. In some embodiments, the one or more nucleic acid molecule capture probes comprise a sequence configured to couple to the one or more nucleic acid molecules. In some embodiments, the sequence configured to couple to the one or more nucleic acid molecules comprises a poly-T sequence, a randomer, a sequence complementary to at least a subset of the one or more nucleic acid molecules, or any combination thereof. In some embodiments, the solid support comprises a well, a bead, a gel matrix or a fluidic channel. In some embodiments, the fluidic channel is a flow cell. In some embodiments, the one or more nucleic acid molecule capture probes comprise one or more tags, wherein a tag comprises a cell-specific or spatial location-specific identifier sequence and optionally a unique molecular identifier (UMI) sequence. In some embodiments, the amplifying comprises solid-supported amplification. In some embodiments, the solid-supported amplification is bridge amplification. In some embodiments, the one or more nucleic acid molecules are derived from a single cell or biological tissue. In some embodiments, the method occurs in or adjacent to a gel matrix, wherein the gel matrix is adjacent to the solid support.

Aspects provided herein is a solid support comprising one or more nucleic acid molecule capture probes and a plurality of surface primer probes, wherein at least a subset of the plurality of surface primer probes comprise a template switch moiety. In some embodiments, the one or more nucleic acid molecule capture probes comprise a sequence configured to couple to one or more nucleic acid molecules. In some embodiments, the sequence configured to couple to the one or more nucleic acid molecules comprises a poly-T sequence, a randomer, a sequence complementary to at least a subset of the one or more nucleic acid molecules, or any combination thereof. In some embodiments, the solid support comprises a well, a bead, a gel matrix or a fluidic channel. In some embodiments, the fluidic channel is a flow cell. In some embodiments, the one or more nucleic acid molecule capture probes comprise one or more tags, wherein a tag comprises a cell-specific or spatial location-specific identifier sequence and optionally a unique molecular identifier (UMI) sequence. In some embodiments, the one or more nucleic acid molecules comprise DNA or ribonucleic nucleic acid (RNA) molecules. In some embodiments, the DNA is fragmented. In some embodiments, the DNA is single-stranded DNA. In some embodiments, the RNA molecules comprise messenger RNA (mRNA) or microRNA (miRNA). In some embodiments, the RNA molecules comprise mRNA. In some embodiments, the solid support comprises a gel matrix, wherein the gel matrix is adjacent to the solid support.

Aspects provided herein are a method for preparing a set of complementary deoxynucleic acid (cDNA) molecules or derivatives thereof from one or more nucleic acid molecules, the method comprising: providing a solid support, wherein the solid support comprises one or more nucleic acid molecule capture probes and a plurality of surface primer probes; contacting the solid support with the one or more nucleic acid molecules to yield one or more captured nucleic acid molecules; synthesizing a cDNA molecule from the captured nucleic acid molecule or a derivative, wherein the synthesizing comprises performing reverse transcription, and wherein the cDNA molecule is coupled to the solid support; inserting an adapter at the 3′ end of the cDNA molecule or a derivative thereof; and amplifying the cDNA molecule or a derivative thereof to generate the set of cDNA molecules or derivates thereof, wherein the set of cDNA molecules or derivates thereof is coupled to the solid support. In some embodiments, the set of cDNA molecules or derivates thereof is coupled to the plurality of surface primer probes. In some embodiments, the adapter comprises a sequence configured to permit initiation of a sequencing reaction on a cDNA molecule of the set of cDNA molecules or derivatives thereof. In some embodiments, the set of cDNA molecules or derivatives thereof comprise the adapter. In some embodiments, the method comprises contacting the solid support with a moiety configured to inactivate at least a subset of the one or more nucleic acid molecule capture probes. In some embodiments, the subset of the one or more nucleic acid molecule capture probes comprise one or more nucleic acid molecule capture probes that did not capture a nucleic acid molecule. In some embodiments, the moiety configured to inactivate at least the subset of the one or more nucleic acid molecule capture probes comprises an exonuclease. In some embodiments, the synthesizing comprises performing one or more second strand synthesis reactions comprising the cDNA molecule or a derivative thereof. In some embodiments, the one or more second strand synthesis reactions comprise template switch extension. In some embodiments, the one or more second strand synthesis reactions comprise random priming. In some embodiments, the method comprises amplifying the cDNA molecule or a derivative thereof. In some embodiments, the amplifying comprises in-solution primer sequences. In some embodiments, the inserting of the adapter comprises fragmentation of the cDNA molecule. In some embodiments, the inserting of the adapter comprises single-strand ligation. In some embodiments, the inserting of the adapter comprises tagmentation. In some embodiments, the inserting of the adapter comprises ligation. In some embodiments, at least a subset of the plurality of surface primer probes comprises a blocking agent that blocks an extension reaction on the at least the subset of the plurality of surface primer probes. In some embodiments, the method comprises subjecting the blocking agent to a reaction that unblocks the at least the subset of the plurality of surface primed probes to permit the extension reaction. In some embodiments, the one or more blocking agents comprise one or more 3′ phosphate nucleotides. In some embodiments, the one or more blocking agents comprise a nucleic acid molecule comprising a sequence complementary to at least the subset of the plurality of surface primer probes. In some embodiments, the one or more blocking agents comprise a nucleic acid molecule comprising a sequence partially complementary to at least the subset of the plurality of surface primer probes, a reversible terminator nucleotide and a polymerase, or any derivatives thereof. In some embodiments, the method comprises cleaving or linearizing at least a subset of the set of cDNA molecules or derivatives thereof. In some embodiments, the method comprises blocking the 3′ end of the subset of the set of DNA molecules or derivatives thereof. In some embodiments, the blocking of the 3′ end of the subset of the set of DNA molecules or derivatives thereof comprises contacting the subset of the set of DNA molecules or derivatives thereof with terminal deoxynucleotidyl transferase (TdT). In some embodiments, the blocking of the 3′ end of the subset of the set of DNA molecules or derivatives thereof comprises contacting the subset of the set of DNA molecules or derivatives thereof with an oligonucleotide comprising a sequence complementary to the 3′ end of the subset of the set of DNA molecules. In some embodiments, the blocking of the 3′ end of the subset of the set of DNA molecules or derivatives thereof comprises contacting the subset of the set of DNA molecules or derivatives thereof with a cationic-neutral diblock polypeptide copolymer. In some embodiments, the method comprises sequencing the at least the subset of the cDNA molecules or derivatives thereof in situ on the solid support. In some embodiments, the method comprises eluting at least a subset of the set of cDNA molecules or derivatives thereof from the solid support. In some embodiments, the one or more nucleic acid molecules comprise DNA or ribonucleic nucleic acid (RNA) molecules. In some embodiments, the DNA is fragmented and single-stranded DNA. In some embodiments, the DNA is single-stranded DNA. In some embodiments, the RNA molecules comprise messenger RNA (mRNA) or microRNA (miRNA). In some embodiments, the RNA molecules comprise mRNA. In some embodiments, the one or more nucleic acid molecule capture probes comprise a sequence configured to couple to the one or more nucleic acid molecules. In some embodiments, the sequence configured to couple to the one or more nucleic acid molecules comprises a poly-T sequence, a randomer, a sequence complementary to at least a subset of the one or more nucleic acid molecules, or any combination thereof. In some embodiments, the solid support comprises a well, a bead, a gel matrix or a fluidic channel. In some embodiments, the fluidic channel is a flow cell. In some embodiments, the solid support is not a bead. In some embodiments, the one or more nucleic acid molecule capture probes comprise one or more tags, wherein a tag comprises a cell-specific or spatial location-specific identifier sequence and optionally a unique molecular identifier (UMI) sequence. In some embodiments, the amplifying comprises solid-supported amplification. In some embodiments, the solid-supported amplification is bridge amplification. In some embodiments, the one or more nucleic acid molecules are derived from a single cell or biological tissue. In some embodiments, the method occurs in a gel matrix, wherein the gel matrix is adjacent to the solid support.

Aspects provided herein include a method for preparing sets of complementary deoxynucleic acid (cDNA) molecules or derivatives thereof from one or more nucleic acid molecules from a plurality of cells, the method comprising: providing a solid support comprising a surface comprising one or more nucleic acid molecule capture probes and a plurality of surface primer probes attached thereto and comprising a plurality of cells disposed thereon; contacting separate regions of the surface with the one or more nucleic acid molecules of different cells of the plurality of cells to yield one or more captured nucleic acid molecules in each of the separate regions, wherein nucleic acid molecules in different separate regions are from different cells of the plurality of cells; and synthesizing cDNA molecules from the captured nucleic acid molecules or derivatives thereof, wherein each of the cDNA molecules is coupled to a surface primer probe of the plurality of surface primer probes and cDNAs coupled to different separate areas are from different cells of the plurality of cells. In some embodiments, the contacting comprises treating the different cells of the plurality of cells with a lysing reagent to release the one or more nucleic acid molecules from the different cells of the plurality of cells. In some embodiments, the method further comprises amplifying the cDNA molecules or derivatives thereof to generate a plurality of sets of amplicons of cDNA molecules or derivates thereof. In some embodiments, transcriptomes of the plurality of cells are determined by sequencing the cDNA molecules of the amplicons. In some embodiments, the cDNA molecules attached to the surface comprise spatial barcodes that encode positions on the surface and further comprising eluting the cDNA molecules from the surface prior to the sequencing. In some embodiments, the contacting the one or more nucleic acid molecules is carried out in the presence of a diffusivity modifier that reduces diffusivities of the one or more nucleic acid molecules. In some embodiments, the surface comprises a gel layer encapsulating the cells disposed thereon or wherein each of the cells disposed on the surface are encapsulated by a separate gel body. In some embodiments, each of the cells disposed on the surface are enclosed by a hydrogel chamber. In some embodiments, the hydrogel chamber comprises an interior area and wherein the contacting further comprises incubating the released one or more nucleic acid molecules at a predetermined temperature so that captured one or more nucleic acid molecules are released and re-captured by capture probes within the interior area. In some embodiments, the interior area of the hydrogel chamber is selected so that the coupled cDNA molecules have an expected nearest neighbor distance of at least 0.25 μm. In some embodiments, the interior area of the hydrogel chamber is selected so that the coupled cDNA molecules have an expected nearest neighbor distance of at least 1 μm. In some embodiments, the interior area of the hydrogel chamber is selected so that the coupled cDNA molecules have an expected nearest neighbor distance of at least 2 μm. In some embodiments, the coupled cDNA molecules have an expected nearest neighbor distance in the range of from 0.5 μm and 5 μm.

Aspects provided herein include a hydrogel chamber disposed on a surface wherein the hydrogel chamber comprises an interior area comprising a substantially uniform distribution of nucleic acid molecules from a single cell. In some embodiments, the nucleic acid molecules are mRNA molecules. In some embodiments, the substantially uniform distribution is a Poisson distribution having an expected nearest neighbor distance between the nucleic acid molecules of 1 μm or greater. In some embodiments, the uniform distribution of the nucleic acid molecules is substantially a Poisson distribution.

Aspects provide herein include a method for preparing a set of complementary deoxynucleic acid (cDNA) molecules or derivatives thereof from one or more nucleic acid molecules, the method comprising: providing a solid support comprising a surface, wherein the surface is not partitioned, and wherein the solid support comprises one or more nucleic acid molecule capture probes and a plurality of surface primer probes; generating a discrete region on the solid support, wherein the discrete region comprises one or more cells unique to the discrete region; extracting one or more ribonucleic acid (“RNA”) molecules from the one or more cells, wherein the one or more RNA molecules are captured by one or more nucleic acid molecule capture probes located in the discrete region, thereby generating one or more captured RNA molecules unique to the discrete region; synthesizing a cDNA molecule from the one or more captured RNA molecules or a derivative thereof, wherein the cDNA molecule is coupled to a surface primer probe of the plurality of surface primer probes located in the discrete region; inserting an adapter at the 3′ region of the cDNA molecule or a derivative thereof; and amplifying the cDNA molecule or a derivative thereof to generate the set of cDNA molecules or derivates thereof, wherein the set of cDNA molecules or derivates thereof is coupled to the solid support. In some embodiments, the discrete region is encompassed by a polymer matrix. In some embodiments, each discrete region comprises a single cell. In some embodiments, the method occurs within the polymer matrix. In some embodiments, the polymer matrix forms a hydrogel. In some embodiments, the polymer matrix is formed from one or more polymer precursors. In some embodiments, the polymer matrix comprises pores that are sized to allow diffusion of a reagent through the polymer matrix, wherein the RNA molecule cannot diffuse through the pores of the polymer matrix. In some embodiments, the solid support comprises one or more discrete regions, and wherein the one or more discrete regions are not in fluidic communication with another discrete region. In some embodiments, the solid support is not a bead. In some embodiments, a surface that is not partitioned comprises a planar surface wherein each point or location on the planar surface is in fluid communication with every other point or location on the planar surface.

### BRIEF DESCRIPTION OF THE DRAWINGS

This patent application contains at least one drawing executed in color. Copies of this patent or patent application with color drawing(s) will be provided by the Office upon request and payment of the necessary fee.

FIG. 1A illustrates a diagram for current techniques for generating a transcriptome, in accordance with some embodiments.

FIG. 1B illustrates a workflow for current techniques for generating a transcriptome, in accordance with some embodiments.

FIG. 2A illustrates a workflow for obtaining an improved transcriptome from in situ and direct generation of sequence-able library from captured mRNA. A surface coating of first and second primers (such as, P5 and P7 primers) is used to generate the library directly on the surface. The amplification step can potentially be done using bridge amplification but could be done through in solution primers. The surface clusters thus generated can either be directly sequenced in situ or the library can be eluted off of surface for sequencing separately.

FIG. 2B illustrates a problem addressed by one embodiment of the invention which relates to analyzing nucleic acid molecules released by adjacent cells on a planar surface.

FIG. 2C illustrates an embodiment for addressing the problem of FIG. 2B using a diffusivity modifier.

FIG. 2D illustrates an embodiment wherein a diffusivity modifier is a gel layer encapsulating cells disposed on a planar surface.

FIGS. 2E-2F illustrates an embodiment wherein diffusivity modifiers are hydrogel chambers enclosing single cells.

FIG. 2G illustrates an embodiment wherein a diffusivity modifier is a hydrogel layer except for cavities, or wells, surrounding cells.

FIG. 2H illustrates a problem related to the distribution of captured nucleic acid molecules adjacent to a cell from which they are released.

FIG. 2I illustrate an embodiment of the invention which addresses the distribution problem of FIG. 2H by employing hydrogel chambers and methods for destabilizing and re-capturing cellular nucleic acid molecules to produce a uniform distribution of captured molecules on the interior surface of a hydrogel chamber.

FIG. 3 illustrates a use of a 3′-phosphate nucleotide to block and unblock the primer as needed in the workflow of obtaining the transcriptome by utilizing the methods and systems described herein. This use is similar to the use as seen in FIG. 2A. However, initially surface P5 and P7 primer probes are blocked (shown as P5* and P7*). When they are going to be used for amplification or library construction, they can then be unblocked using a chemical or enzymatic processes. This decreases unwanted surface hybridization and background.

FIG. 4 illustrates a use of complimentary sequences to keep the primers unavailable by hybridizing to the primers. The primers can be activated or made available by de-hybridization and washing away of the complimentary sequences. Similarly to FIG. 3, instead of blocking surface P5 and P7 primer probes, the P5 and P7 primer probes can be hybridized with complimentary P5′ and P7′ probes on the surface for decreasing unwanted hybridizations. The P5 and P7 primer probes can be unhybridized before surface activation.

FIG. 5 illustrates a use of terminal deoxynucleotidyl transferase (TdT, top panel) or complimentary oligonucleotide (bottom panel) to decrease nucleic acid degradation due to secondary structures. TdT enzyme can block the 3′ end of the cDNA cluster so that if this end folds back onto the cDNA molecule (more specifically on Poly-T capture probe) and form secondary structures, it cannot be extended and creating noisy signals during sequencing, leading to decreasing quality of the sequencing signal. This step can be done after the clustering and cleaving or linearization step. Alternatively, a complimentary oligonucleotide can be used in place of TdT (bottom panel) to prevent self-folding.

FIG. 6 illustrates in situ amplification of the mRNA before fragmentation to improve a workflow for obtaining and increasing the quality of singe cell transcriptome in an off-flow cell workflow (top panel) and on-flow cell workflow (bottom panel).

FIG. 7 illustrates a use of exonuclease to digest the unused and unattached primers on surface or in-solution. For example, unattached capture probes (e.g. the probes with Poly-T) that have not captured any RNA molecules can be digested.

FIG. 8A illustrates a use of primers for template switching. Instead of using free-in-solution template switch on the oligonucleotides, surface primers can be used for template switching. This improvement can increase the efficiency of the template switching process and also avoid formation of concatemers during the template switching process thus increasing the efficiency of mRNA capture for the entire workflow.

FIG. 8B illustrates an embodiment in which barcodes and capture probe are grouped on different surface primers.

FIG. 9 illustrates a combinatorial uses of the improvements described in FIG. 2 (Library Construction on Surface), FIG. 3 (3′ blocking of surface primers for avoiding unwanted hybridization and extension), FIG. 5 (use of TdT for blocking 3′ end of cDNA), and FIG. 7 (use of exonuclease treatment to remove unwanted capture probes) to improve upon the sequencing techniques currently available.

FIG. 10 illustrates a schematic illustration of a portion of a channel disposed in a fluidic device, according to some embodiments.

FIG. 11A illustrates a portion of a system as provided herein including an energy source, according to some embodiments.

FIG. 11B illustrates a polymer matrix being formed around a biological component in a portion of a system as provided herein, according to some embodiments.

FIG. 11C illustrates a method of forming a polymer matrix around a biological component in a system as provided herein, according to some embodiments.

FIG. 12 is a flow chart depicting an embodiment of forming a polymer matrix.

FIG. 13A illustrates a portion of a channel including capture elements in a fluidic device, according to some embodiments.

FIG. 13B illustrates biological components coupled to capture elements on a surface of a portion of a system comprising a channel in a fluidic device, according to some embodiments.

FIG. 13C illustrates polymer matrices disposed around biological components in a system comprising a portion of a channel of a fluidic device, according to some embodiments.

FIG. 14 is a flow chart depicting an embodiment of forming a polymer matrix around a biological component coupled to a surface.

FIG. 15A illustrates a portion of another embodiment of a system comprising a fluidic device including a sealable aperture.

FIG. 15B illustrates a method of trapping biological components in a portion of a system comprising a fluidic device, according to some embodiments.

FIG. 16A illustrates a top view of a schematic of a portion of a system comprising a fluidic device, according to some embodiments.

FIG. 16B illustrates a top view of a schematic of a system comprising a portion of a fluidic device including polymer matrices, according to some embodiments

FIG. 17 illustrates a top view of a schematic of a portion of a system comprising a fluidic device including multiple different reagents, according to some embodiments.

FIG. 18A illustrates a portion of a spatial energy modulating element and a cylindrical polymer matrix, according to some embodiments.

FIG. 18B illustrates a portion of a spatial energy modulating element and polymer matrices in the shape of hollow cylinders, according to some embodiments.

FIG. 19 illustrates a micrograph of polymer matrix compartments encapsulating one or more biological components, according to some embodiments.

FIG. 20A illustrates open compartments formed in a multi-step polymer matrix formation process, according to some embodiments.

FIG. 20B illustrates closed compartments formed in a multi-step polymer matrix formation process, according to some embodiments.

FIG. 21A is a schematic illustration of a portion of a surface of a fluidic device coated with repelling elements, according to some embodiments.

FIG. 21B illustrates a micrograph of biological components captured on a surface using repelling elements, according to some embodiments.

FIG. 21C illustrates a higher magnification micrograph of biological components captured on a surface using repelling elements, according to some embodiments.

FIGS. 22A and 22B illustrate systems for synthesizing hydrogel chambers for use with the invention.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

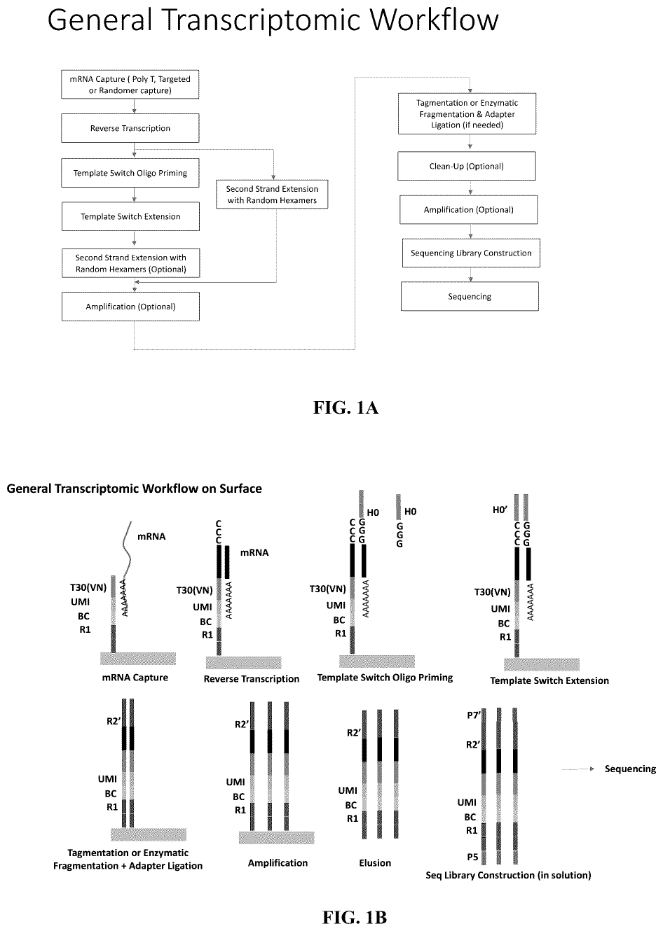

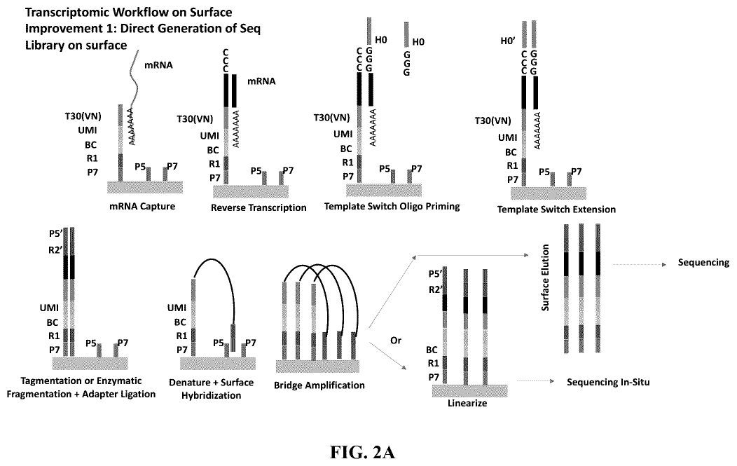

Provided herein are systems and methods, including functionalized solid supports, for generating sequencing-ready nucleic acid molecules (e.g. a set of DNA molecules). The systems and methods described herein provide a set of nucleic acid molecules that can be sequenced in situ on the solid support on which they were generated, or can be eluted off the solid support to be sequenced off-site, where the systems and methods improve upon the sequencing techniques currently available (FIGS. 1A and 1B) with emphasis on second strand synthesis. In some embodiments, the systems and methods can sequence a first strand nucleic acid, a second strand nucleic acid, or both the first and second strand of the nucleic acid of the nucleic acid molecule. In some embodiments, the method comprises preparing a set of complementary deoxynucleic acid (cDNA) molecules or derivatives thereof from one or more nucleic acid molecules by: providing a solid support, wherein the solid support comprises one or more nucleic acid molecule capture probes and a plurality of surface primer probes; contacting the solid support with the one or more nucleic acid molecules to yield one or more captured nucleic acid molecules; synthesizing a cDNA molecule from the captured nucleic acid molecule or a derivative, wherein the synthesizing comprises performing reverse transcription and one or more second strand synthesis reactions, wherein the cDNA molecule is coupled to the solid support; and inserting an adapter at the 3′ region of the cDNA molecule or a derivative thereof; and amplifying the cDNA molecule or a derivative thereof to generate the set of cDNA molecules or derivates thereof, wherein the set of cDNA molecules or derivates thereof is coupled to the solid support. In some embodiments, the method comprises contacting the solid support with a moiety configured to inactivate at least a subset of the one or more nucleic acid molecule capture probes.

In some embodiments, the method comprises inserting an adapter at the 3′ region of the amplified cDNA molecule or a derivative thereof, thereby generating a tagged amplified cDNA population; and performing solid-supported amplification on the tagged amplified cDNA population to generate the set of cDNA molecules or derivatives thereof. In some instances, the subset of the one or more nucleic acid molecule capture probes comprise one or more nucleic acid molecule capture probes that did not capture a nucleic acid molecule. In some aspects, the moiety configured to inactivate at least the subset of the one or more nucleic acid molecule capture probes comprises an exonuclease. In some cases, at least a subset of the plurality of surface primer probes comprises a blocking agent that blocks an extension reaction on the at least the subset of the plurality of surface primer probes. In some embodiments, the method comprises blocking the 3′ end of the subset of the set of DNA molecules or derivatives thereof. In some embodiments, described herein is a method for preparing a set of complementary deoxynucleic acid (cDNA) molecules or derivatives thereof from one or more nucleic acid molecules, the method comprising: providing a solid support, wherein the solid support comprises one or more nucleic acid molecule capture probes and a plurality of surface primer probes, wherein at least a subset of the plurality of surface primer probes comprise a template switch moiety; contacting the solid support with the one or more nucleic acid molecules to yield one or more captured nucleic acid molecule; synthesizing a cDNA molecule from the captured nucleic acid molecule or a derivative, where the synthesizing comprises performing reverse transcription; inserting an adapter at the 3′ end of the cDNA molecule or a derivative thereof; and amplifying the cDNA molecule or a derivative thereof to generate the set of cDNA molecules or derivates thereof.

In some embodiments, described herein is a method for preparing a set of complementary deoxynucleic acid (cDNA) molecules or derivatives thereof from one or more nucleic acid molecules, the method comprising: providing a solid support, wherein the solid support comprises one or more nucleic acid molecule capture probes and a plurality of surface primer probes, wherein at least a subset of the plurality of surface primer probes comprise a template switch moiety; contacting the solid support with the one or more nucleic acid molecules to yield one or more captured nucleic acid molecule; synthesizing a cDNA molecule from the captured nucleic acid molecule or a derivative, wherein the synthesizing comprises performing reverse transcription; inserting an adapter at the 3′ end of the cDNA molecule or a derivative thereof; and amplifying the cDNA molecule or a derivative thereof to generate the set of cDNA molecules or derivates thereof.

In some embodiments described herein is a system comprising a solid support comprising one or more nucleic acid molecule capture probes and a plurality of surface primer probes, where at least a subset of the plurality of surface primer probes comprise a template switch moiety. In some embodiments, described herein is a method utilizing the system for preparing a set of complementary deoxynucleic acid (cDNA) molecules or derivatives thereof from one or more nucleic acid molecules, the method comprising: providing a solid support, wherein the solid support comprises one or more nucleic acid molecule capture probes and a plurality of surface primer probes; contacting the solid support with the one or more nucleic acid molecules to yield one or more captured nucleic acid molecules; synthesizing a cDNA molecule from the captured nucleic acid molecule or a derivative, wherein the synthesizing comprises performing reverse transcription, and wherein the cDNA molecule is coupled to the solid support; inserting an adapter at the 3′ end of the cDNA molecule or a derivative thereof; and amplifying the cDNA molecule or a derivative thereof to generate the set of cDNA molecules or derivates thereof, where the set of cDNA molecules or derivates thereof is coupled to the solid support. In some embodiments, the method utilizing the system comprises contacting the solid support with a moiety configured to inactivate at least a subset of the one or more nucleic acid molecule capture probes. In some instances, the subset of the one or more nucleic acid molecule capture probes comprise one or more nucleic acid molecule capture probes that did not capture a nucleic acid molecule. In some embodiments, the moiety configured to inactivate at least the subset of the one or more nucleic acid molecule capture probes comprises an exonuclease. In some embodiments, the method comprises amplifying the cDNA molecule or a derivative thereof. In some embodiments, the at least a subset of the plurality of surface primer probes comprises a blocking agent that blocks an extension reaction on the at least the subset of the plurality of surface primer probes.

In some embodiments, the systems or methods described herein comprise using a polymer matrix (e.g., a hydrogel matrix) formed adjacent to or around at least of portion of an individual component in a fluidic device described herein. The hydrogel matrix may be selectively generated to surround a component after the system detects the component or hydrogel matrices can be generated according to a predefined pattern in a fluidic device. The hydrogel matrix may allow reagents and smaller entities to pass while retaining the individual component of the biological sample in place. Because one or more individual components can be localized within a fluidic device (e.g., encapsulated) and the localized components be exposed to one or more reagents and/or washing solutions during and/or in between analyses, multiple assays can be performed within the compartments (e.g., simultaneously, substantially simultaneously, serially, etc.). Different assays may be performed in different locations of the fluidic device, for example, to test effects of different treatment conditions. Additionally, because components are not generally mixed and combined, low concentrations of components (e.g., due to dilution) can be prevented. For example, when analyzing genomic material, an amplification step can be avoided due to the preservation of the genetic material in each compartment. By having two or more components within a compartment, interactions between components can be studied as well. The polymer matrix can be degradable “on demand” allowing for controlled localization and release mechanisms. The solutions provided herein can retain spatial information of the components and generate data on a cellular, proteomic, transcriptomic, or genomic level. Since spatial information is retained, the data can be associated (e.g., linked) with phenotypic data. Further, the solutions provided herein can retain spatial information of the components and link data (e.g., phenotypic data) on a cellular, proteomic, transcriptomic, or genomic level.

In some embodiments, the invention is directed to the synthesis in separate regions of a surface sets of surface-attached cDNA molecules. Such surface-attached cDNA molecules are derived from captured nucleic acids, such that cDNA molecules in different separate regions are substantially from different cells. Whenever cells are disposed on the surface randomly, e.g. in a Poisson distribution, there is a trade-off between a concentration (or density) of cells for minimizing overlap of captured molecules and for maximization of throughput, for example, in measuring single-cell transcriptomes. That is, a higher density of cells on the surface means there is a greater likelihood that captured nucleic acids from adjacent cells will overlap and a lower density means fewer transcriptomes are measured. In accordance with aspects of the invention, this trade-off may be affected by the provision of a diffusivity modifier; that is, an agent that reduces or blocks the diffusion of nucleic acid molecules released from cells, thereby promoting capture by capture probes closer to the cell of origin (than would be the case in the absence of a diffusivity modifier). Diffusivity modifiers may be a constituent of a reaction mixture, or medium, comprising cells, such as, soluble polymers, like agarose, poly(ethylene glycol) (PEG), dextran, poly(vinyl) alcohol, poly(vinyl) acetate, polyamide, polysaccharide, poly(lysine), polyacrylamide, poly(ethylene oxide), poly(acrylic acid), or the like. In some embodiments, a diffusivity modifier may be a viscosity modifier, such as, glycerol, hydroxyethyl cellulose, carboxymethyl cellulose, or the like. In some embodiments, a diffusivity modifier may be a gel barrier that encompasses some or all of the cells on the surface, such as, a gel layer on the surface. In other embodiments a diffusivity modifier may be a collection of discontinuous gel barriers that encapsulate or enclose individual cells. In some embodiments, such discontinuous gel barriers comprise single masses, or bodies, of gel material that each encapsulate a single cell, or such discontinuous gel barriers may be a collection of gel chambers, or cages, that comprise polymer matrix walls that enclose single cells, but which may or may not be in contact with the cell. In some embodiments, such gel chambers are hydrogel chambers (as described further herein). In some embodiments, contacting nucleic acid molecules to separate regions of a surface is facilitated by providing a diffusivity modifier in a reaction mixture comprising cells. In other embodiments, such contacting is facilitated by providing a hydrogel chamber for each cell of a plurality.

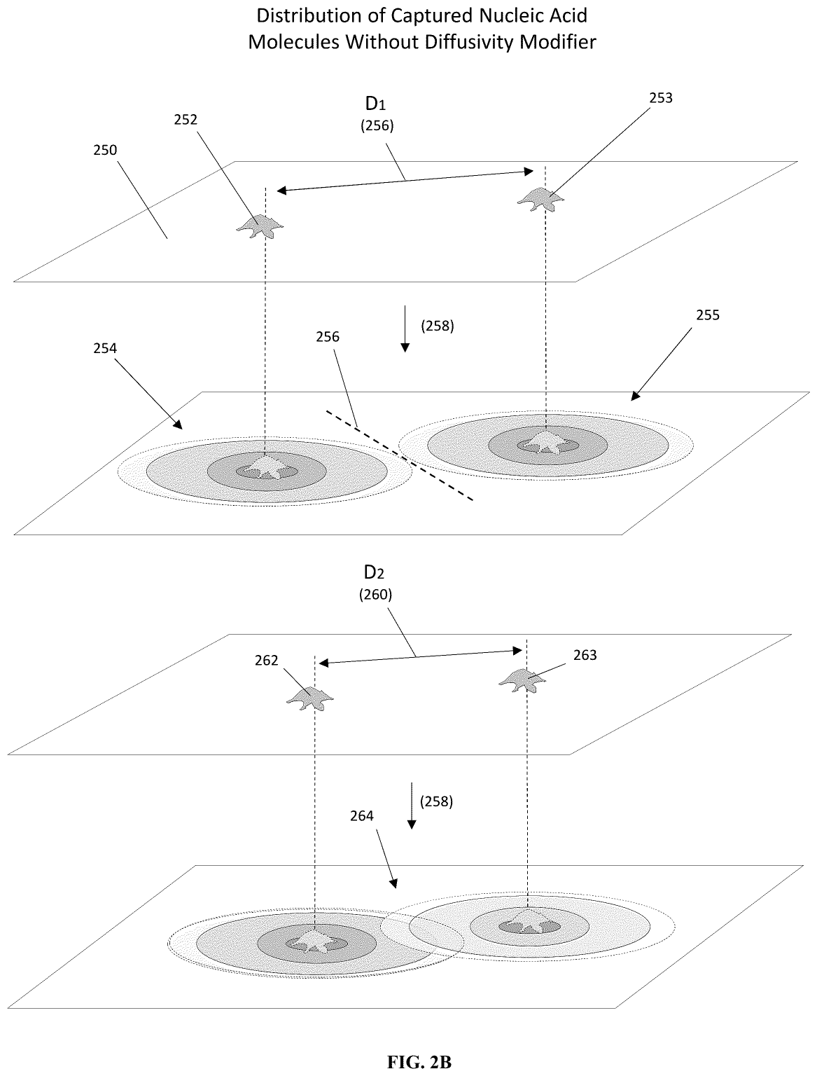

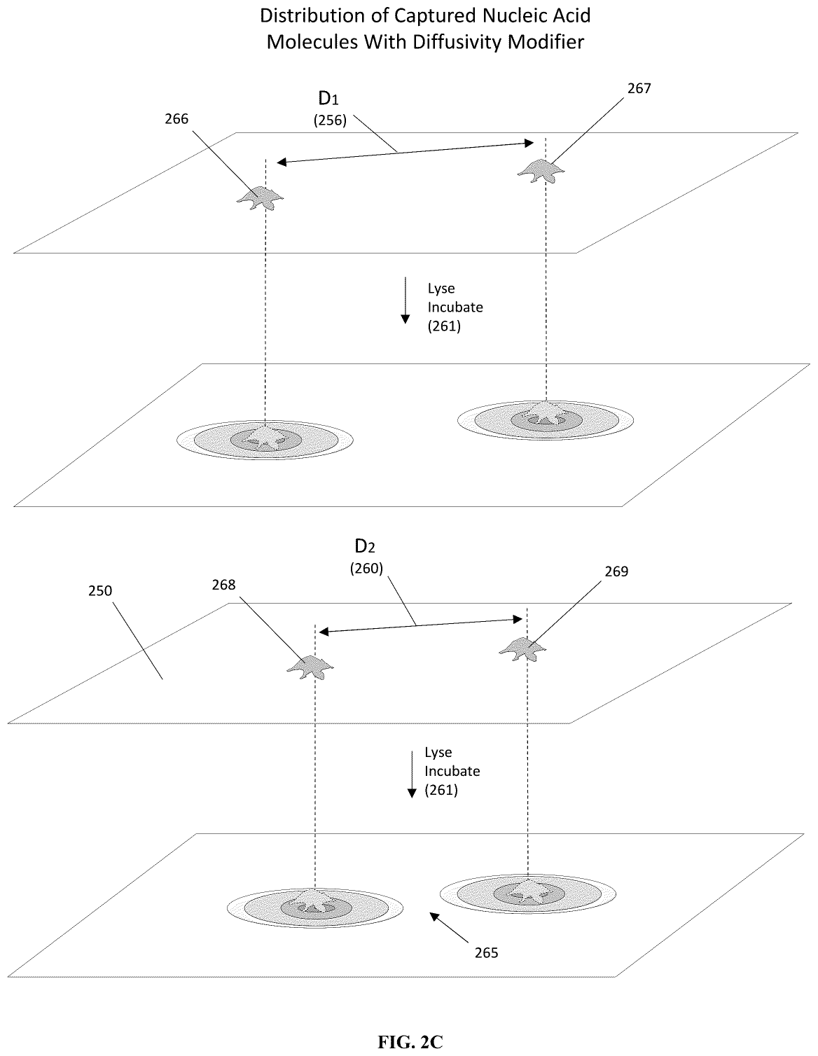

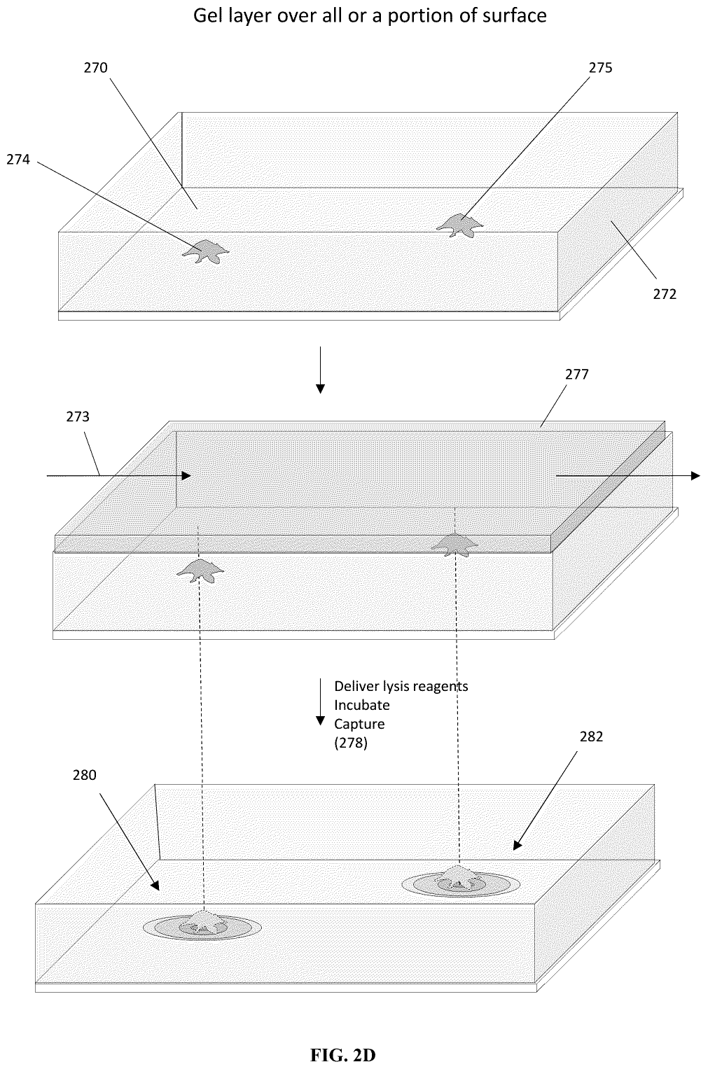

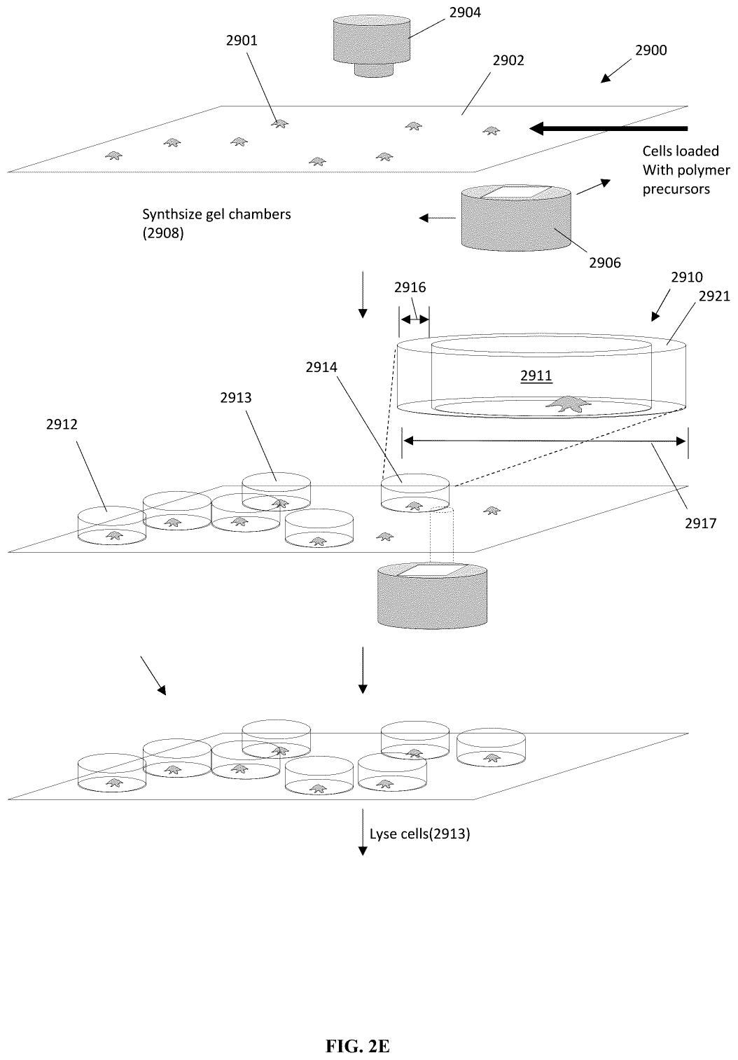

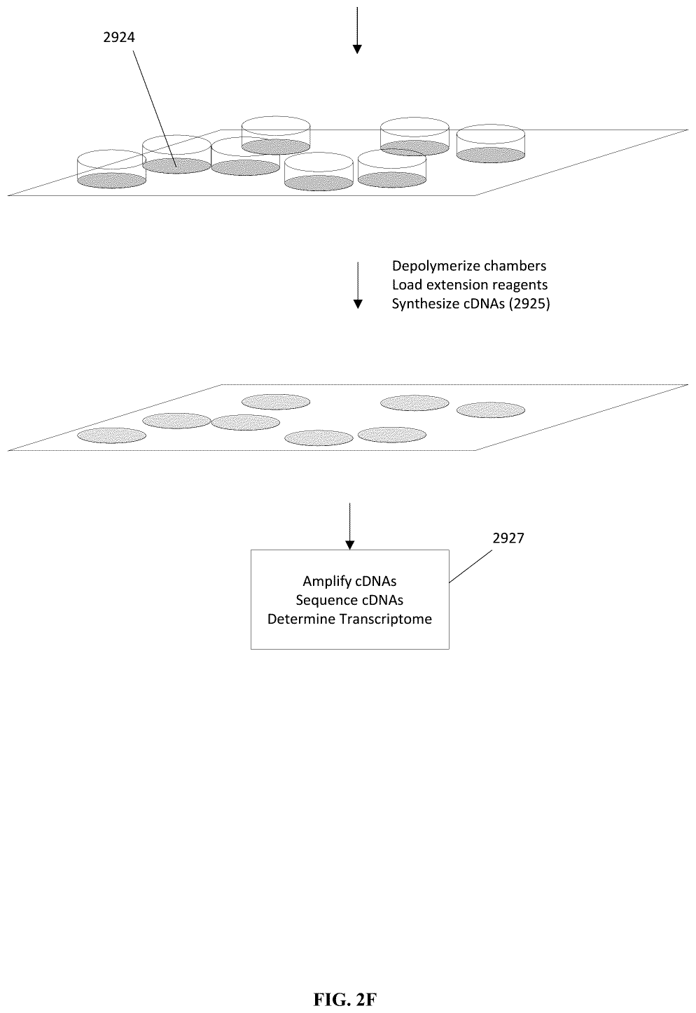

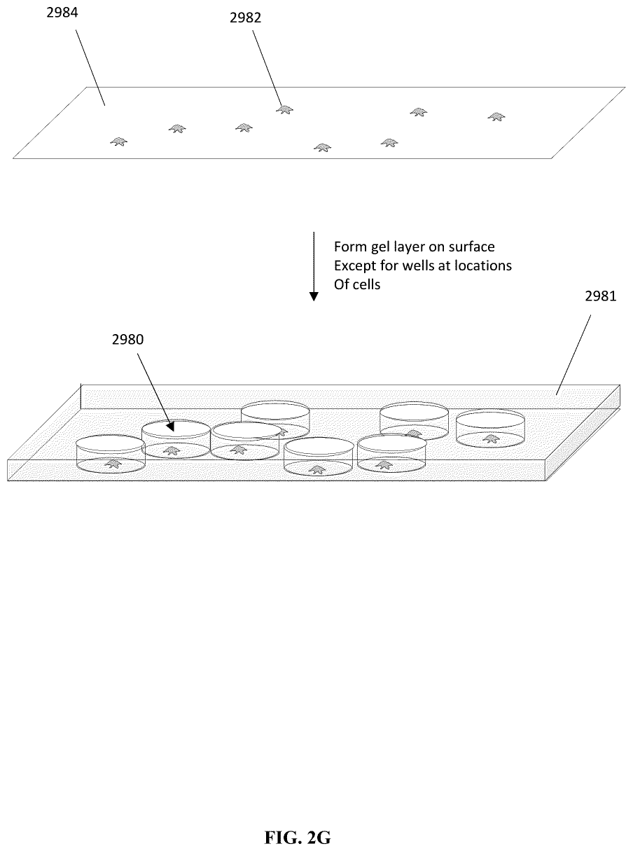

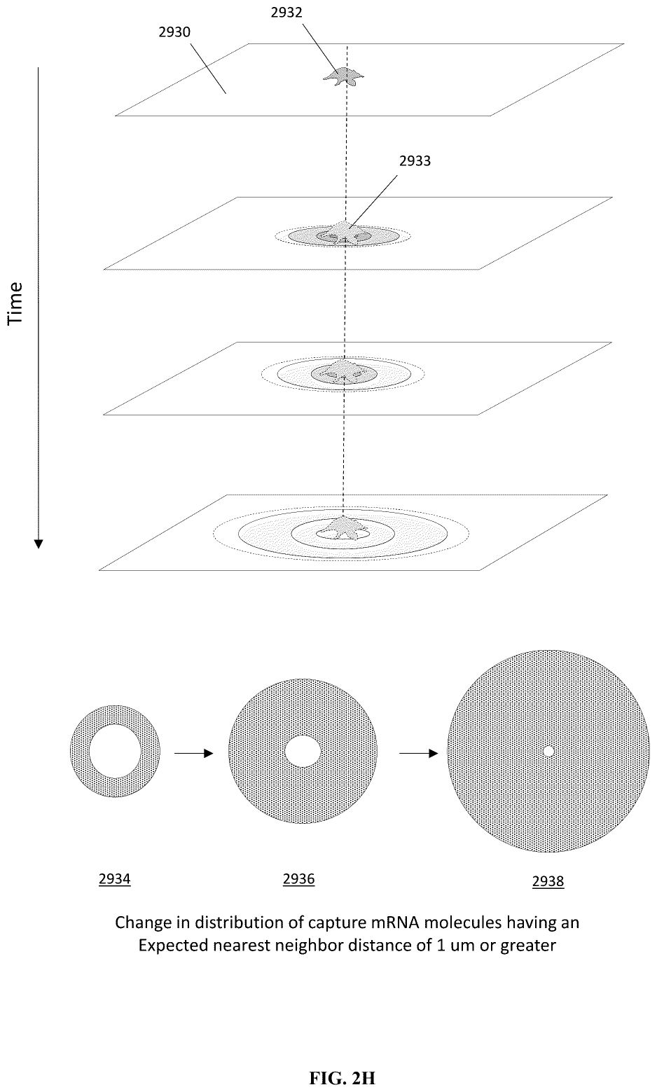

Aspects of the above embodiments are illustrated in FIGS. 2B-2H. The problem addressed by the above embodiments is illustrated in FIG. 2B. In the upper panel, cell (252) and cell (253) are disposed on surface (250) with a distance D₁ (256) between them. Surface (250) comprise one or more nucleic acid molecule capture probes and a plurality of surface primer probes. For example, such capture probes may be designed to capture polyA mRNA, and such surface primer probes may comprise conventional primers, e.g. P5 and P7 primers (or their complements) for surface amplification of captured and reverse transcribed mRNAs. In some embodiments, both sets of capture probes and surface primer probes are covalently attached to surface (250) and uniformly coat surface (250) at predetermined densities. After lysis (258) and release of cellular nucleic acid molecules, such as messenger RNA (mRNA), a portion of the released nucleic acid molecules contacts surface (250) and are captured by capture probes. The rate of capture depends on the concentration of cellular nucleic acid molecules, which after lysis is determined by their diffusion from their cell of origin. Thus, for a given time after lysis, the greatest likelihood of capture is closest to the cell of origin after which such likelihood falls off monotonically as the radial distance increases from the cell of origin. This phenomenon is illustrated for two pairs of cells in FIG. 2B that are different distances from one another. The density distributions of captured nucleic acid molecules ((254) and (255)) from lysed cells (252) and (253), respectively, are shown topologically in gray scale with the highest densities the darkest and the lowest densities the lightest. At a given time after lysis, nucleic acid molecules from a particular cell are predominantly captured by capture probes adjacent to the lysed cell. For cells distance D₁ apart at such time there is no overlap (or minimal overlap) (256) of captured molecules. As illustrated in the lower panel, for cells closer to one another at distance D₂ apart there is significant overlap (264) of captured nucleic acid molecules. In accordance with some embodiments of the invention, the trade-off between density of disposed cells on the surface and, for example, the number of single-cell transcriptome measurements may be improved by carrying out a lysing step in the presence of a diffusivity modifier, which reduces the rate of diffusion of nucleic acid molecules.

As mentioned above, a wide variety of agents are available for reducing the diffusivity of nucleic acid molecules, so that (as illustrated in FIG. 2C) pairs of cells ((266) and (267); (268) and (269)) at the same distances D₁ and D₂ do not produce overlapping captured nucleic acid molecules (265) because the rate of diffusion of the released nucleic acid molecules has been reduced. This allows a greater density of cells to be disposed on a surface (250) for analysis of released cellular molecules, such as cellular nucleic acid molecules.

FIG. 2D illustrates an embodiment employing a diffusivity modifier comprising gel layer (272) that encompassing cells disposed on surface (270). Assay reagents, such as lysis reagents, polymerases, nucleoside triphosphates, primers, and the like, may be delivered to cells by flowing (273) them over gel layer (272) in liquid layer (277) and allowing them to diffuse to cells, e.g. (274) and (275), disposed on surface (270). Gel layer (272) may be formed by disposing cells on surface (270) in a reaction mixture comprising one or more polymer precursors. After disposition, gel layer (272) may be formed by photo-polymerizing the one or more polymer precursor using conventional techniques, as further described below. Polymer precursors and reaction conditions may be selected so that assay reagents may readily diffuse through gel layer (272), while at the same time cellular nucleic acids of interest (e.g. mRNAs comprising greater than 200-300 nucleotides) have reduced diffusivity. After delivery of lysis reagents (278) cells are incubated for a time to permit (i) the lysis reagent to reach the cells, (ii) cell lysis to take place, (iii) cellular nucleic acid molecules to be released, and (iv) released nucleic acid molecules to diffuse away from the cell of origin and/or be capture by capture probes. As illustration by the distributions of captured nucleic acid molecules (280) and (282) from cells (274) and (275), respectively, gel layer (272) ensures that a larger fraction (on average) of captured nucleic acid molecules from different cells are in separate areas of surface (270). In some embodiments, gel layer (272) is a hydrogel layer that is formed by photo-crosslinking polymer precursors.

FIG. 2E-2G illustrate diffusivity modifiers comprising hydrogel chambers. As illustrated in FIG. 2E, cells and polymer precursors are loaded onto surface (2902) which may be part of a channel of a fluidic device (described more fully below). Cells (e.g. 2901) are disposed on surface (2902) and the positions of cells are determined by detector (2904) which are used by a control system to generate instructions for spatial energy modulating element (2906) to produce light beams to synthesize (2908) hydrogel chambers in channel (2900) around single cells, as illustrated for example by hydrogel chambers (2912, 2913 and 2914). Blow-up (2910) illustrates that the solid appearing structures (2912, 2913 and 2914) have interiors (2911) and walls (2921) with predetermine thickness (2916). Likewise, hydrogel chambers have a predetermined shape (e.g. circular with diameter (2917)) and enclose predetermined areas. In the figures, for convenience, chambers are illustrated as standing in isolation without connection with adjacent chambers and as having a cylindrical or annular-like shapes; however, a spatial energy modulating element may synthesize chambers of different shapes and sizes, as is useful for particular applications. In some embodiments, surface (2902) may be part of a flow cell and/or channel in a fluidic device, and hydrogel chambers may be synthesized between surface (2902) and a parallel second surface (not shown in FIG. 2E). As used herein, “channel” means a container capable of holding fluid (which may be static or flowing) and having at least one surface on which cellular assays (such as, transcriptome measurement) may be conducted. In some embodiments, a channel may have a first surface and/or a second surface on which chambers may be synthesized and/or on which cells or assay components may be attached. In addition, in some embodiments, cells or assay components may be attached or capture by capture elements on a polymer matrix wall. As used herein, attributes of a “first surface” (for example, as a surface comprising capture elements) may also apply to a second surface, or as appropriate, a polymer matrix wall (all of which may be interior surfaces of hydrogel chambers). As used herein, a “channel” comprises a solid support comprising a surface, particularly, a planar surface. In some embodiments, a channel may comprise a first surface and a second surface which, for example, may be parallel to one another. In some embodiments, a channel may also constrain a flow of fluid therethrough from an inlet to an outlet. In other embodiments, a channel may comprise a non-flowing volume of fluid that may be removed, replaced or added to by way of an opening or inlet; that is, in some embodiments, a channel of the invention may be a well or a well-like structure. The perpendicular distance between a first surface and a second surface may be in the range of from 10 μm to 500 μm, or in the range of from 50 μm to 250 μm. In some embodiments, the perpendicular distance between a first surface and a second surface may be in the range of from twice the average size of the cells to be analyzed to five times the average size of the cells to be analyzed.

In some embodiments, each hydrogel chamber synthesized on a surface may have the same shape and area, for example, annular-like with an interior area selected from the range of 0.001 to 0.1 mm², or in the range of 0.001 to 1.0 mm². In some embodiments, each hydrogel chamber synthesized has the same shape and area for each different type of cell being assayed. In some embodiments in which mammalian cells are assayed the number of hydrogel chambers synthesized around single cells may be greater than 100; or greater than 1000; or greater than 10,000; or the number may be in the range of from 100 to 100,000; or in the range of from 1000 to 100,000 or in the range of from 1000 to 10⁶.

After hydrogel chambers have been synthesized cells are treated (2913) with a lysing reagent so that cellular nucleic acids, e.g. mRNAs, are released into the interiors of the hydrogel chambers (e.g. 2924) where a portion are capture by capture probes. In some embodiments, after capture the hydrogel chambers may be depolymerize (2925) prior to introduction of extension and/or amplification reagents, e.g. polymerase, dNTPs, primers, and the like, for cDNA synthesis and/or amplification.

FIG. 2G illustrates an embodiment of a diffusivity modifier that comprises a gel layer (2981) as above except that cavies, or wells, (2980) surround cells (2982) disposed on surface (2984). Thus, for example, if cells (2982) are disposed randomly on surface (2984) a random well array if formed around them by selectively polymerizing polymer precursors beyond a given radius from each cell, e.g. by photo-polymerization. As in the case of a solid gel layer, reagents for assays may be delivered to cells by flowing them over the gel layer so that they can enter the wells.

As illustrated in FIG. 2H, after treating cell (2932) on surface (2930) with a lysing reagent, cellular nucleic acid molecules, such as mRNAs, are captured by adjacent capture probes as such molecules diffuse away from their cell of origin (2932). (Lighter colored cells with dashed outline (e.g. 2933) represent lysed cells.) In addition, initially captured molecules will dissociate, diffuse some distance and be re-captured, after which the process is repeated. In some embodiments, captured mRNAs are converted into cDNAs which, in turn, are surface amplified (e.g. by bridge PCR) to form clusters, or clonal populations, of cDNAs which are sequenced, e.g. using conventional sequencing-by-synthesis techniques. For successful sequencing with such approaches, the number of clonal cDNAs per cluster must be large enough to produce detectable signals, and the spacing of the clusters must be large enough to avoid overlaps between clusters, which would degrade signals due to different cDNAs in the overlapped regions. In regard to the latter parameter, for conventional sequencing-by-synthesis techniques, randomly distributed cDNAs for surface amplification may have expected nearest neighbor distances in the range of from 0.25 to 5.0 μm, or in the range of from 1.0 to 5.0 m. In some embodiments, randomly distributed cDNAs for surface amplification, such as bridge amplification, may have an expected nearest neighbor distance of 0.5 m or greater, or an expected nearest neighbor distance of 1.0 m or greater, an expected nearest neighbor distance of 2.0 m or greater. In some embodiments, randomly distributed cDNAs (or captured mRNAs) on a surface are distributed substantially as a Poisson distribution. Returning to FIG. 2H, as cellular nucleic acid molecules spread out on surface (2930) the region adjacent to the cell of origin that have captured mRNAs within the desired ranges may be qualitatively plotted as shown in the lower panel. At the time of lysis (2934) because of high concentration, captured mRNAs adjacent to the cell have expected nearest neighbor distances too low for acceptable cluster formation. As the diffusion of the mRNA progresses and initially captured mRNA de-hybridizes and is re-captured (2936), the expected nearest neighbor distance increases in the region adjacent to the cell of origin. This process continues (2938) until an equilibrium is reached. In some embodiments, in which hydrogel chambers are synthesized that prevent diffusion of mRNA molecules through its polymer matrix walls, the size of hydrogel chamber (and hence its interior area) may be selected so that as the distribution of captured mRNAs approaches an equilibrium random distribution within a hydrogel chamber in which the expected nearest neighbor distance between captured RNAs (or cDNAs) approaches a value within the desired range. Thus, as illustrated in FIG. 2I, cells (e.g. 2944) are disposed on surface (2946) and hydrogel chamber (2948) is synthesized, after which cell (2944) is lysed to release mRNAs (for example) which are captured by capture probes on surface (2946). After an equilibrium distribution of captured mRNAs is reached, cDNAs are synthesized that have expected nearest neighbor distances within a desired range. For example, if cell (2944) is a mammalian cell with about 2×10⁵ mRNA molecules that are completely released into hydrogel chamber (2948), the captured mRNAs will have an expected nearest neighbor distance of about 1 m when the interior area (2950) of hydrogel chamber (2948) is about 2.8×10⁵ μm² (e.g., area of circle with radius of 300 m), e.g. Pielou, Introduction to Mathematical Ecology (Wiley-Interscience, 1969). In some embodiments, the number of mRNAs remaining in the interior of a hydrogel chamber may be controlled by controlling the permeability (or porosity) of the polymer matrix walls of the hydrogel chamber. For example, polymer matrix wall porosity may be selected so that the number of mRNAs retained may have a molecular weight above a predetermined size. In some embodiments, the rate at which cellular nucleic acids approach an equilibrium distribution in the interior surface of a hydrogel chamber may be increased by introducing agents that destabilize duplex formation, e.g. heat, low salt buffers, chaotropic agents, or the like.

In some embodiments, the above methods may be implemented by the following steps: (a) providing a solid support comprising a surface comprising one or more nucleic acid molecule capture probes and a plurality of surface primer probes attached thereto and comprising a plurality of cells disposed thereon; (b) contacting in the presence of a diffusivity modifier separate regions of the surface with the one or more nucleic acid molecules of different cells to yield one or more captured nucleic acid molecules in each of the separate regions, wherein nucleic acid molecules in different separate regions are from different cells; and (c) synthesizing cDNA molecules from the captured nucleic acid molecules or derivatives thereof, wherein each of the cDNA molecules is coupled to the surface of the solid support and cDNAs couple to different separate areas are from different cells. In some embodiments, cDNAs are “coupled” to the surface by extending a capture probe in a polymerase reaction (such as, a reverse transcriptase extension reaction) with a captured cellular nucleic acid molecule as a template. In some embodiments, contacting comprises treating said cells with a lysing reagent to release the one or more nucleic acid molecules, e.g. mRNAs, from said cells. In some embodiments, the above method further comprises amplifying the cDNA molecules or derivatives thereof to generate a plurality of sets of amplicons of cDNA molecules or derivates thereof. In some embodiments, transcriptomes of the plurality of cells are determined by sequencing the cDNA molecules of the amplicons. In some embodiments, the cDNA molecules attached to the surface comprise spatial barcodes that encode positions on the surface and the method further comprises eluting the cDNA molecules from the surface prior to sequencing. In some embodiments, the surface comprises one or more gel layers, such as, one or more hydrogel layers, that encapsulate the cells disposed thereon. In some embodiments a single hydrogel layer encapsulates substantially all of the cells disposed on the surface. In some embodiments, cells disposed on the surface are encapsulated by separate gel layers or gel bodies.

In some embodiments, the diffusivity modifier comprises hydrogel chambers enclosing cells disposed on the surface. In some embodiments, each cell disposed on the surface is enclosed by a separate hydrogel chamber. In some embodiments, the porosity of the polymer matrix walls of the hydrogel chambers is selected so that cellular nucleic acid molecules having molecular weights within a predetermined size range are effectively prevented from diffusing through such polymer matrix walls. In some embodiments, wherein mRNAs are captured, such size range comprises mRNA molecules greater than 100 ribonucleotides, or greater than 200 ribonucleotides, or greater than 300 ribonucleotides, or greater than 400 ribonucleotides, or greater than 500 ribonucleotides. In some embodiments, after lysing cells hydrogel chambers are incubated at an elevated temperature for a time interval to destabilize duplexes formed between released cellular nucleic acid molecules and their capture probes, so that released cellular nucleic acid molecules become randomly distributed among capture probes of the surface interior to the hydrogel chambers. Such elevated temperature may be in the range of from 25° C. to 95° C., or such elevated temperature may be from 10° C. to 60° C. above room temperature. In some embodiments, such time interval may be in the range of from 30 seconds to 20 minutes, or from 30 seconds to 5 minutes, or from 30 seconds to 2 minutes. In some embodiments, after lysing cells hydrogel chambers are treated with a duplex destabilizing reagent, such as a low salt buffer, for a time interval to destabilize duplexes formed between released cellular nucleic acid molecules and capture probes. After the time interval, such low salt buffer is replaced by a buffer in which stable duplexes form. In some embodiments, the interior area of said hydrogel chamber is selected so that the coupled cDNA molecules (from which clusters are generated) have an expected nearest neighbor distance greater than 1 μm, or in the range of from 0.25 μm and 5 μm, or in the range of from 1 μm and 3 μm.

In some embodiments, the invention comprises a composition of matter comprising a hydrogel chamber disposed on a surface wherein the hydrogel chamber comprises an interior area comprising a random distribution of nucleic acid molecules from a single cell. In some embodiments, the random distribution of nucleic acid molecules is a uniform distribution in that the probability of a given number of nucleic acid molecules being attached within a given sub-area of an interior area depends only on the size of the sub-area. In some embodiments, such uniform distribution is substantially a Poisson distribution. “Substantially a Poisson distribution” as used herein means that the probability that the actual distribution was generated by a Poisson process is greater than 50 percent, or greater than 70 percent, or greater than 90 percent. In some embodiments, uniformly distributed nucleic acid molecules are mRNAs. In some embodiments, uniformly distributed nucleic acid molecules are cDNAs. In some embodiments, such uniform distribution of mRNA molecules or cDNA molecules has an expected nearest neighbor distance between mRNAs or cDNAs of 1 μm or greater.

In some embodiments, hydrogel chambers for use in the invention may be synthesized by the following steps: (a) providing a fluidic device comprising (i) one or more channels each comprising a first surface, (ii) a spatial energy modulating element in optical communication with each first surface, and (iii) a detector that identifies positions of cells in each channel based on one or more optical signals therefrom; (b) loading each channel with cells and one or more polymer precursors so that the cells are disposed on the first surfaces; and (c) synthesizing one or more chambers in each channel, each chamber enclosing one or more cells by projecting light into each channel with the spatial energy modulating element such that the projected light causes cross-linking of the one or more polymer precursors to form polymer matrix walls of the chambers, wherein the positions of the synthesized chambers are determined in each channel by the positions of the cells enclosed thereby identified by the detector. In some embodiments, the method further comprises (i) loading the channel(s) with a lysing reagent so that messenger RNAs of cells are released and captured by the capture probes in the interior of the hydrogel chambers, (ii) loading the channel(s) with reverse transcription reagents to copy the captured messenger RNAs to produce complementary DNAs, and (iii) sequencing the complementary DNAs.

In some embodiments, the polymer matrix walls of the hydrogel chambers are permeable to molecules having a molecular weight less than 3×10⁶ Daltons and are impermeable to molecules having a molecular weight greater than 3×10⁶ Daltons. In some embodiments, the polymer matrix walls of the hydrogel chambers are permeable to molecules having a molecular weight less than 3×10⁵ Daltons and are impermeable to molecules having a molecular weight greater than 3×10⁵ Daltons. In some embodiments, the polymer matrix walls of the hydrogel chambers are permeable to molecules having a molecular weight less than 3×10⁴ Daltons and are impermeable to molecules having a molecular weight greater than 3×10⁴ Daltons. In some embodiments, the polymer matrix walls of the hydrogel chambers are permeable to molecules having a molecular weight less than 3×10³ Daltons and are impermeable to molecules having a molecular weight greater than 3×10³ Daltons.

### Generating A Tagged Set Of Nucleic Acid Molecules For Sequencing

Descried herein are methods for generating a tagged nucleic acid for sequencing. In some embodiments, the tagged nucleic acid is synthesized from one or more nucleic acid molecule captured on a solid support. Non-limiting examples of the solid support include gel matrix or fluidic channel. In some embodiments, the fluidic channel is flow cell. In some embodiments, the solid support is not a bead. In some embodiments, the solid support is contacted or encapsulated with a gel matrix such as a hydrogel. In some embodiments, the solid support is the gel matrix. In some embodiments, the solid support is the encapsulating gel matrix. In some embodiments, the captured nucleic acid molecule is RNA. In some embodiments, the captured nucleic acid molecule (e.g., a mRNA) is washed or degraded off the solid support. In some embodiments, a first strand nucleic acid is tagged and is synthesized from the captured nucleic acid, while a second strand tagged nucleic acid is synthesized from the first strand nucleic acid after the captured nucleic acid molecule is washed or degraded off the solid support. In some embodiments, the method comprises preparing a set of complementary deoxynucleic acid (cDNA) molecules or derivatives thereof from one or more nucleic acid molecules. In some embodiments, the one or more nucleic acid molecules are contacted to a solid support comprising one or more nucleic acid molecule capture probes and a plurality of surface primer probes. In some embodiments, the one or more nucleic acid molecules are captured by the capture probe. In some embodiments, a first strand nucleic acid comprising a cDNA molecule is synthesized from the captured nucleic acid molecule or a derivative, wherein the synthesizing comprises performing reverse transcription, and wherein the cDNA molecule is coupled to the solid support. In some embodiments, cDNA is tagged (e.g., with a barcode such as unique molecule index or UMI). In some embodiments, the cDNA is tagged with a tag comprising a cell-specific or spatial location-specific identifier sequence and optionally an UMI sequence. In some embodiments, an adapter can be inserted or added at the 3′ end of the cDNA molecule or a derivative thereof. In some embodiments, the cDNA molecule or a derivative thereof can be further amplified or sequenced. In some embodiments, the adapter inserted at the 3′ end of the cDNA molecule can serve as an initiation for the sequencing. In some embodiments, the method comprises synthesizing one or more second strands of the cDNA molecule or a derivative thereof. In some embodiments, the one or more second strand synthesis reactions comprise template switch extension. In some embodiments, wherein the one or more second strand synthesis reactions comprise random priming. In some embodiments, the method comprises synthesizing the cDNA library by single-strand ligation. In some embodiments, the cDNA library synthesis comprises tagmentation. In some embodiments, the cDNA library synthesis comprises fragmentation followed by adapter ligation. In some embodiments, the cDNA can be cleaved or linearized. In some embodiments, the cDNA is cleaved or linearized after amplification. In some embodiments, the cDNA is synthesized by solid-supported amplification. In some embodiments, the solid-supported amplification is bridge amplification.

In some embodiments, the one or more nucleic acids contacted with the solid support and serve as template for cDNA synthesis can be DNA or RNA. In some cases, the DNA is singled-stranded. In some embodiments, the DNA is genomic DNA. In some embodiments, the DNA is cell free DNA. In some embodiments, the RNA is ncRNA, nmRNA, sRNA, smnRNA, tRNA, sRNA, mRNA, pcRNA, rRNA, 5S rRNA, 5.8S rRNA, SSU rRNA, LSU rRNA, NoRC RNA, pRNA, 6S RNA, SsrS RNA, aRNA, asRNA, asmiRNA, cis-NAT, crRNA, tracrRNA, CRISPR RNA, DD RNA, diRNA, dsRNA, endo-siRNA, exRNA, gRNA, hc-siRNA, hcsiRNA, hnRNA, RNAi, lincRNA, lncRNA, miRNA, mrpRNA, nat-siRNA, natsiRNA, OxyS RNA, piRNA, qiRNA, rasiRNA, RNase MRP, RNase P, scaRNA, scnRNA, scRNA, scRNA, SgrS RNA, shRNA, siRNA, SL RNA, SmY RNA, snoRNA, snRNA, snRNP, SRP RNA, ssRNA, stRNA, tasiRNA, tmRNA, uRNA, vRNA, vtRNA, Xist RNA, Y RNA, NATs, pre-mRNA, circRNA, msRNA, or cfRNA. In some embodiments, the RNA is mRNA. In some embodiments, the RNA is microRNA (miRNA).

### Probe Inactivation

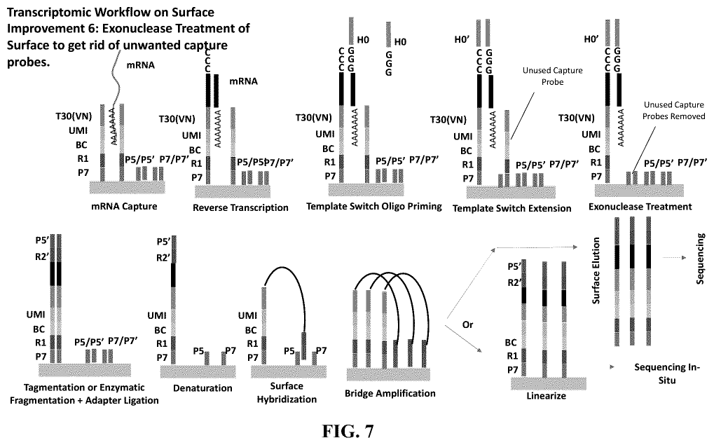

In some embodiments, the method comprises at least a subset of the one or more nucleic acid molecule capture probes can be inactivated. In some embodiments, the at least a subset of the one or more nucleic acid molecule capture probes can be inactivated (FIG. 7). In some embodiments, the at least a subset of the one or more nucleic acid molecule capture probes can be inactivated before contacting with the one or more nucleic acid molecules. In some embodiments, the at least a subset of the one or more nucleic acid molecule capture probes can be inactivated after contacting with the one or more nucleic acid molecules. In some embodiments, the at least a subset of the one or more nucleic acid molecule capture probes can be inactivated by treating the nucleic acid molecule capture probes with a nuclease. In some embodiments, the at least a subset of the one or more primer probes can be inactivated before contacting with the one or more nucleic acid molecules. In some embodiments, the at least a subset of the one or more primer probes can be inactivated after contacting with the one or more nucleic acid molecules. In some embodiments, the at least a subset of the one or more primer probes can be inactivated by treating the primer probes with a nuclease. In some cases, the nuclease can be an endonuclease. In some aspects the nuclease is an exonuclease. Non-limiting examples of nuclease for inactivating the one or more nucleic acid molecule capture probes include S1 nuclease, P1 nuclease, N. crassa nucleases, Mycelia, Conidia, BAL 31 nucleases, U. maydis nucleases, Nuclease Bh1, Aspergillus nuclease, Physarum nuclease, SP nuclease, Mung bean nuclease, Wheat chloroplast nuclease, Nuclease I, Pea seeds nuclease, Tobacco nuclease I, Alfalfa seedling nucleases, SK nuclease, Hen liver nuclease, Rat liver nuclei nuclease, or Mouse mitochondria nuclease.

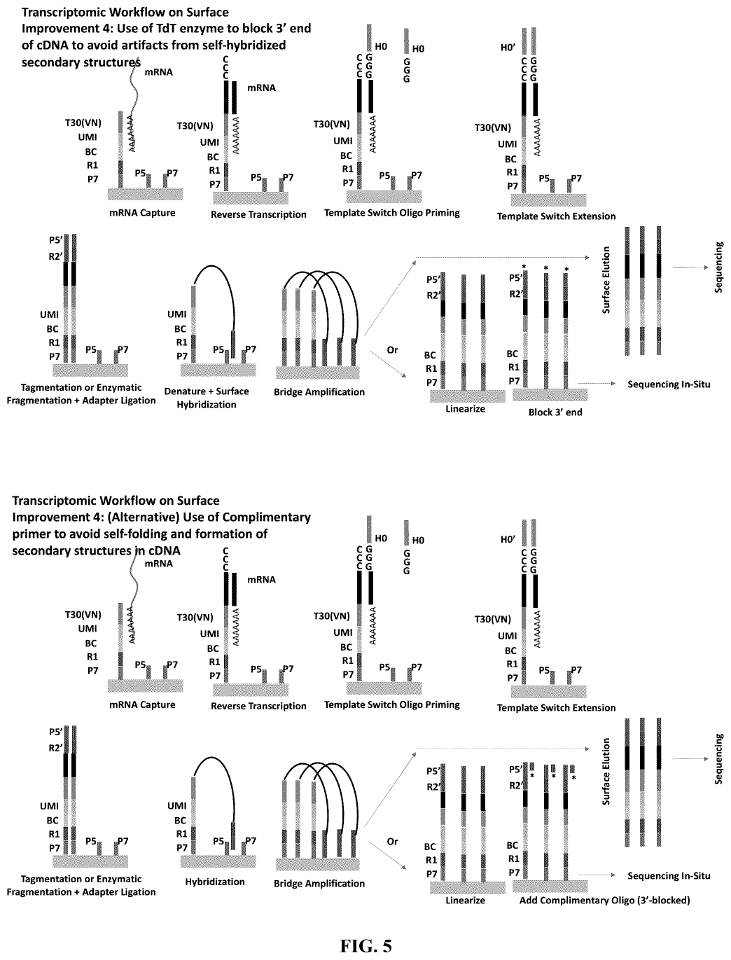

In some embodiments, the one or more nucleic acid molecule capture probes or the primer probes can be inactivated by a moiety that is not a nuclease (FIG. 5). In some embodiments, the one or more nucleic acid molecule capture probes or the primer probes can be inactivated by a moiety comprising TdT enzyme. In some embodiments, the one or more nucleic acid molecule capture probes or the primer probes can be inactivated by hybridizing with complementary oligonucleotides. In some embodiments, the one or more nucleic acid molecule capture probes or the primer probes can be inactivated by hybridizing the one or more nucleic acid molecule capture probes or the primer probes with at least partially complementary oligonucleotides and incorporating a reversible terminator nucleotide with a polymerase. In some embodiments, the one or more nucleic acid molecule capture probes or the primer probes can be inactivated by contacting with a moiety that attaches Phosphate to 3′ end of the oligonucleotide. In some embodiments, the one or more nucleic acid molecule capture probes or the primer probes can be inactivated by contacting with a moiety comprising a cationic-neutral diblock polypeptide copolymer. In some embodiments, the inactivating of the probes decreases self-folding of the probes. In some embodiments, the inactivating of the probes decreases background signal associated with the probes not contacted with one or more nucleic acids or cDNA synthesized from the with one or more nucleic acids.

### Generating cDNAs in Solution

Described herein, in some embodiments, are methods for synthesizing or sequencing the cDNA in solution. In some embodiments, the cDNA molecule or a derivative thereof is synthesized while the one or more nucleic acid molecules are attached to the solid support (e.g., attached with contacting with the one or more nucleic acid molecule capture probes). In some embodiments, a first strand cDNA is synthesized while the one or more nucleic acid molecules are attached to the solid support. In some embodiments, a second strand cDNA is synthesized while the one or more nucleic acid molecules are attached to the solid support. In some embodiments, the synthesized cDNA (both first and second strand) is eluted off the solid support prior to sequencing via contacting with the in-solution primers. In some embodiments, the cDNA molecule is contacted or encapsulated by the gel matrix (e.g. the hydrogel) before being eluted off the solid support. In some embodiments, the cDNA molecule is contacted or encapsulated by the gel matrix (e.g. the hydrogel) after being eluted off the solid support. In some embodiments, the cDNA molecule is suspended in aqueous buffer after being eluted off the solid support.

In some embodiments, the one or more nucleic acids are contacted and captured by the nucleic acid molecule capture probes. Upon contacting with the nucleic acid molecule capture probes, a first strand tagged cDNA sequence can be synthesized. In some embodiments, the captured nucleic acid molecule (e.g., a mRNA) is washed or degraded off the solid support. In some embodiments, the first strand can be eluted off the solid support before the synthesis of the second strand tagged cDNA sequence. In some embodiments, the first and second strand can be eluted off the solid support before sequencing of the cDNA.

In some embodiments, the cDNA molecule or a derivative thereof is synthesized while the one or more nucleic acid molecules are not attached to solid support. In some embodiments, the cDNA molecule or a derivative thereof is synthesized while the one or more nucleic acid molecules are encapsulated in gel matrix such as the hydrogel described herein. In some embodiments, the cDNA molecule or a derivative thereof is synthesized after the one or more nucleic acid molecules are eluted off the solid support. In some embodiments, a first strand cDNA is synthesized after the one or more nucleic acid molecules eluted off the solid support. In some embodiments, a second strand cDNA is synthesized after the one or more nucleic acid molecules are eluted off the solid support. In some embodiments, the cDNA is synthesized by contacting with in-solution primer sequences. In some embodiments, the cDNA synthesized via the use of the in-solution primer sequences is fragmented. In some embodiments, the cDNA synthesized via the use of the in-solution primer sequences comprises tagmentation.

### Blocking Primer Probes

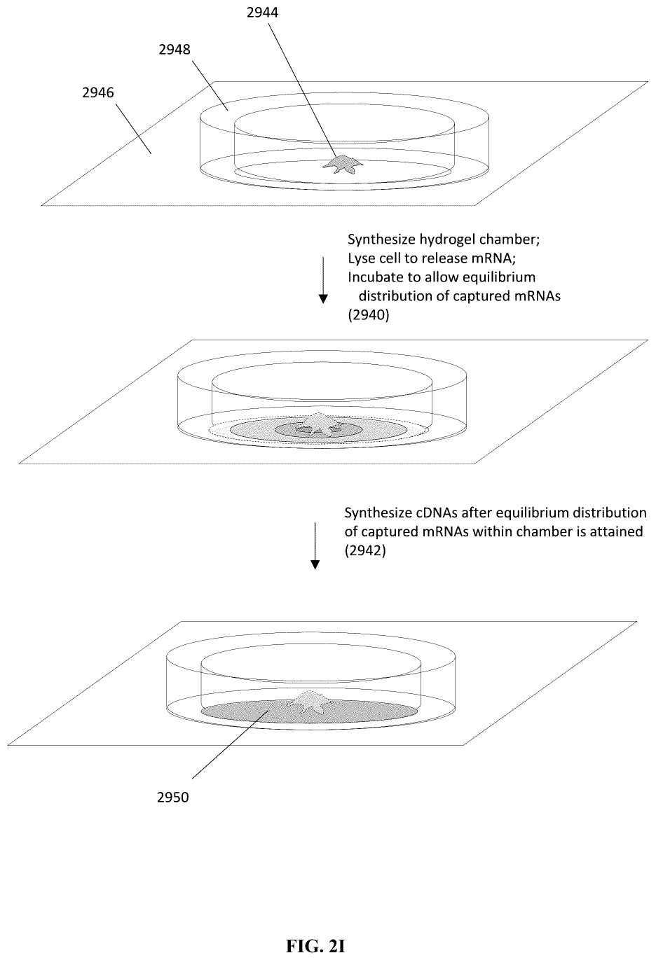

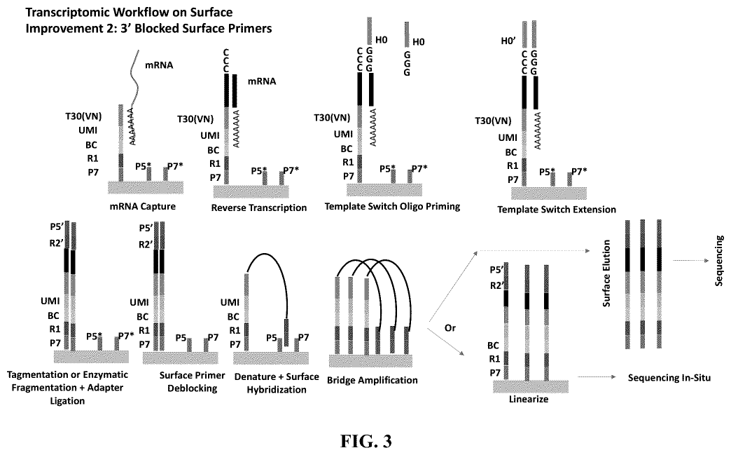

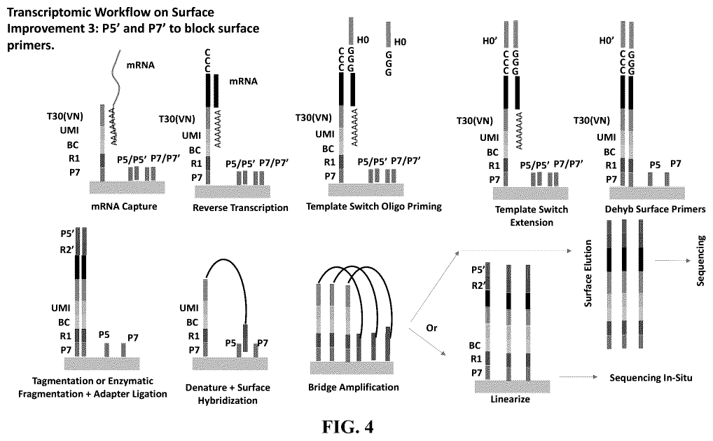

Described herein, in some embodiments, are methods for blocking the surface primer probes by contacting the surface primer probes with the blocking moiety described herein. In some embodiments, the surface primer probes can be blocked by contacting and hybridizing with oligonucleotides comprising nucleic acid sequences that are complementary to the surface primer probes. In some embodiments, the surface primer probes can be blocked by contacting with a blocking agent comprising one or more 3′ phosphate nucleotides. FIG. 3 illustrates a non-limiting example of using a 3′-phosphate nucleotide to block and unblock the surface primer as needed in the workflow of obtaining the transcriptome described herein. This use is similar to the use shown in FIG. 2. However, initially surface P5 and P7 primer probes are blocked (shown as P5* and P7*). When they are going to be used for amplification or library construction, they can then be unblocked using a chemical or enzymatic processes. This decreases unwanted surface hybridization and background signals. FIG. 4 illustrates another example of blocking and unblocking surface primers, where oligonucleotides comprising complimentary sequences to the surface primers can keep the surface primers unavailable by hybridizing to the surface primers. The surface primers can be activated or made available by de-hybridization and washing away of the complimentary sequences. Similarly to FIG. 3, instead of blocking surface P5 and P7 primer probes, the P5 and P7 primer probes can be hybridized with complimentary P5′ and P7′ probes on the surface for decreasing unwanted hybridizations. The P5 and P7 primer probes can be unhybridized before surface activation.

In some embodiments, the method comprises preparing a set of cDNA molecules or derivatives thereof from one or more nucleic acid molecules. In some embodiments, the method comprises contacting a solid support described herein with the one or more nucleic acid molecules to yield one or more captured nucleic acid molecules. In some embodiments, a first strand cDNA molecule is synthesized from the captured nucleic acid molecule. In some cases, a second strand cDNA molecule is synthesized from the first strand. In some embodiments, an adapter is inserted at the 3′ end of the cDNA molecule (first or second strand) or a derivative thereof. In some embodiments, the cDNA molecule is contacted with at least a subset of plurality of surface primer probes for initiation of sequencing reaction for sequencing the cDNA molecule. In some aspects, the cDNA molecules or derivates thereof is coupled to the solid support prior to amplification. In some aspects, the cDNA molecules or derivates thereof is eluted off the solid support prior to amplification. In some embodiments, the surface primer probes comprises a blocking agent that blocks an extension reaction on the at least the subset of the plurality of surface primer probes. In some embodiments, prior to the amplification of the cDNA molecule, the blocking agent is removed to unblock the plurality of surface primer probes to permit the extension reaction.

In some embodiments, the one or more blocking agents comprise one or more 3′ phosphate nucleotides. In some embodiments, the blocking agent comprises an oligonucleotide comprising a sequence complementary to at least the subset of the plurality of surface primer probes. In some embodiments, the one or more blocking agents comprise a nucleic acid molecule comprising a sequence partially complementary to at least the subset of the plurality of surface primer probes, a reversible terminator nucleotide and a polymerase, or any derivatives thereof. In some embodiments, the plurality of surface primer probes is blocked by treating the plurality of surface primer probes with TdT. In some embodiments, the blocking of the 3′ end of the subset of the plurality of surface primer probes comprises contacting the plurality of surface primer probes with a cationic-neutral diblock polypeptide copolymer.

### Generating Tagged Second Strands Of Nucleic Acids For Sequencing

Described herein are methods for generating a second strand of nucleic acid (e.g., a second strand of cDNA molecule) for sequencing. FIG. 2 illustrates a non-limiting example for obtaining an improved transcriptome from in situ and direct generation of sequence-able library from captured mRNA and a tagged second strand of nucleic acid (e.g., a second strand of cDNA molecule). A surface coating of P5 and P7 primer probes can be used to generate the library directly on the surface of the solid support. The amplification can be done using bridge amplification but can also be done through in solution primers. Bridge amplification can comprise amplification using primer probes coated on the surface of the solid support. The primer probes can be attached at the 5′ ends by a flexible linker. At the conclusion of the amplification, each clonal cluster comprises several copies of a single member of the cDNA or the one or more nucleic acid molecules. In some embodiments, the amplification performed through in-solution primers can include using of the emulsion PCR. In one embodiment, one of the PCR primers can be tethered to the surface (5′-attached) of the solid support and the other primer can be in solution. In some cases, the solid support comprises more than one primers, where the primers can target or hybridize with one or more of the nucleic acid molecules or the cDNA molecules.

The surface clusters generated can either be directly sequenced in situ, or the library can be eluted off of surface for sequencing separately. In some embodiments, the method comprises preparing a set of cDNA molecules or derivatives thereof from one or more nucleic acid molecules. In some instances, the method comprises providing a solid support comprising one or more nucleic acid molecule capture probes and a plurality of surface primer probes. In some cases, the method comprises contacting the solid support with the one or more nucleic acid molecules to yield one or more captured nucleic acid molecules. In some embodiments, the cDNA molecule is synthesized from the captured nucleic acid molecule, where the synthesizing of the cDNA can occur when the cDNA or the one or more nucleic acid molecules are attached to the solid support. In some embodiments, the synthesizing of the cDNA can occur when the cDNA or the one or more nucleic acid molecules are eluted off the solid support. In some embodiments, an adapter can be inserted at the 3′ region of the cDNA molecule or a derivative thereof. In some embodiments, the amplification of the cDNA occurs when the set of cDNA molecules or derivates thereof is coupled to the solid support.

In some embodiments, the set of cDNA molecules or derivates thereof is coupled to the plurality of surface primer probes. In some embodiments, the adapter inserted at the 3′ region of the cDNA molecule comprises a sequence configured to permit initiation of a sequencing reaction on a cDNA molecule of the set of cDNA molecules or derivatives thereof. In some embodiments, the one or more nucleic acid molecule capture probes can be inactivated or blocked by any one of the moieties described herein. In some embodiments, the one or more nucleic acid molecule capture probes can be inactivated by any one of the nuclease described herein. In some embodiments, nuclease is an exonuclease. FIG. 7 illustrates a use of exonuclease to digest the unused and unattached primers on surface or in-solution. For example, unattached capture probes (e.g., Poly-T) that have not captured any RNA molecules can be digested.

In some embodiments, the one or more second strand synthesis reactions comprise template switch extension, random priming, or both. In some embodiments, the amplification of cDNA molecule or a derivative thereof occurs when the cDNA is attached to the solid support. In some embodiments, the amplification of cDNA molecule or a derivative thereof occurs when the cDNA is eluted off the solid support. In some embodiments, the amplification of cDNA molecule comprises contacting the cDNA molecule with in-solution primer sequences. In some embodiments, the cDNA comprises fragmentation. In some embodiments, the adapter inserted into the cDNA comprises a sequence for tagmentation. In some embodiments, the adapter is inserted into the cDNA by single-strand ligation. In some embodiments, the adapter is inserted into the cDNA by double-strand ligation.

In some embodiments, the at least a subset of the plurality of surface primer probes comprises a blocking agent that blocks an extension reaction on the at least the subset of the plurality of surface primer probes. In some embodiments, the method comprises subjecting the blocking agent to a reaction that unblocks the at least the subset of the plurality of surface primer probes to permit the extension reaction. In some embodiments, the one or more blocking agents comprise one or more 3′ phosphate nucleotides. In some embodiments, the one or more blocking agents comprise a nucleic acid molecule comprising a sequence complementary to at least the subset of the plurality of surface primer probes. In some embodiments, the one or more blocking agents comprise a nucleic acid molecule comprising a sequence partially complementary to at least the subset of the plurality of surface primer probes, a reversible terminator nucleotide and a polymerase, or any derivatives thereof. FIG. 3 illustrates a non-limiting example of using a 3′-phosphate nucleotide to block and unblock the surface primer as needed in the workflow of obtaining the transcriptome described herein. This use is similar to the use shown in FIG. 2. However, initially surface P5 and P7 primer probes are blocked (shown as P5* and P7*). When they are going to be used for amplification or library construction, they can then be unblocked using a chemical or enzymatic processes. This decreases unwanted surface hybridization and background. FIG. 4 illustrates another example of blocking and unblocking surface primers, where oligonucleotides comprising complimentary sequences to the surface primers can keep the surface primers unavailable by hybridizing to the surface primers. The surface primers can be activated or made available by de-hybridization and washing away of the complimentary sequences. Similarly to FIG. 3, instead of blocking surface P5 and P7 primer probes, the P5 and P7 primer probes can be hybridized with complimentary P5′ and P7′ probes on the surface for decreasing unwanted hybridizations. The P5 and P7 primer probes can be unhybridized before surface activation.

In some embodiments, the method comprises blocking of the 3′ end of the subset of the set of DNA molecules or derivatives thereof by contacting the subset of the set of DNA molecules or derivatives thereof with terminal deoxynucleotidyl transferase (TdT). In other cases, the method comprises blocking of the 3′ end of the subset of the set of DNA molecules or derivatives thereof with contacting the subset of the set of DNA molecules or derivatives thereof with an oligonucleotide comprising a sequence complementary to the 3′ end of the subset of the set of DNA molecules. FIG. 5 illustrates a use of terminal deoxynucleotidyl transferase (TdT, top panel) or complimentary oligonucleotide (bottom panel) to decrease nucleic acid degradation due to secondary structures. TdT enzyme can block the 3′ end of the cDNA cluster so that if this end folds back onto the cDNA molecule (more specifically on Poly-T capture probe) and form secondary structures, it cannot be extended and create noisy signals during sequencing, leading to decreasing quality of the sequencing signal. This step can be done after the clustering and cleaving or linearization step. Alternatively, a complimentary oligonucleotide can be used in place of TdT to prevent self-folding. In some embodiments, the method comprises blocking of the 3′ end of the subset of the set of DNA molecules or derivatives thereof by contacting the subset of the set of DNA molecules or derivatives thereof with a cationic-neutral diblock polypeptide copolymer.

In some embodiments, the one or more nucleic acid molecule capture probes comprise a sequence configured to couple to the one or more nucleic acid molecules. In some aspects, the sequence configured to couple to the one or more nucleic acid molecules comprises a poly-T sequence, a randomer, a sequence complementary to at least a subset of the one or more nucleic acid molecules, or any combination thereof. The method of any one of the preceding claims, wherein the one or more nucleic acid molecule capture probes comprise one or more tags, where a tag comprises a cell-specific or spatial location-specific identifier sequence and optionally a unique molecular identifier (UMI) sequence. In some embodiments, the synthesis or amplification of the cDNA occurs when the one or more nucleic acid molecules or the cDNA molecules are attached to the solid support. In some embodiments, the synthesis or amplification of the cDNA occurs when the one or more nucleic acid molecules or the cDNA molecules are eluted off the solid support. In some embodiments, the synthesis or amplification of the cDNA occurs when the one or more nucleic acid molecules or the cDNA molecules are contacted or encapsulated with gel matrix described herein. In some embodiments, the synthesis or amplification of the cDNA occurs when the one or more nucleic acid molecules or the cDNA molecules are eluted off the solid support and contacted or encapsulated with gel matrix.

### Template Switching Oligonucleotides

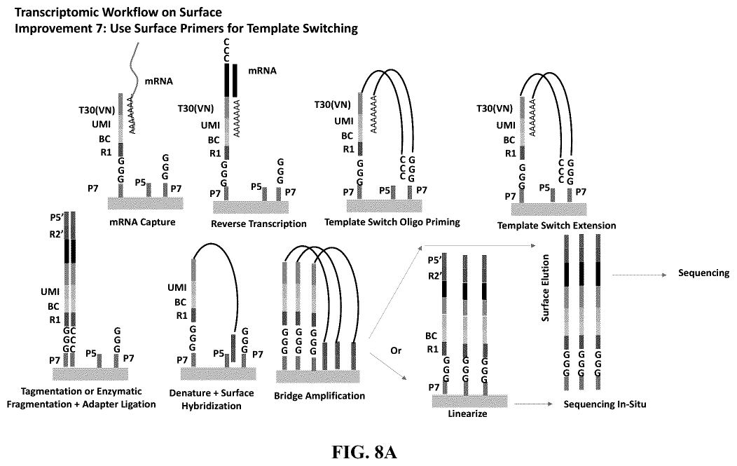

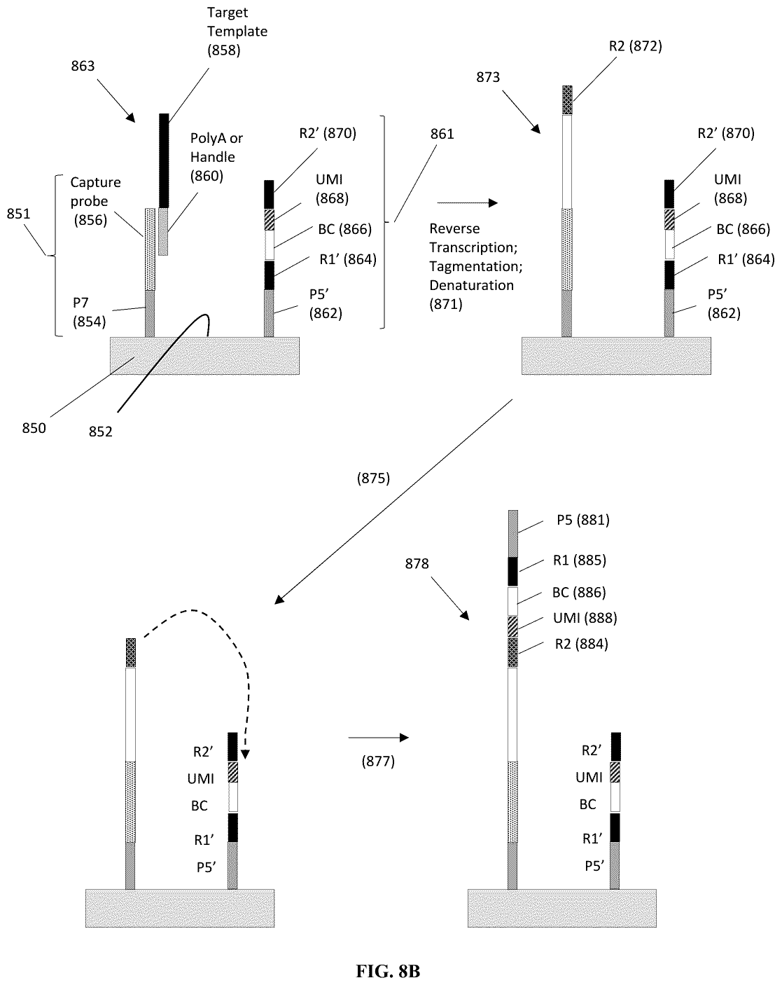

Described here are methods for synthesizing cDNA by utilizing template switching oligonucleotides. FIG. 8A illustrates an example of using template switching primers. Instead of using free-in-solution template switch on the oligonucleotides, surface primers can be used as template switching primers. This improvement can increase the efficiency of the template switching process and also avoid formation of concatemers during the template switching process thus increasing the efficiency of mRNA capture for the entire workflow. In some embodiments, the method comprises preparing a set of cDNA molecules or derivatives thereof from one or more nucleic acid molecules. In some embodiments, the method comprises providing a solid support comprising one or more nucleic acid molecule capture probes and a plurality of surface primer probes, where at least a subset of the plurality of surface primer probes comprise a template switch moiety (e.g., a template switch primer). In some embodiments, the method comprises contacting the solid support with the one or more nucleic acid molecules to yield one or more captured nucleic acid molecule and synthesizing a cDNA molecule from the captured nucleic acid molecule or a derivative. In some embodiments, an adapter can be inserted at the 3′ end of the cDNA molecule or a derivative thereof. In some embodiments, the cDNA molecule or a derivative can be amplified for sequencing.

In some embodiments, the method comprises synthesizing comprises performing one or more second strand synthesis reactions comprising the cDNA molecule or a derivative thereof. In some embodiments, the one or more second strand synthesis reactions are mediated by the subset of the plurality of surface primer probes comprising the template switch moiety. In some embodiments, the second strand is synthesized by template switch extension. In some embodiments the cDNA molecule or the derivative thereof is coupled to the plurality of surface primer probes or the template switching moiety. In some embodiments, the adapter inserted at the 3′ region of the cDNA comprises a sequence configured to permit initiation of a sequencing reaction on a cDNA molecule or derivative thereof. In some embodiments, the one or more nucleic acid molecule capture probes or the surface primer probes can be inactivated or blocked by any one of the moiety described herein.

In some embodiments, the synthesis or amplification of the cDNA occurs when the one or more nucleic acid molecules or the cDNA molecules are attached to the solid support. In some embodiments, the synthesis or amplification of the cDNA occurs when the one or more nucleic acid molecules or the cDNA molecules are eluted off the solid support. In some embodiments, the synthesis or amplification of the cDNA occurs when the one or more nucleic acid molecules or the cDNA molecules are contacted with in-solution primer sequences described herein. In some embodiments, the synthesis or amplification of the cDNA occurs when the one or more nucleic acid molecules or the cDNA molecules are contacted or encapsulated with gel matrix described herein. In some embodiments, the synthesis or amplification of the cDNA occurs when the one or more nucleic acid molecules or the cDNA molecules are eluted off the solid support and contacted or encapsulated with gel matrix.

In some embodiments, the method described herein can be utilized to for obtaining transcriptome sequencing. In some embodiments, the first strand synthesis can be expanded using primer probes comprising randomers. The randomers can include the same combinations of sample barcodes and unique molecular identifiers in addition to other adapter and primer binding sites, such as a transposome adapter sequence or template switching (TS) primer. Template switching and second strand synthesis can be performed for oligo-dT primed cDNA synthesis. The resulting double stranded cDNAs can then be subjected to tagmentation.

FIG. 8B illustrates an embodiment in which capture sequences and barcode sequences (which may or may not include UMIs) are grouped with, or located on, separate surface primers. This results in shorter surface primers, which is advantageous both from a synthesis perspective and also because it creates fewer problems with surface tagmentation (if used). Moreover, the system is modular, so multiple different capture probes can be used without having to alter the design of the rest of the system. For instance a polyDT oligo for mRNA capture, and a handle sequence to capture specific nucleic acid sequences such as complementary oligo barcodes

In an embodiment, solid support (850) has surface (852) comprising (i) capture element or probe (851) which comprises surface primer (854) (which may be, for example, a P7 primer) and capture probe (856) and (ii) oligonucleotide sequence (861) which comprises surface primer (862) (which may be, for example, a P5 surface primer), complement (R1′)(864) of primer binding site (R1) (which may be used in a sequencing step, e.g. to identify the barcode or UMI), barcode sequence (866), UMI (868) and complement (R2′)(870) of primer binding site, R2 (which may be used in a sequencing step, e.g. to identify a target template). Also shown is captured sequence (863) which comprises complement (860) to capture probe (856) (which may be a polyA region of mRNA, for example, or a so-called “handle” sequence of another cellular nucleic acid molecule or an artificial sequence, such as, an antibody label) and a template region (858) which is copied to form part of a cDNA. After reverse transcription, tagmentation (or like procedure) and denaturation (871), preliminary cDNA (873) is produced. In alternative embodiments, R2 (872) may be attached via template switching if tagmentation is not used. Under hydridization conditions and in the presence of a polymerase and dNTPs, R2 (872) anneals to its complement R2′ (870) and is extended (875) copying surface primer (861). This results (877) in final cDNA product (878) comprising primer binding site R2 (884), UMI (888), barcode (886), primer binding site R1 (885), and primer binding site P5 (881). This final cDNA product may, for example, be surface amplified to form clusters for sequencing, or it may be copied and copies eluted for external sequencing.

In some embodiments, the above method for synthesizing barcoded cDNAs may be implemented by the following steps: (a) providing a solid support, wherein the solid support comprises one or more nucleic acid molecule capture probes and a plurality of surface primer probes, wherein at least one of the surface primer probes comprises a barcode sequence; (b) contacting the solid support with said one or more nucleic acid molecules to yield one or more captured nucleic acid molecules; (c) synthesizing an initial cDNA molecule from the captured nucleic acid molecule or a derivative wherein the cDNA molecule is coupled to said solid support; (d) ligating an adaptor to a free end of the cDNA molecule wherein the adaptor comprises a 3′ segment complementary to a 3′ end of a surface primer probe that comprises a barcode sequence; providing conditions wherein the 3′ segment of the adaptor anneals to the 3′ end of a surface primer and is extended, thereby producing a second cDNA molecule comprising a barcode sequence. In some embodiments, the adaptor is ligated to the initial cDNA by tagmentation. In some embodiments, the second cDNA molecule is surface amplified to form a cluster.

### Amplifying Nucleic Acid Molecules Prior To Tagging

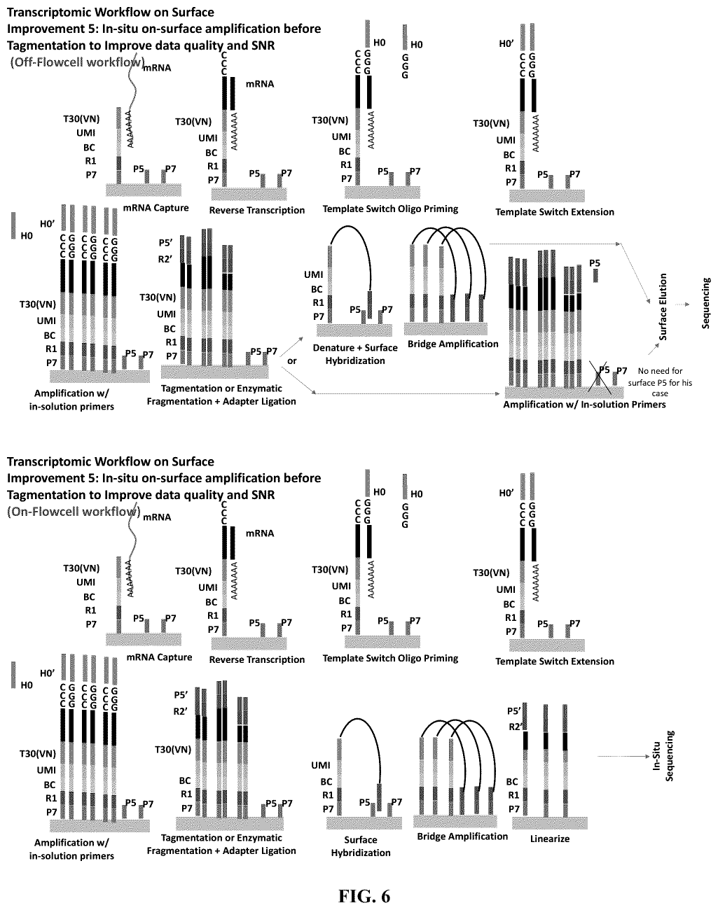

Described herein are methods for amplifying nucleic acid molecules before tagging or fragmentation. FIG. 6 illustrates a non-limiting example of amplifying the cDNA before fragmentation to improve a workflow for obtaining and increasing the quality of singe cell transcriptome in an off-flow cell workflow (top panel) and on-flow cell workflow (bottom panel). In some embodiments, the method comprises preparing a set of cDNA molecules or derivatives thereof from one or more nucleic acid molecules by providing a solid support, where the solid support comprises one or more nucleic acid molecule capture probes. In some aspects, the method comprises contacting the solid support with the one or more nucleic acid molecules to yield one or more captured nucleic acid molecules. In some cases, the method comprises synthesizing a cDNA molecule from the captured nucleic acid molecule or a derivative and amplifying the cDNA molecule or a derivative thereof to generate an amplified cDNA population. As stated previously, an adapter can be inserted at the 3′ region of the amplified cDNA molecule or a derivative thereof, for generating a tagged amplified cDNA population. In some embodiments, the method comprises performing solid-supported amplification on the tagged amplified cDNA population to generate the set of cDNA molecules or derivatives thereof. In some embodiments, the method comprises performing amplification after the cDNA molecules or derivatives thereof are eluted off the solid support. In some embodiments, the cDNA molecule or the derivative thereof, the amplified cDNA population, the tagged amplified cDNA population, the set of cDNA molecules or derivates thereof, or any combination thereof is coupled to the plurality of surface primer probes.

In some embodiments, the adapter comprises a sequence configured to permit initiation of a sequencing reaction on a cDNA molecule of the set of cDNA molecules or derivatives thereof. In some embodiments, the method comprises contacting the solid support with a moiety configured to inactivate at least a subset of the one or more nucleic acid molecule capture probes. In some embodiments, the subset of the one or more nucleic acid molecule capture probes comprise one or more nucleic acid molecule capture probes that did not capture a nucleic acid molecule. In some embodiments, one or more of the nucleic acid molecule capture probes or the surface primer probes can be inactivated or blocked by any one of the moieties described herein.

### Embodiments Employing Polymer Matrices

The present disclosure provides systems for compartmentalizing or isolating one or more biological components. The system can include a fluidic device containing or including one or more biological components. The fluidic device may contain or include one or more polymer precursors. In some cases, the fluidic device can comprise a first surface configured to couple or receive at least one of the one or more biological components to form a coupled biological component. The systems may also include at least one energy source, wherein the energy source is in communication with the fluidic device. In various embodiments, the at least one energy source may form a polymer matrix on or adjacent to at least a portion of the one or more biological components.

In some cases, a sample may be introduced of provided to the system. In certain cases, the sample may comprise one or more biological components. The system may be used to separate one or more biological components from one another. In various cases, the biological components may be physically separated. In some cases, the biological components may be in fluidic communication with one another. In certain cases, the biological components may be in chemical communication with one another. The system may be used for single-cell analysis. In some embodiments, the system may be used for single-cell analysis on a genome level. For example, the system may be used for genome sequencing. For another example, the system may be used for deoxyribonucleic acid (DNA) sequencing. The system may be used for DNA sequencing of cell-free DNA, whole genome sequencing, whole exome sequencing, targeted sequencing, or 16S sequencing. The system may be used for studying DNA tags attached to biomolecules of interest. The biomolecules may comprise proteins, metabolites, etc. In some cases, the DNA may be a nuclear DNA or a mitochondrial DNA. The system may be used for single-cell or bulk analysis on a transcriptome level. For example, the system may be used for ribonucleic acid (RNA) sequencing. For example, the system may be used for 3′ or 5′ gene expression analysis, immune repertoire study of a cell, or full-length mRNA analysis. In some embodiments, the system may be used for single-cell analysis on a proteome level. The system may be used for functional assay(s) of a biological component. The system may be used for studying surface proteins, secreted proteins, or metabolites of a biological component. In some cases, the system may be used to study epigenomics, DNA methylation, or chromatin accessibility in a biological component. The system may be used for other suitable assays, experiments, and processes.

In certain embodiments, the system may be used for single-cell analysis on an indirect cell-cell interaction level. For example, an effect of one or more molecules produced from a first cell on a second cell can be analyzed using the system as provided herein. In various embodiments, the system may be used for analyzing direct cell-cell interactions. For example, two or more cells (e.g., a first cell and a second cell) can be in physical contact and the effect or effects of the first cell on the second cell, or vice versa, can be analyzed using the system as disclosed herein. In some embodiments, the system may be used for drug response analysis in a biological component. In certain embodiments, the system may be used for analyzing a biological component's response to various physiological conditions (e.g., various media, temperature, mechanical stimuli, etc.).

In some cases, the sample comprises a biological sample. The biological sample may comprise a biological component. In some embodiments, the biological sample may comprise 1, 2, 3, 4, 5, 6, 7, 8, 9, 10, 15, 20, 30, 40, 50, 100, 200, 300, 400, 500, 600, 700, 1,000, 10,000, 10⁵, 10⁶, 10⁷, 10⁸, 10⁹, 10¹⁰, 10²⁰, or more biological components. The biological sample may include any number of biological components between any of the two numbers mentioned herein. In some embodiments, the biological sample may comprise more than 10²⁰ biological components. The biological component may comprise a cell. In some embodiments, the cell may comprise a eukaryotic cell, a prokaryotic cell, a fungal cell, a protozoan, an algal cell, a plant cell, an animal cell (e.g., a human cell), or any other suitable cell. The biological component may comprise a cell, a virus, a bacterium, a nucleic acid (e.g., DNA, or RNA), a protein, or a combination thereof. The combination may comprise a DNA-protein complex, an RNA-protein complex, or a combination thereof. In certain embodiments, a nucleic acid may comprise DNA. The DNA may be at least 10 base pair (bp) long. In some embodiments, the DNA is at least 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp long, or longer than 800 bp.

In certain embodiments, one or more polymer precursors may be added to or included with the biological sample. One or more biological samples and one or more polymer precursors may be introduced into the system (e.g., into the fluidic device of the system). The one or more biological samples and the one or more polymer precursors may be introduced into the fluidic device in any order (e.g., in parallel, sequentially, etc.). For example, the biological sample(s) may be introduced prior to the polymer precursor(s), the polymer precursor(s) may be introduced prior to the biological sample(s), the biological sample(s) and polymer precursor(s) may be introduced simultaneously (or substantially simultaneously), or in any other suitable manner or order. In some embodiments, a polymer precursor may include one or more hydrogel precursors. The one or more polymer precursors may be stored and/or introduced separately into the system. In some cases, the one or more polymer precursors may be mixed with the one or more biological components prior to introduction into the system. In various cases, the one or more polymer precursors may be mixed with the one or more biological components after introduction into the system.

The system may comprise a fluidic device. In some embodiments, the fluidic device may include one or more polymer precursors. In other words, one or more polymer precursors may be disposed within at least a portion of the fluidic device (e.g., within at least a portion of a channel of the fluidic device). In some embodiments, the fluidic device may comprise one or more channels or chambers. In some embodiments, the fluidic device may include at least 1, 2, 3, 4, 5, 6, 7, 8, 9, 10, 15, 20, 30, 40, 50, 100, 200, 300, 400, 500, 600, 700, 1,000, 10,000 channels or chambers, or any number of channels or chambers between any of the two numbers mentioned herein. In some embodiments, the fluidic device comprises more than 10,000 channels or chambers. As described herein, the fluidic device may include one or more channels. The fluidic device may also, or alternatively, include one or more chambers. The terms channel and chamber may be used interchangeably in the disclosure herein unless indicated otherwise. For example, a channel or a chamber of the fluidic device may comprise a first surface, a second surface, or more surfaces.

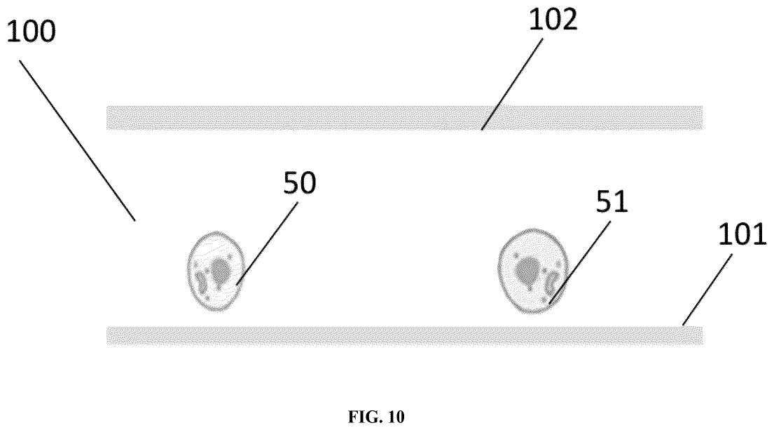

A channel or chamber of a fluidic device may receive or be configured to receive a biological sample. FIG. 10 illustrates a schematic illustration of a portion of a channel 100 that may be disposed in at least a portion of a fluidic device of a system as provided herein. The fluidic device may comprise a channel 100. The channel 100 may comprise a first surface 101. Further, the channel 100 may comprise a second surface 102. In some embodiments, the first surface 101 and the second surface 102 are disposed, placed, or positioned opposite of one another (e.g., as depicted in FIG. 10). In some embodiments, the first surface 101 may be a lower surface. In certain embodiments, the second surface 102 may be an upper surface. The terms “lower” and “upper” are not intended to be limiting and are used herein for convenience when referring to the figures. The channel 100 may receive a biological sample comprising one or more biological components 50, 51. The channel 100 may receive one or more polymer precursors. As illustrated in FIG. 10, the biological components 50, 51 may include cells. However, as discussed herein, the biological components may include tissues, proteins, nucleic acids, etc. In some embodiments, the first surface 101, the second surface 102, or both surfaces may couple or receive, or be configured to couple or receive, at least one of the one or more biological components 50, 51. In some cases, the first surface 101 may couple or receive, or be configured to couple or receive, a biological component (e.g., biological components 50, 51). In certain cases, the second surface 102 may couple or receive, or be configured to couple or receive, a biological component (e.g., biological components 50, 51).

In certain cases, a channel may have a rectangular, circular, semi-circular, oval cross-section, or other suitably shaped cross-section. Accordingly, the channel may have a single, internal surface. In some cases, a channel may have a triangular, square, rectangular, polygonal, or other cross-section. Accordingly, the channel may have three or more internal surfaces. One or more of the internal surfaces may be couple or receive, or be configured to couple or receive, the one or more biological components.

In some cases, the first surface 101, the second surface 102, or both surfaces 101 and 102 may be functionalized, for example, with a coating (e.g., a surface coating). In some embodiments, the surface coating may be a surface polymer. Some non-limiting examples of surface coatings may include a capture reagent (e.g., pyridinecarboxaldehyde (PCA)), a functional group to capture one or more moieties (e.g., a chemical moiety), an acrylamide, an agarose, a biotin, a streptavidin, a strep-tag II, a linker, a functional group comprising an aldehyde, a phosphate, a silicate, an ester, an acid, an amide, an alkyne, an azide, an aldehyde dithiolane, or a combination thereof. In various embodiments, the surface coating may include a functional group to capture one or more moieties. For example, the acrylamide, the agarose, etc. may include such a functional group. In certain embodiments, the surface polymer may comprise polyethylene glycol (PEG), a thiol, an alkene, an alkyne, an azide, or combinations thereof. In various embodiments, the surface polymer may comprise a silane polymer. In some embodiments, the surface polymer may be functionalized with at least one of an oligonucleotide, an antibody, a cytokine, a chemokine, a protein, an antibody derivative, an antibody fragment, a carbohydrate, a toxin, or an aptamer.

In some cases, the first surface 101, the second surface 102, or both surfaces 101 and 102 may comprise one or more barcodes (e.g., nucleic acid barcodes). In some embodiments, the first surface 101, the second surface 102, or both surfaces 101 and 102 may comprise 1, 2, 3, 4, 5, 6, 7, 8, 9, 10, 15, 20, 30, 40, 50, 100, 200, 300, 400, 500, 600, 700, 1,000, 10,000, 50,000, 100,000, 250,000, 500,000, 1,000,000, 2,000,000, 5,000,000, 10,000,000, 15,000,000 barcodes, or any number of barcodes between any of the two numbers mentioned herein. The barcodes may cover an area of about 500 nm² to about 500 nm². In some embodiments, the first surface 101, the second surface 102, or both surfaces 101 and 102 may comprise at most about 10,000,000 total number of barcodes. The barcodes may be different from one another (e.g., each barcode may be unique). In certain embodiments, a first portion or subset of the barcodes may be different from a second portion or subset of the barcodes. There may be 2, 3, 4, 5, 10, 15, 20, 25, 50, 75, 100, 1,000, 10,000 portions or subsets of the barcodes, or any number of portions or subsets of the barcodes between any of the two numbers mentioned herein. In some cases, a barcode (or a portion/subset of barcodes) may be associated with the location of the barcode on a surface (location coordinates (e.g., x-, y-coordinates) on a surface of a channel). A barcode may be attached to or coupled to the captured biological component. In some embodiments, the barcode may be a unique identifier that distinguishes a biological component from other biological components (e.g., that identifies a first biological component versus a second biological component). In some embodiments, a barcode may comprise a nucleic acid sequence (e.g., common sequence) to capture a biological component, or used in amplification. In some embodiments, a barcode may comprise a unique identifier comprising a unique nucleic acid sequence (e.g., DNA sequence, RNA sequence, etc.), protein tag, antibody, or an aptamer. In some embodiments the barcode may comprise a fluorescent molecule. In some embodiments, a location of the captured biological component may be associated with the unique identifier to, for example, retain spatial information of a biological component.

In some embodiments, the fluidic device may be a flow cell. For example, the fluidic device may be used for sequencing (e.g., DNA or RNA sequencing). In some embodiments, the fluidic device may be a microfluidic device. In certain embodiments, the fluidic device may be a nanofluidic device.

The system disclosed herein may comprise one or more energy sources. The energy source may be in communication with the fluidic device. In some cases, the energy source can be used to form one or more polymer matrices in the fluidic device (e.g., on or adjacent to a surface of a channel or chamber of the fluidic device). In some embodiments, the energy source may comprise a light generating device, a heat generating device, an electrochemical reaction generating device, an electrode, or a microwave device. A polymer matrix may be formed in a channel of the fluidic device. The energy source may direct or transfer energy to a predetermined position in the fluidic device. The energy may cause or activate the one or more polymer precursors to form a polymer matrix (e.g., to polymerize) in the predetermined position.

In some embodiments, the polymer matrix may comprise a hydrogel. In some embodiments, the hydrogel may be porous enough, or have pores of a suitable size, to allow movement or transfer of a reagent (e.g., an enzyme, a chemical compound, a small molecule, an antibody, etc.) through the polymer matrix, while the hydrogel may not allow movement or transfer of the biological component (e.g., DNA, RNA, a protein, a cell, etc.) through the polymer matrix. In some embodiments, the pores may have a diameter from 5 nm to 100 nm. In some embodiments, the pores may have a diameter from 5 nm to 10 nm, 10 nm to 20 nm, 20 nm to 30 nm, 30 nm to 40 nm, 50 nm to 60 nm, 60 nm to 70 nm, 70 nm to 80 nm, 80 nm to 90 nm, 90 nm to 100 nm. In some embodiments, the pores may have a diameter larger than 100 nm. In some embodiments, the pores may have a diameter smaller than 5 nm. The reagent may comprise an enzyme or a primer having a size of less than 50 base pairs (bp). A primer may comprise a single-stranded DNA (ssDNA). In some embodiments, a primer may have a size from 5 bp to 50 bp. In some embodiments, a primer may have a size from 5 bp to 10 bp, 10 bp to 20 bp, from 20 bp to 30 bp, 30 bp to 40 bp, or 40 bp to 50 bp. In some embodiments, a primer may have a size of more than 50 bp. In certain cases, a primer may have a size of less than 5 bp. A reagent may comprise a lysozyme, a proteinase K, hexamers (e.g., random hexamers), a polymerase, a transposase, a ligase, a catalyzing enzyme, a deoxyribonuclease, a deoxyribonuclease inhibitor, a ribonuclease, a ribonuclease inhibitor, DNA oligos, deoxynucleotide triphosphates, buffers, detergents, salts, divalent cations, or any other suitable reagent.

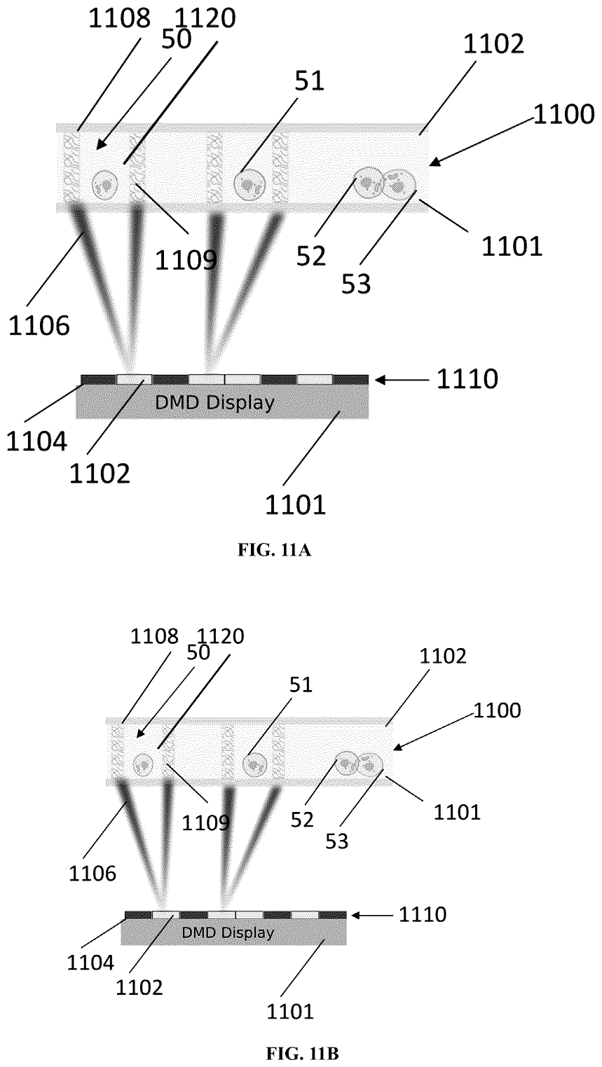

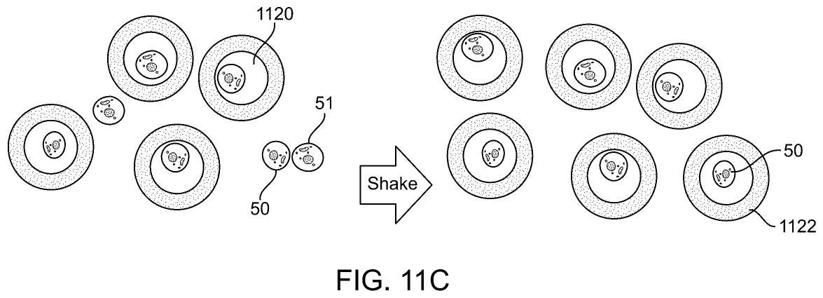

FIG. 11A illustrates a portion of a system as provided herein including an energy source 1103. The embodiment of FIG. 11A may include components that resemble components of FIG. 10 in some respects. For example, the embodiment of FIG. 11A includes a channel 1100 that may resemble the channel 100 of FIG. 10. It will be appreciated that the illustrated embodiments may have analogous features. Accordingly, like features are designated with like reference numerals, with the leading digits incremented to “2.” Relevant disclosure set forth above regarding similarly identified features thus may not be repeated hereafter. Moreover, specific features of the system provided herein, and related components shown in FIG. 11A may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the system and related components of FIG. 11A. Any suitable combination of the features, and variations of the same, described with respect to the system and components illustrated in FIG. 10, can be employed with the system and components of FIG. 11A, and vice versa. This pattern of disclosure applies equally to further embodiments depicted in subsequent figures and described hereafter.

With continued reference to FIG. 11A, the channel 1100 of the system may include a first surface 1101 and a second surface 1102. In some embodiments, the energy source 1103 may comprise one or more energy emitting portions (e.g., an energy emitting portion 1105). In some embodiments, the energy source 1103 may comprise one or more non-emitting portions (e.g., a non-emitting portion 1104). The non-emitting portion 1104 may not emit, or be configured to emit, energy. In some embodiments, the emitting portion 1105 can emit energy in the form of electromagnetic waves (e.g., microwaves, light, heat, etc.) to at least a portion of the fluidic device. In certain embodiments, the emitting portion 1105 can emit energy to the fluidic device. In some embodiments, the fluidic channel may be coupled to on a movable stage. In other embodiments, light may be projected to or onto at least a portion of the fluidic channel to generate one or more polymer matrices. The light may be directed to various parts of the fluidic channel. In some embodiments, the emitting portion 1105 may be coupled to an objective (e.g., a microscope objective or lens), where the objective may be moved to different portions of the fluidic device. The objective may provide a shape (e.g., virtual or physical mask) to allow light to form a pattern on the fluidic device, in order to form a polymer matrix similar or complementary to the pattern. In various embodiments, the one or more polymer precursors in the fluidic device or mixed with the biological sample can absorb emitted energy 1106. In some embodiments, the emitted energy 1106 can form, or be sufficient to form, a polymer matrix from the one or more polymer precursors. For example, a portion of the one or more polymer precursors within the channel 1100 of the fluidic device may be activated by the emitted energy and a polymerization reaction may be initiated to form a polymer matrix.

In some embodiments, the energy source may emit energy to a larger portion of the fluidic channel or almost the entire surface of the fluidic channel. A physical mask may be used to block the energy emitted to one or more portions of the fluidic channel. The energy source (e.g., light source) may be coupled to the fluidic device via an objective (e.g., a microscope objective or lens). The energy source may be directed to a portion of the fluidic channel (e.g., via a movable objective). In some cases, the light source, the objective, and/or the fluidic channel are movable to allow emission of energy to the fluidic channel so as to generate a pattern on at least a portion of a surface of the fluidic device. The polymer matrix may be formed similarly or complementary to the pattern of energy emission.

A polymer precursor may comprise an activating molecule that can absorb the emitted energy 1106 to initiate polymerization of the one or more polymer precursors in the fluidic device. Non-limiting examples of the activating molecule may include a photocatalyst, a photoactivator, a photoacid generator, or a photobase generator. In some embodiments, a first polymer matrix 1108 and/or a second polymer matrix 1109 can be formed on or adjacent to a biological component 50. In certain embodiments, the first polymer matrix 1108 and the second polymer matrix 1109 can form an analysis chamber or compartment 1120 that separates (e.g., physically separates) the biological component 50 from other biological components (e.g., biological components 51, 52, or 53) in the fluidic device. Stated another way, the polymer matrix may compartmentalize the channel (e.g., channel 1100). In various embodiments, the polymer matrix may partially surround a biological component. For example, a polymer structure surrounding a biological component may form a closed structure (e.g., a hollow cylinder-shaped polymeric structure) or a partially open structure (e.g., a crescent-shaped polymeric structure). In some embodiments, two or more polymer matrices may be formed adjacent to a biological component forming a compartment separating the biological component from other biological components. In certain embodiments, the polymer matrix may comprise or form a wall (e.g., a polymer matrix wall).

With continued reference to FIG. 11A, the polymer matrix 1108, 1109, or at least a portion of the polymer matrix 1108, 1109, may be coupled to the first surface 1101, the second surface 1102, or both surfaces 1101 and 1102. In certain embodiments, the polymer matrix, or at least a portion of the polymer matrix, may be coupled to a third surface, a fourth surface, a fifth surface, etc. as appropriate. In various embodiments, the polymer matrix 1108, 1109 may extend from the first surface 1101 to the second surface 1102 (e.g., through at least a portion of a lumen of the channel 200 or a cavity of a chamber) such that the polymer matrix surrounds, or substantially surrounds, the biological component 50. In some embodiments, two or more biological components (e.g., biological components 50, 51 of FIG. 11C) that are in close physical proximity may be separated (e.g., by agitating or shaking the fluidic device). The fluidic device may be agitated or shaken by physical movement, use of a sonic pulse, changing a flow in the channel, or any other suitable method of agitation. A polymer matrix may then be formed that surrounds (or partially surrounds) the biological components that are separated. FIG. 11B illustrates polymer matrices 1108, 1109 formed surrounding the biological component 50 after being separated from the biological component 51. FIG. 11C illustrates a process, according to various embodiments, of separating the two biological components 50, 51, which are in close proximity. That is, by agitating or shaking the fluidic device the biological components 50, 51 can be separated. In some embodiments, separation of the biological components is achieved through fluidic pressure, flow pulsation, dielectrophoresis, optothermal flow, or some combination thereof. In some cases, separation of the biological components is achieved through acoustic vibration. FIG. 11C also illustrates a polymer matrix being formed to generate a compartment 1122 surrounding the biological component 50 after the separation of the biological components 50, 51.

With continued reference to FIG. 11A, in some cases, the energy source 1103 can, or be configured to, form or produce one or more emitting portions 1105 and one or more non-emitting portions 204. The systems disclosed herein may further include a spatial energy modulating element to direct energy from the energy source to one or more targeted portions of the fluidic device. For example, the spatial energy modulating element may be configured to selectively direct the energy from the energy source to form a polymer matrix on at least a portion of or adjacent to a biological component. The spatial energy modulating element may be configured to selectively direct the energy by, for example, inhibiting or preventing energy from being directed to one or more portions other than the one or more targeted portions of the fluidic device. In some embodiments, the spatial energy modulating element may comprise a physical mask. In some cases, the spatial energy modulating element may comprise a virtual mask. In certain cases, the spatial energy modulating element may be configured to control one or more electrodes that can selectively provide energy to the one or more targeted portions of the fluidic device. The electrode concept may also be used to provide spatially modulated energy to form the hydrogel structure. In some implementations, one or more electrodes can be arranged at predetermined locations in the fluidic channel, thus allowing formation of the hydrogel in those locations. In alternative implementations, the electrodes can be in the form of an array. The elements of the array can be turned on or off on demand to create the desired spatial pattern of energy to form the desired shape of the hydrogels. For example, one or more electrodes (e.g., an array of electrodes) may be disposed within one or more portions of the fluidic device. For another example, one or more electrodes (e.g., an array of electrodes) may be in communication (e.g., electrical communication) with one or more portions of the fluidic device.

In some embodiments, a mask may prevent, or be configured to prevent, one or more portions of the energy emitting surface 1110 of the energy source 1103 from emitting energy (e.g., non-emitting portions 1104). In some embodiments, the mask may be a virtual mask (e.g., a computer code or a digital system). In certain embodiments, the mask can prevent the energy from being emitted to a location where a biological component is present. This may allow or permit forming a polymer matrix adjacent to, on, or encapsulating the biological component (e.g., to retain a cell, proteins, DNA molecules, RNA molecules, or other target molecules at a location on the fluidic channel). In other embodiments, the mask may facilitate the polymerization such that the polymer matrix is on the biological component. In various embodiments, the mask may be a physical mask (e.g., an opaque material, a thermal shield, or an electromagnetic shield). In some embodiments, the mask (e.g., a virtual mask or a physical mask) can be generated using, or in combination with, a detector that detects or identifies a location of a biological component. In some embodiments, the detector comprises a camera. In some embodiments, the detector comprises a light detector, conductivity detector, an ultrasound detector, an ultrasonic sensor, a piezoelectric sensor, a combination thereof, or another suitable detecting device.

In some embodiments, the first surface 1101 or the second surface 1102 may comprise a detector that detects, or is configured to detect, one or more locations of one or more biological components in the fluidic device (e.g., in the channel 1100). In certain embodiments, the energy source 1103 can comprise, be coupled to, or be in communication with a detector that detects, or is configured to detect, a location of a biological component in the fluidic device. In various embodiments, a mask may be generated using an image obtained from at least a portion of the fluidic device. The mask may allow or permit the energy source 1103 to emitting energy in or toward one or more locations or positions where one or more biological components are present on or adjacent the first surface 1101. The mask may inhibit or prevent the energy source 1103 from emitting energy in or toward one or more locations or positions where one or more biological components are present on or adjacent the first surface 1101. In some embodiments, the image may be obtained from a camera (e.g., a digital camera, fluorescent imaging camera, etc.). In some embodiments, the camera may be coupled to, connected to, or in communication with the energy source 1103. For example, the camera (not shown) may be in electrical communication with the energy source 1103. In some embodiments, the energy source 1103 may comprise the camera. In various embodiments, the energy source 203 may comprise a microscope (e.g., a fluorescence microscope, a confocal microscope, lens-free imaging system, a transmission electron microscopy (TEM), a scanning electron microscope (SEM), etc.). The microscope may be used to detect one or more positions of one or more biological components (e.g., in combination with the detector).

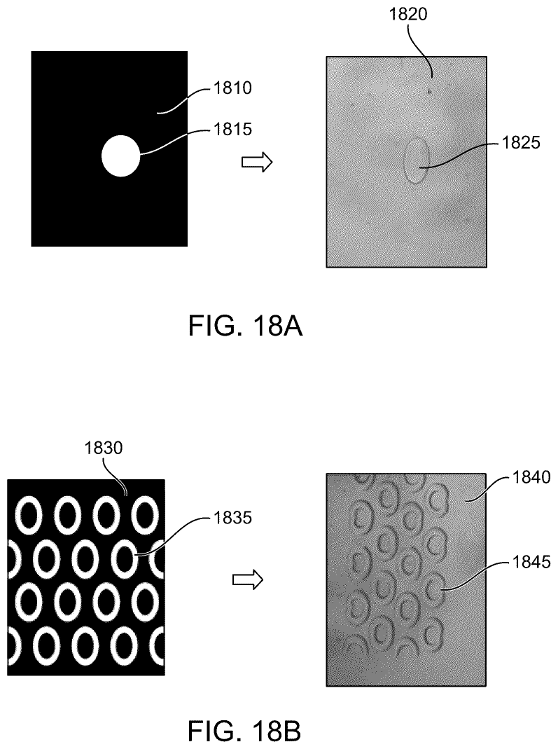

FIG. 18A illustrates an example of a mask comprising an energy masking region 1810 and an energy transparent region 1815. Energy from an energy source may be blocked by the energy masking region 1810 to prevent the energy to form any polymer matrix in a portion of the fluidic device (e.g., portion 1820). Energy transparent region 1815 may allow the energy to communicate with the fluidic device to form a polymer matrix 1825. FIG. 18B illustrates another example of a mask, where the energy transparent region 1835 is in shape of a hollow cylinder (e.g., donut). Energy being masked by a masking region 1830 may prevent energy communication with a portion of the fluidic device (e.g., a portion 1840). The energy transparent region 1835 may deliver energy to the fluidic device to form a polymer matrix 1845. The polymer matrix 1845 may be in shape of a hollow cylinder.

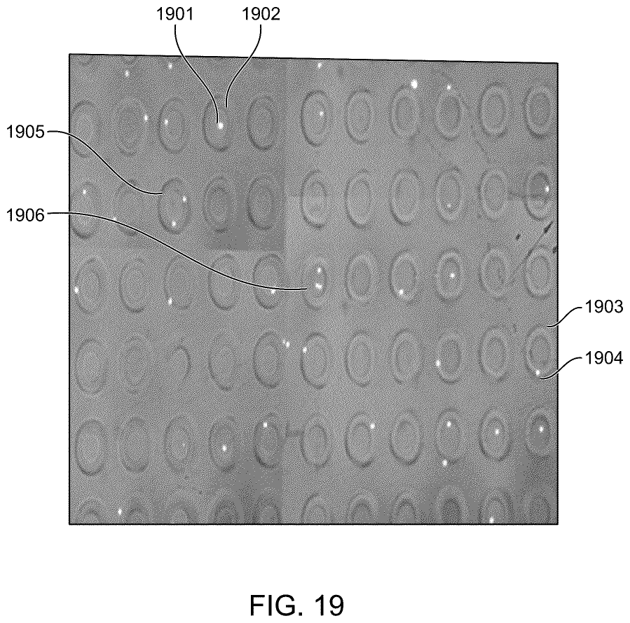

FIG. 19 illustrates an example of biological components (i.e., indicated as white spots) encapsulated and/or localized using polymer matrices. In some cases, a biological component 1901 may be localized within a hollow region of a polymer matrix compartment 1902. In some other cases, a polymer matrix 1903 may be formed on a biological component 1904. In some alternative cases, a polymer matrix 1905 may localize more than one biological component. A biological compartment polymer matrix 1906 may encapsulate one or more biological components.

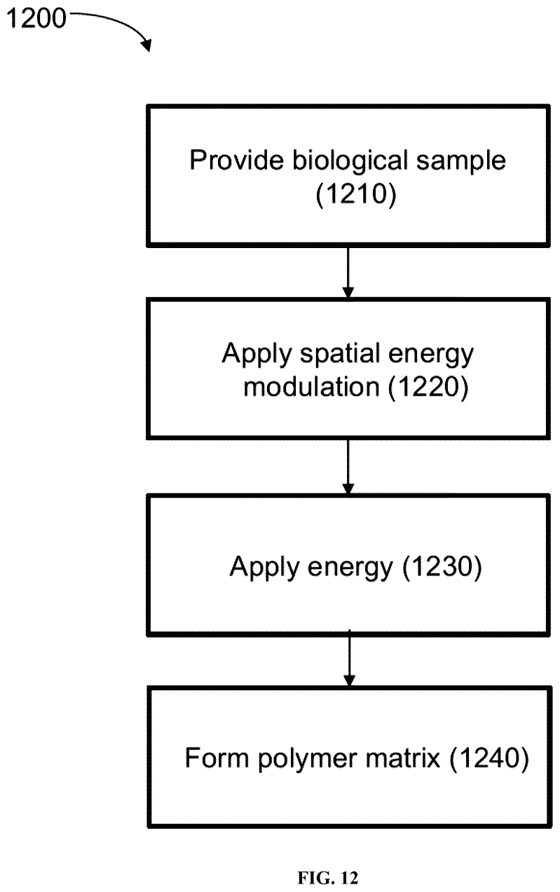

FIG. 12 is a flow chart of forming a polymer matrix on or adjacent to one or more biological components, according to some embodiments of the present disclosure. The process 1200 may be performed manually or automatically (e.g., by an appropriately programmed computer system). In step 1210, a biological sample may be deposited, introduced, or provided into at least a portion of the fluidic device. In some embodiments, a mask may then be formed or generated to render one or more portions of the energy source directed towards a biological component non-emitting (step 1220). In step 1230, the energy source may apply or provide energy to at least a portion of the fluidic device. In some embodiments, the energy source can activate or initiate polymer precursors such that the polymer precursors form a polymer matrix (e.g., via energy provided by the energy source). In some embodiments, an imaging of the fluidic device can be performed subsequent to step 1210 and prior to step 1220 to determine or identify a location of the biological components to generate a mask. In some embodiments, the mask is a virtual mask. In some embodiments, the polymer matrix may form a compartment that partially or completely surrounds a biological component.

In certain cases, the energy source may be manipulated such that the polymer matrix is formed in different steps. For example, the energy source may initiate a plurality of polymer precursors such that the polymer precursors form an open compartment (e.g., a crescent shape or half-cylinder polymer matrix). The open compartment may operate to capture and/or contain a biological component (e.g., a cell), or a portion of a sample, to a portion of the fluidic device. The orientation of the energy source or the fluidic device may be adjusted, and an additional portion of polymer matrix may be formed. This additional portion may be used to form one or more compartments in conjunction with the pre-formed half-cylinder polymer matrix. In other embodiments polymer matrix compartments can be formed in at least 2, 3, 4, 5, or more matrix-forming steps.

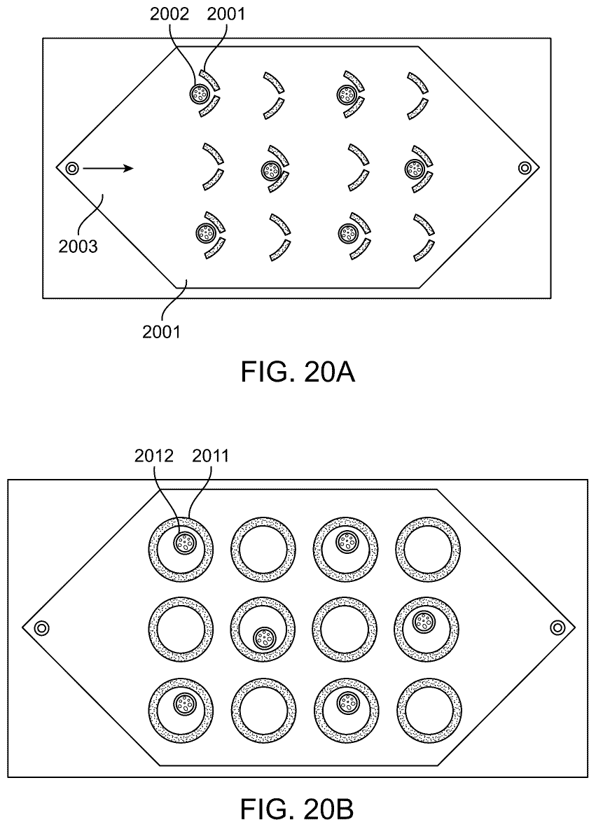

FIG. 20A and FIG. 20B show an example of multi-step polymer matrix compartment generation. FIG. 20A illustrates a first step of the multi-step generation, where open compartments (e.g., an open compartment 2001 made from a polymer matrix) may be generated to capture and/or contain a biological component (e.g., a biological component 2002). A sample comprising the biological component 2002 may have a flow direction 2003 within the fluidic device (e.g., a portion of a fluidic device 2000). The open compartment 2001 may be formed by generating a polymer matrix using an energy source and an energy modulation unit as described herein. The open compartment may intersect a portion of the direction of the flow 2003 of the sample in the fluidic device. The polymer matrix open compartment 2001 may be oblique or perpendicular to the direction of the flow 2003 of the sample in the fluidic device. FIG. 11B illustrates a second step of the multi-step generation, where the open compartments (e.g., open compartment 2001) are sealed off or closed by forming polymer matrix adjacent, around, or on the biological component (e.g., biological component 2012). In some cases, in the second step a biological component may be completely or substantially completely encapsulated by the polymer matrix (e.g., to form a closed compartment 2011). In some cases, the polymer matrix that may form adjacent, around, or on the biological component localizes the biological component to a location on the fluidic device 2000. Genomic and/or proteomic material may be extracted from the localized biological component. The polymer matrix may further localize the extracted materials. The fluidic device may then provide a surface where the extracted material can be sequenced. In some embodiments, the extracted materials may be eluted and transferred to another device or surface for sequencing. In other embodiments, the sequencing may be performed through short-read sequencing, nanopore sequencing, sequencing by synthesis, sequencing by in situ hybridization, any optical readout using a microscope, or any other suitable method of sequencing.

One or more surfaces of the fluidic device may comprise an optical (e.g., fluorescence), mechanical, electrical, or biochemical sensing element or sensor. The sensing element may comprise a fluorescent tag, an enzyme, a primer, an oligonucleotide, or a sensor molecule (e.g., a biochemical sensor molecule). The sensing element may be used to detect and/or measure a pH, an oxygen concentration, a CO₂ concentration, or any other suitable variable. The sensing element may detect and/or measure a parameter locally. For example, the sensing element may detect and/or measure a pH, an oxygen concentration, or a CO₂ concentration within a compartment (e.g., a polymer matrix shell cylinder) surrounding the biological component.

### Systems with Capture Elements

The present disclosure also provides systems including one or more capture elements for immobilizing and/or compartmentalizing one or more biological components (e.g. the nucleic acid capture probes described herein). The system can include a fluidic device. The fluidic device can include or contain one or more biological components. Further, the fluidic device can include or contain one or more polymer precursors. In some embodiments, the fluidic device can include a first surface (e.g., in a channel and/or chamber of the fluidic device). The fluidic device can include one or more capture elements. The capture elements can immobilize, or be configured to immobilize, at least one of the one or more biological components at a location on or adjacent to the first surface (or any suitable surface). Immobilization or coupling of a biological component to a capture element can form an immobilized biological component. The system may further include at least one energy source in communication with the fluidic device. In certain embodiments, the at least one energy source can provide or supply energy, or be configured to provide or supply energy, to at least a portion of the fluidic device. Accordingly, the energy source can activate or cause the one or more polymer precursors (e.g., disposed in the fluidic device) to form at least one polymer matrix on or adjacent to an immobilized biological component. In various embodiments, the fluidic device may further include a platform or a stage to hold the fluidic device. In some embodiments, the system may also include a sequencing device (e.g., a next-generation sequencing device) to obtain sequencing data. The polymer matrix formed in the fluidic device may be used to capture and localize a biological component. Genomic and/or proteomic material may be extracted using the fluidic device. The fluidic device may then provide a surface where the extracted material can be sequenced. In some embodiments, the extracted materials may be eluted and transferred to another device or surface for sequencing. In other embodiments, the sequencing may be performed through short-read sequencing, nanopore sequencing, sequencing by synthesis, sequencing by in situ hybridization, or any optical readout using a microscope.

In order to immobilize a biological component, a fluidic device may comprise one or more capture sites. A capture site may include a capture element. In some embodiments, the one or more capture elements or sites may comprise or be disposed in a pattern. A fluidic device may comprise 1, 2, 3, 4, 5, 6, 7, 8, 9, 10, 15, 20, 30, 40, 50, 100, 200, 300, 400, 500, 600, 700, 1,000, 10,000, 10⁵, 10⁶, 10⁷, 10⁸, 10⁹, 10¹⁰, 10²⁰ capture elements, or any number of capture elements between any of the two numbers mentioned herein. In some embodiments, the fluidic device may comprise more than 10²⁰ capture elements.

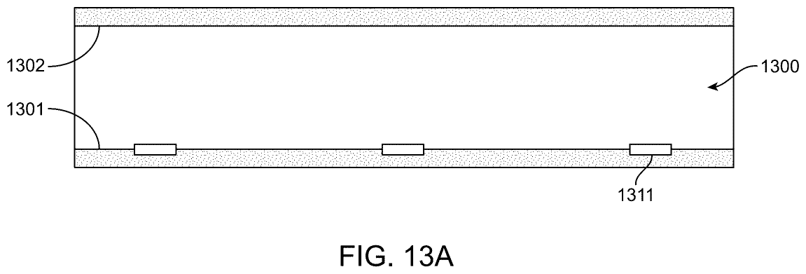

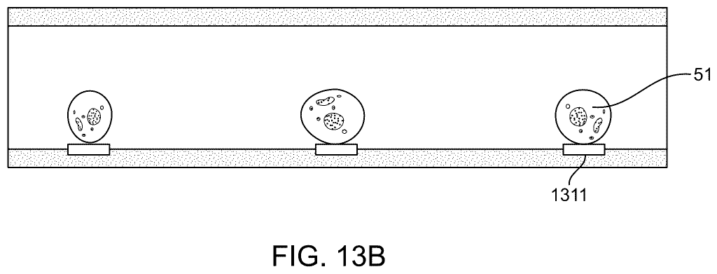

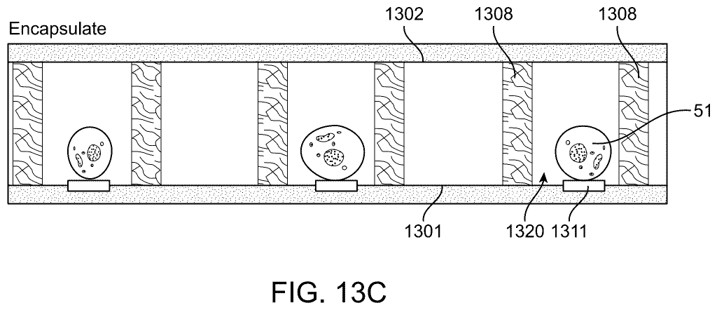

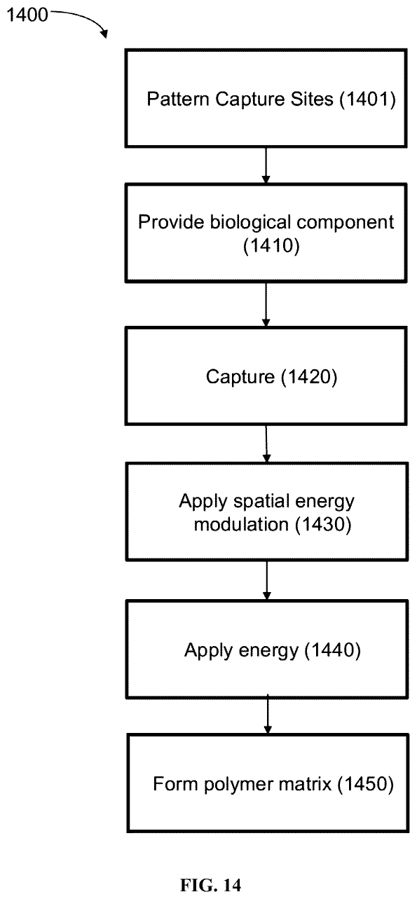

The fluidic device may comprise a channel. The fluidic device may comprise a chamber. FIG. 13A illustrates an example of at least a portion of a channel 1300 in a fluid device. One or more capture elements 1311 may be disposed or positioned on a first surface 1301 of the fluidic device. In some cases, a second surface 1302 may comprise one or more capture elements. The capture elements may be disposed on both surfaces or any other suitable surface. A capture element may comprise or be at least partially formed by a functional group. Some non-limiting examples of functional groups include a capture reagent (e.g., pyridinecarboxaldehyde (PCA)), a biotin, a streptavidin, a strep-tag II, a linker, or a functional group that can react with a molecule (e.g., an aldehyde, a phosphate, a silicate, an ester, an acid, an amide, an alkyne, an azide, or an aldehyde dithiolane). The functional group may couple specifically to an N-terminus or a C-terminus of a peptide. The functional group may couple specifically to an amino acid side chain. The functional group may couple to a side chain of an amino acid (e.g., the acid of a glutamate or aspartate, the thiol of a cysteine, the amine of a lysine, or the amide of a glutamine or asparagine). The functional group may couple specifically to a reactive group on a particular species, such as a membrane-bound molecule on a cell (e.g., a glycoprotein of a eukaryotic cell or a pilus on a plasma of a prokaryote). In some examples, the capture elements can comprise fibronectin. In another example, the capture elements can comprise RGD peptides. In some cases, capture elements may comprise antibodies. In some examples, the functional motif can be reversibly coupled and cleaved (e.g., by using an enzyme). FIG. 13B illustrates an example of a biological component 51 in contact with or coupled to a capture element 1311. In some cases, a repelling surface coating (e.g., PEG) may be used to prevent the polymer matrix form covering or trapping a biological component.

In various instances, the capture element may comprise a physical trap, a hydrodynamic trap, a geometric trap, a well, an electrochemical trap (e.g., trapping charge molecules), streptavidin, an antibody, an aptamer, affinity binding (e.g., a peptide that may bind to a surface protein of a cell), one or more magnetic material (e.g., magnetic disk, magnetic array, or magnetic particles), a dielectrophoretic trap (e.g., electrode array), or a combination thereof. The trap may comprise a polymer matrix or hydrogel. The polymer matrix or hydrogel trap may be constructed or deconstructed on demand using an energy source and/or degradation similar to the polymer matrix compartments mentioned herein. For example, a capture element may comprise a well. The well may be from 1 μm to 50 μm in diameter. In some embodiments, the well may be from 1 μm to 20 μm, 20 μm to 30 μm, 30 μm to 40 μm, or 40 μm to 50 μm in diameter. The well may be more than 50 μm in diameter. The well may be less than 1 μm in diameter. In some embodiments, the well may be from 0.1 μm to 100 μm in depth. In certain embodiments, the well may be more than 100 μm in depth. The well may be less than 0.1 μm in depth. The depth of the well may be from 0.1 μm to 0.5 μm, 0.1 μm to 1 μm, 0.1 μm to 5 μm, 0.1 μm to 10 μm, 0.1 μm to 20 μm, 0.1 μm to 30 μm, 0.1 μm to 50 μm, 0.1 μm to 100 μm, 0.5 μm to 1 μm, 0.5 μm to 5 μm, 0.5 μm to 10 μm, 0.5 μm to 20 μm, 0.5 μm to 30 μm, 0.5 μm to 50 μm, 0.5 μm to 100 μm, 1 μm to 5 μm, 1 μm to 10 μm, 1 μm to 20 μm, 1 μm to 30 μm, 1 μm to 50 μm, 1 μm to 100 μm, 5 μm to 10 μm, 5 μm to 20 μm, 5 μm to 30 μm, 5 μm to 50 μm, 5 μm to 100 μm, 10 μm to 20 μm, 10 μm to 30 μm, 10 μm to 50 μm, 10 μm to 100 μm, 20 μm to 30 μm, 20 μm to 50 μm, 20 μm to 100 μm, 30 μm to 50 μm, 30 μm to 100 μm, or 50 μm to 100 μm. The depth of the well may be about 0.1 μm, about 0.5 μm, about 1 μm, about 5 μm, about 10 μm, about 20 μm, about 30 μm, about 50 μm, or about 100 μm. The depth of the well may be at least 0.1 μm, 0.5 μm, 1 μm, 5 μm, 10 μm, 20 μm, 30 μm, or 50 μm. The depth of the well may be at most 0.5 μm, 1 μm, 5 μm, 10 μm, 20 μm, 30 μm, 50 μm, or 100 μm.

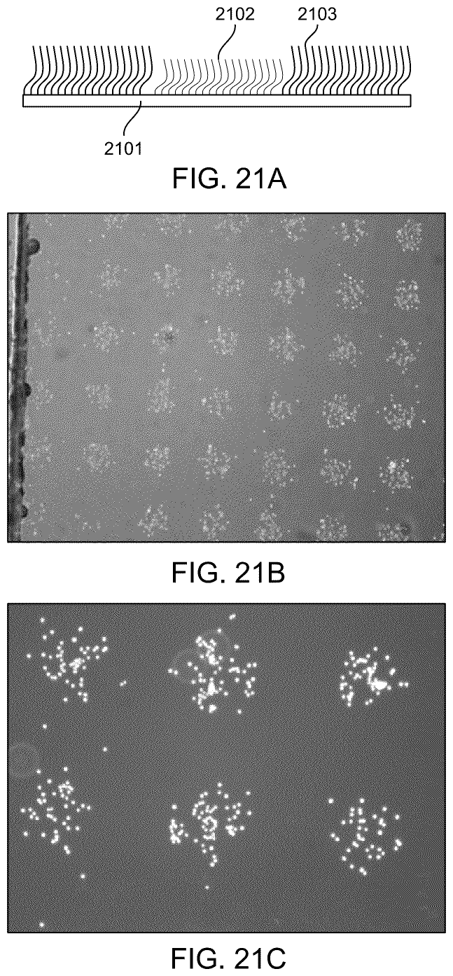

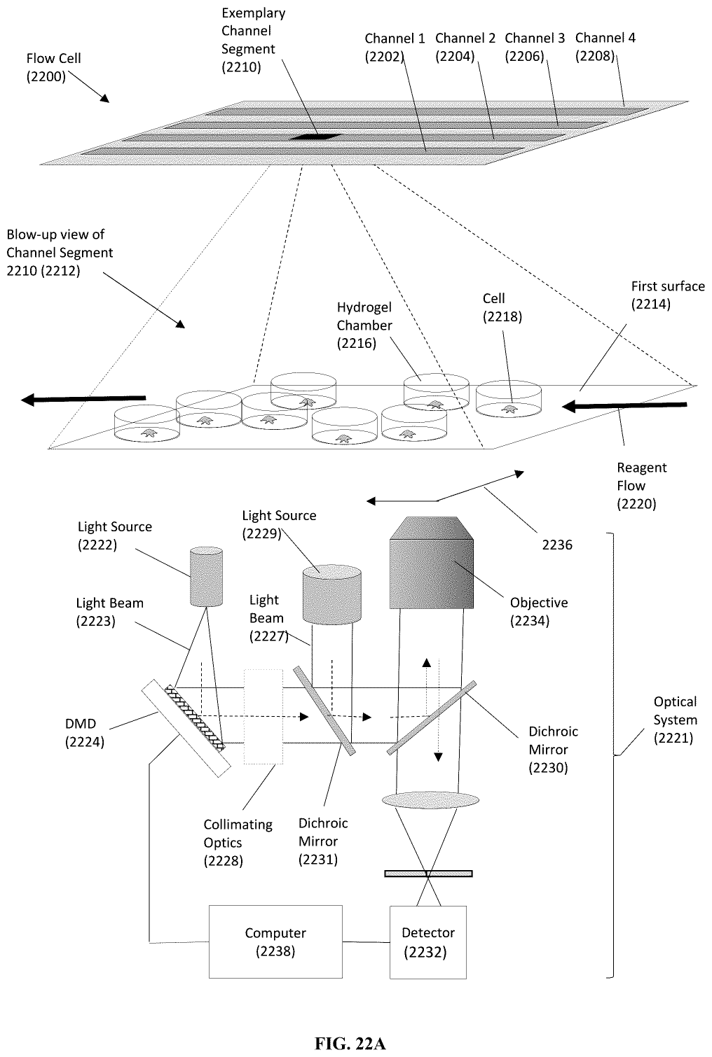

In some embodiments, the fluidic device may comprise a repelling surface coating that may be used to prevent capturing of a biological component at predefined locations. FIG. 21A illustrates a portion of a surface 2101 of a fluidic device, where the surface 2101 may comprise a capturing site 2102 and a repelling site 2103. The surface 2101 may be functionalized using a surface coating (e.g., PEG) to generate the repelling site 2103. The repelling site 2103 may prevent biological components from binding to the surface 2101 at the location of the repelling site 2103 and drive the biological component to the capture site 2102. In some cases, the surface 2101 may only comprise the repelling sites without a capturing site. The repelling site may initially localize the biological components. A polymer matrix may be formed by directing an energy source to the repelling sites to form compartments adjacent to the biological components that may be located in between the repelling sites. FIG. 21B illustrates an example of biological components contained in predefined locations in the fluidic device. FIG. 21C illustrates a higher magnification example of biological components contained in predefined locations in the fluidic device.

An energy source may be used to form a polymer matrix on, around, or adjacent to at least a portion of a captured biological component. In some embodiments, a mask may be used to allow or permit the energy source to direct energy toward a location or position of a captured biological component. In certain embodiments, a mask may be used to inhibit or prevent the energy source from directing energy toward a location or position of a captured biological component. The mask may be configured to direct the energy to predetermined or selected locations to form a polymer matrix surrounding or at least partially surrounding the one or more biological components. The mask may be generated based at least in part on a pattern of the capture sites (e.g., the pattern of the capture sites/elements on a surface of a fluidic device). In some embodiments, the mask may be configured to prevent energy from being directed to a location surrounding a capture site or element which has not captured or coupled a biological component. In certain cases, to analyze a single cell, the mask may be configured to prevent the energy from being emitted adjacent to a location of a capture element which has captured or coupled two or more biological components. In some embodiments, the mask may be configured to allow or permit energy to be emitted adjacent to a location of a capture element which has captured two or more biological components, for example, to allow analysis of cell-cell interactions. In certain embodiments, the mask may be a photolithographic mask or another suitable mask, as described herein. In some embodiments, the system may further comprise a detector, for example, to detect a location of a biological component, as described herein. The mask may be generated based at least in part on the detected location of a biological component. Additionally, the mask may selectively direct or supply energy from the energy source to the fluidic device, as described herein.

FIG. 13C illustrates an example of a method of forming polymer matrices adjacent to (e.g., surrounding) biological components. A polymer matrix 1308 may be formed adjacent to a capture element 1311. The polymer matrix 1308 may be configured to hold a biological component 51 in place or within an analysis chamber or compartment 1320. The compartment 1320 may be formed, at least in part, by the polymer matrix 1308, the first surface 1301, and the second surface 1302 forming a chamber or at least partially sealed-off space within the fluidic device (e.g., around the biological component 51). In some embodiments, the polymer matrix 1308 may form the compartment 1320 surrounding the biological component 51. The compartment 1320 may hold the biological component 51 in place. The polymer matrix 1308 and/or the compartment 420 may inhibit or prevent a compound associated with the biological component 51 from leaving the compartment. In some embodiments, the compound associated with the biological component may comprise a nucleic acid (e.g., DNA or RNA), a protein, a metabolite, an enzyme, an antibody, combinations thereof, or any other suitable compound or material. In some embodiments, the surface of the polymer matrix or hydrogel may be functionalized by coupling a functional group to the polymer matrix or hydrogel. The functionalized surface of the polymer matrix inside the compartment may be coupled to a capturing element (e.g., an antibody) to capture a molecule secreted by the biological component (e.g., secreted protein). The capturing element or the captured molecule may then be read out by a sensing molecule or by a labeling method, for example, by fluorescent labeling. In some embodiments, a polymer matrix may be configured to allow passage of one or more compounds associated with a biological component. In some embodiments, a polymer matrix may be configured to allow passage of a reagent. The reagent may comprise, for example, one or more enzymes, chemicals, oligonucleotides (e.g., one or more primers having a size of less than 50 base pairs), lysozymes, proteinase K, random hexamers, polymerases, transposases, ligases, catalyzing enzymes, deoxynucleotide triphosphates, buffers, cell culture media, divalent cations, combinations thereof, or any other suitable reagent.

In certain embodiments, a first surface, a second surface, or both surfaces of a channel in the fluidic device may be functionalized, as described herein. A surface (e.g., a first surface, a second surface, a third surface, etc.) of the fluidic device may comprise a compound configured to bind to a biological component (e.g., a captured biological component). In some embodiments, a surface (e.g., the first surface, the second surface, the third surface, etc.) of the fluidic device may comprise one or more barcodes. One or more surfaces may comprise oligos to from DNA clusters for sequencing. In some cases, one or more surfaces may comprise one or more nanopore readers for direct DNA and/or RNA readout. One or more surfaces may comprise nanowells to capture single RNA molecules and/or single DNA molecules or to contain a DNA/RNA library. In some alternative cases, one or more surfaces may comprise patterned hydrophobic/hydrophilic features for selective deposition of DNA nanoballs. Nanoballs may be generated by circularization and amplification of DNA libraries from DNA/RNA molecules.

One or more surfaces of the fluidic device may comprise an optical (e.g., fluorescence), mechanical, electrical, or biochemical sensing element or sensor. The sensing element may comprise a fluorescent tag, an enzyme, a primer, an oligonucleotide, or a sensor molecule (e.g., a biochemical sensor molecule). The sensing element may be used to detect and/or measure a pH, an oxygen concentration, a CO₂ concentration, or any other suitable variable. The sensing element may detect and/or measure a parameter locally. For example, the sensing element may detect and/or measure a pH, an oxygen concentration, or a CO₂ concentration within a compartment (e.g., a polymer matrix shell cylinder) surrounding or encapsulating the biological component.

In some embodiments, the fluidic devices described herein comprise a nucleic acid molecule capture probe and a plurality of surface primer probes. In some embodiments, the nucleic acid molecule capture probe is located within a one or more compartments as described herein (e.g. well, polymer matrix). In some embodiments, the nucleic acid molecule capture probe is located adjacent to one or more compartments. In some embodiments, the surface primer probes are located within a one or more compartments as described herein (e.g. well, polymer matrix). In some embodiments, the surface primer probes are located adjacent to one or more compartments. In some embodiments, the nucleic acid molecule capture probe and/or the surface primer probes comprise an adapter sequence. In some embodiments, the nucleic acid molecule capture probe and/or the surface primer probes comprise amplification primer sequences. In some embodiments, the adapter comprises a sequence configured to permit initiation of a sequencing reaction on nucleic acid molecule or derivatives thereof (e.g. cDNA). In some embodiments, the microfluidic device comprises a moiety configured to inactivate at least a subset of one or more nucleic acid molecule capture probes. In some embodiments, the subset of the one or more nucleic acid molecule capture probes comprise one or more nucleic acid molecule capture probes that are not occupied by a nucleic acid molecule. In some embodiments, the moiety configured to inactivate at least the subset of the one or more nucleic acid molecule capture probes comprises an exonuclease. In some embodiments, the nucleic acid molecule capture probe and/or the surface primer probes comprise template switch oligos. In some embodiments, the microfluidic device comprises one or more compartments for the insertion of in-solution primer sequences. In some embodiments, at least a subset of the plurality of surface primer probes comprises a blocking agent that blocks an extension reaction on the at least the subset of the plurality of surface primer probes. In some embodiments, the blocking agents can be removed by a reaction that unblocks the at least the subset of the plurality of surface primed probes to permit the extension reaction. In some embodiments, the one or more blocking agents comprise one or more 3′ phosphate nucleotides. In some embodiments, the one or more blocking agents comprise a nucleic acid molecule comprising a sequence complementary to at least the subset of the plurality of surface primer probes. In some embodiments, the one or more blocking agents comprise a nucleic acid molecule comprising a sequence partially complementary to at least the subset of the plurality of surface primer probes, a reversible terminator nucleotide and a polymerase, or any derivatives thereof. In some embodiments, the microfluidic device comprises reagents sufficient for sequencing the at least the subset of the nucleic acid molecules or derivatives thereof in situ on the microfluidic device. In some embodiments, the one or more nucleic acid molecules comprise DNA or ribonucleic nucleic acid (RNA) molecules. In some embodiments, the DNA is fragmented and single-stranded DNA. In some embodiments, the DNA is single-stranded DNA. In some embodiments, the RNA molecules comprise messenger RNA (mRNA) or microRNA (miRNA). In some embodiments, the RNA molecules comprise mRNA. In some embodiments, the one or more nucleic acid molecule capture probes comprise a sequence configured to couple to the one or more nucleic acid molecules. In some embodiments, the sequence configured to couple to the one or more nucleic acid molecules comprises a poly-T sequence, a randomer, a sequence complementary to at least a subset of the one or more nucleic acid molecules, or any combination thereof. In some embodiments, the microfluidic device is a well, bead, or a fluidic channel. In some embodiments, the fluidic channel is a flow cell. In some embodiments, the microfluidic device is not a bead. In some embodiments, the one or more nucleic acid molecule capture probes comprise one or more tags, wherein a tag comprises a cell-specific or spatial location-specific identifier sequence and optionally a unique molecular identifier (UMI) sequence. In some embodiments, the amplifying comprises solid-supported amplification. In some embodiments, the solid-supported amplification is bridge amplification. In some embodiments, the one or more nucleic acid molecules are derived from a single cell or biological tissue. In some embodiments, the method occurs in a gel matrix, wherein the gel matrix is adjacent to the solid support.

### Multi-Tiered Systems

Also provided herein are systems for analyzing a biological component comprising at least a flow channel (e.g., a first or upper layer) and an analysis channel (e.g., a second or lower layer). The system may comprise a fluidic device including a flow channel, an analysis channel, and a layer or wall disposed between the flow channel and the analysis channel. The system may include at least one energy source in communication with the fluidic device, as described herein. The analysis channel may be disposed adjacent to the flow channel, where at least one flow inhibition element may be disposed within the flow channel to inhibit or stop flow of the biological component in the flow channel. The layer disposed between the flow channel and the analysis channel may comprise at least one sealable aperture disposed at or adjacent to the at least one flow inhibition element. One or more biological components may be stopped or trapped adjacent to the sealable aperture. The at least one sealable aperture may be configured to allow passage of the one or more biological components. For example, the sealable aperture may be configured to allow passage of the one or more biological components from the flow channel to the analysis channel. The at least one energy source may be in communication with the analysis channel. Furthermore, the at least one energy source may form, or be configured to form, a polymer matrix within the analysis channel.

As described herein, in some embodiments, the fluidic device may comprise a microfluidic device or a nanofluidic device. In certain embodiments, the fluidic device may be used for nucleic acid sequencing. In some cases, the fluidic device may comprise a nucleic acid sequencing flow cell. In other cases, sequencing may comprise short-read sequencing, nanopore sequencing, sequencing by synthesis, sequencing by in situ hybridization, sequencing through collection of any optical readouts, or any other suitable method of sequencing.

As described herein, the biological component may comprise a cell, a cell lysate, a nucleic acid, a microbiome, a protein, a mixture of cells, a spatially-linked biological component, a metabolite, a combination thereof, or any other suitable biological component. In some cases, the mixture of cells may comprise two or more different cell types. For example, the mixture of cells may comprise a first cell type and a second cell type. In some cases, the mixture of cells may comprise 2, 3, 4, 5, 6, 7, 8, 9, 10, or more cell types. A cell may be a mammalian cell (e.g., a human cell), a fungal cell, a bacterial cell, a tumor spheroid, a combination thereof, or any other suitable cell. In some cases, the biological component may comprise a tumor spheroid or a spatially-linked biological component (or sample).

In some cases, the nucleic acid may comprise at least 100 bases or base pairs. In certain embodiments, a nucleic acid comprises a DNA or an RNA. The DNA may be at least 100 bp long. In some embodiments, the DNA may include at least 50 bp, 100 bp, 200 bp, 300 bp, 400 bp, 500 bp, 600 bp, 700 bp, 800 bp, 900 bp, 1,000 bp, 10 kilo base pairs (kbp), 100 kbp, 1 mega base pair (Mbp), 100 Mbp, 1 giga base pair (Gbp), 10 Gbp, 100 Gbp, or more base pairs. The biological component may comprise a DNA molecule that comprises any number of base pairs in between the mentioned numbers herein. For example, the DNA may comprise from 50 bp to 1,000 bp, 300 bp to 10 kbp, or 1,000 bp to 10 Gbp. The RNA may be dsRNA. The dsRNA may comprise at least 50 bp, 100 bp, 200 bp, 300 bp, 400 bp, 500 bp, 600 bp, 700 bp, 800 bp, 900 bp, 1,000 bp, 10 kbp, or 100 kbp. The biological component may comprise a dsRNA molecule that includes any number of base pairs in between the mentioned numbers herein. For example, the dsRNA may comprise from 50 bp to 1,000 bp, 300 bp to 10 kbp, or 1,000 bp to 100 kbp. The RNA may be ssRNA. The ssRNA may comprise at least 50 nucleotides to 100,000 nt. The ssRNA may comprise from 50 nt to 100 nt, 50 nt to 1,000 nt, 50 nt to 10,000 nt, 50 nt to 100,000 nt, 100 nt to 1,000 nt, 100 nt to 10,000 nt, 100 nt to 100,000 nt, 1,000 nt to 10,000 nt, 1,000 nt to 100,000 nt, or 10,000 nt to 100,000 nt. In some cases, the ssRNA may be less than 50 nucleotides long. The ssRNA may be more than 100,000 nucleotides long.

In some embodiments, the flow channel or a portion thereof may be parallel, or substantially parallel, with the analysis channel or at least a portion thereof. In some embodiments, the flow channel may be removably couplable to the analysis channel. For example, a user may remove the flow channel from the analysis channel. Accordingly, a portion of the fluidic device comprising the analysis channel may be used to conduct various analyses or experiments. With the portion of the fluidic device comprising the flow channel removed, the portion of the fluidic device comprising the analysis channel may be more accessible, e.g., to detectors, cameras, or other devices for analyzing the biological components within the analysis channel.

In some cases, the analysis channel may include polymer matrix structures for capturing or trapping a biological component or a molecule or compound produced by the biological component (e.g., prior to introduction of the biological components into the fluidic device). For example, a user may obtain an analysis channel that includes polymer matrix structures. That is, the user may not form the polymer matrix structures. In various cases, the analysis channel may be configured to include polymer matrix structures for capturing or trapping a biological component or a molecule or compound produced by the biological component. For example, in such embodiments, subsequent to introduction of the biological components into the fluidic device and the analysis channel, one or more polymer matrix structures may be formed in the analysis channel. The analysis channel may be configured for a screening process, a library preparation, or another suitable process. In some embodiments, the screening process may be for drug screening, antibiotic screening, culture conditions screening, or CRISPR screening. In certain cases, a plurality of samples may be placed into a plurality of channels. The plurality of samples may be screened against a variety of conditions in other signal-containing channels.

The sealable aperture may be configured to transition from a sealed state to an open state. For example, a sealable aperture may comprise a heat sensitive polymer that can melt, for example, upon receiving heat and render the sealable aperture open. In some cases, the passage of the biological component through the sealable aperture may be inhibited in the sealed state. In certain cases, the passage of the biological component through the sealable aperture may be allowed in the open state. In some cases, the sealable aperture may be sealed with an agarose gel, a temperature-soluble polymer, an N-isopropylacrylamide (NIPAAm) polymer, a wax compound, an alginate, or any other suitable compound or material.

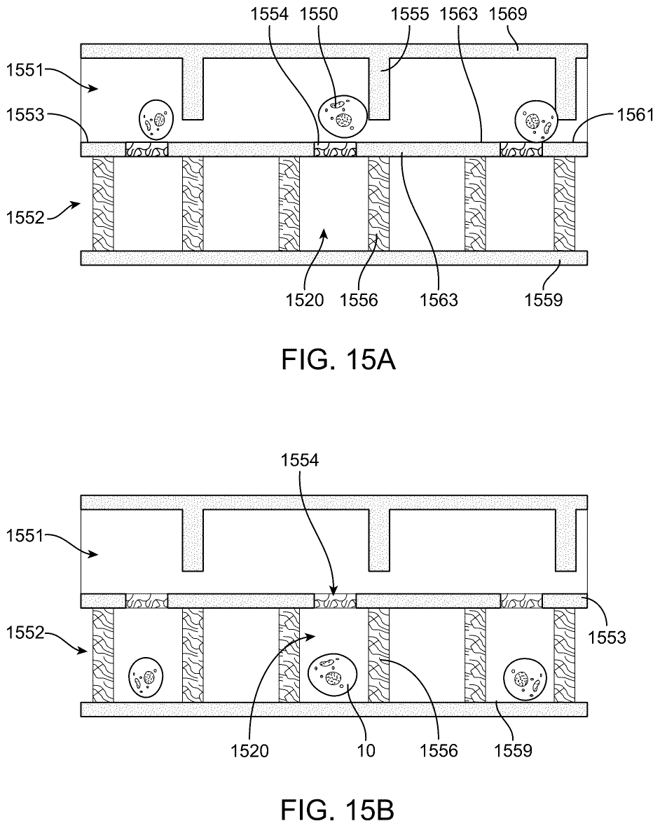

FIG. 15A and FIG. 15B show a portion of a fluidic device configured to trap a biological component 50. The fluidic device may comprise a flow channel or chamber 1551, an analysis channel or chamber 1552, and a layer or wall 1553 disposed between at least a portion of the flow channel 1551 and the analysis channel 1552. The layer 1553 may comprise one or more sealable apertures or openings 1554. Additionally, one or more flow inhabitation elements 1555 may inhibit or prevent, or be configured to inhibit or prevent, the biological component 50 from flowing along the flow channel 1511. A flow inhibition element 1555 may be configured to stop or trap the biological component 50 adjacent to a sealable aperture 1554. As described herein, the sealable aperture 1514 may be configured to transition from a sealed state (e.g., a closed state) or configuration to an open state or configuration. FIG. 15A illustrates an example of the sealable aperture 1514 in a sealed state. FIG. 15B illustrates an example of the sealable aperture 1554 in an open state. Upon transitioning to a sealed state to an open state, the sealable aperture 1554 may allow or permit passage of the biological component 50 from at least a portion of the flow channel 1551 to at least a portion of the analysis channel 1552. In certain instances, the analysis channel 1552 may be placed, or configured to be placed, below the flow channel 1551 to allow the biological component 50 to be transferred to the analysis channel 1552 from the flow channel 1551 by a force provided (e.g., via gravity, high pressure pulse by pressurizing a flow in the flow channel, and generating negative pressure in the analysis channel). In some embodiments, the fluidic device may be spun or centrifuged to disposed the one or more biological components from the flow channel to the analysis channel. Reagents can be disposed or passed through at least a portion of the analysis channel 1552, for example, to conduct analyses or experiments are provided herein.

As shown in FIG. 15A, the flow inhibition element 1555 may be disposed within at least a portion of the flow channel 1551 to inhibit or prevent flow of a biological component (e.g., biological component 50) in the flow channel 1551. The flow inhibition element 1555 may be configured to capture or trap the biological component 50 in at least a portion of the flow channel 1551. In some cases, the flow inhibition element 1555 may extend from a surface (e.g., surface 1569) of the flow channel 1551. In some cases, the surface 1569 may be disposed opposite of a flow channel surface 1561, which is adjacent to the layer 1553.

In various cases, the analysis channel 1552 may comprise a surface 1559 disposed opposite of the analysis channel surface 1563, which is adjacent to or a surface of the layer 1553. The analysis channel 1552 may comprise one or more polymer matrices 1556. The analysis channel 1552 may comprise one or more polymer precursors. For example, one or more polymer precursors may be disposed in at least a portion of the analysis channel 1552. The one or more polymer matrices 1556 may be formed using an energy source which provides energy to the one or more polymer precursors in the analysis channel 1502. The energy source may be in optical communication, electrochemical communication, electromagnetic communication, thermal communication, or microwave communication with the fluidic device or the analysis channel 1552. In some cases, the energy source may be a light generating device, a heat generating device, an electrochemical generating device, an electrode, a microwave device, or a combination thereof. The energy source may selectively provide energy to the analysis channel 1552 to form polymer matrices at predefined locations. A spatial energy modulating element may be used to selectively provide energy to the analysis channel 1552.

In some cases, the spatial energy modulating element may comprise a photolithographic mask, a DMD system, or other suitable mask. The one or more polymer matrices 1556 may be formed before the sealable aperture 1554 transitions to an open state (e.g., as shown in FIG. 15A). For example, a polymer matrix may be formed and aligned with the sealable aperture such that the biological component 1550 held by the inhibition element 1555 may directed (e.g., fall by gravity or by fluid pressure) into a compartment 1520 when the sealable aperture 1554 is rendered open. The one or more polymer matrices 1556 may be formed after the sealable aperture 1554 transitions to an open state (e.g., as shown in FIG. 15B). The one or more polymer matrices 1556 may form an analysis chamber or compartment 1520, as described herein.

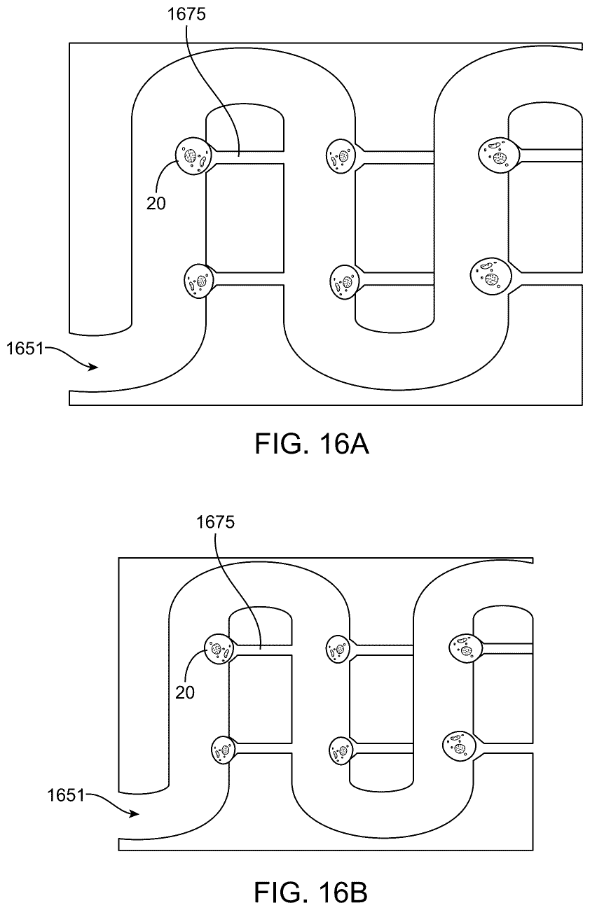

FIG. 16A and FIG. 16B show a top view of a fluidic device. A flow inhibition channel 1675 may be configured to inhibit a biological component 20 from flowing along a flow channel 1651. Flow of a fluid (e.g., a fluid including the biological component) through the flow channel 651 and the flow inhibition channel 1651 may cause the biological component 20 to be trapped or stopped at an opening of the flow inhibition channel 1675 as depicted in FIG. 16A. As illustrated, a dimension (e.g., a width) of the flow inhibition channel 1675 may be too small or narrow to allow or permit passage of the biological component 20 through the flow inhibition channel 1675. As shown in FIG. 16B, a polymer matrix 1676 may be formed on or adjacent to (e.g., surrounding) the biological component 20. In some cases, the polymer matrix may surround at least a portion of the biological component. The fluidic device of FIG. 16A and FIG. 16B may be a single-layer fluidic device. That is, the polymer matrix may be formed in the flow channel 1651. As illustrated, a path of the flow channel 1651 may be circuitous. For example, the flow channel 1651 may include one or more curves. In some embodiments, the path of the flow channel may be straight, substantially straight, in a zig-zag pattern, or any other suitable shape.

In certain embodiments, the fluidic device of FIG. 16A and FIG. 16B may comprise two or more layers. For example, the fluidic device may include a flow channel and an analysis channel (similar to the system shown in FIG. 15A and FIG. 15B). Further, a sealable aperture may be disposed at or adjacent to a portion of a flow inhibition channel. In such embodiments, the biological component may be transferred into the analysis channel (e.g., disposed adjacent to or below the flow channel) through a sealable aperture, as described herein. In some cases, the analysis channel may receive two or more biological components. For example, the analysis channel may receive 2, 3, 4, 5, 6, 7, 8, 9, 10, or more biological components.

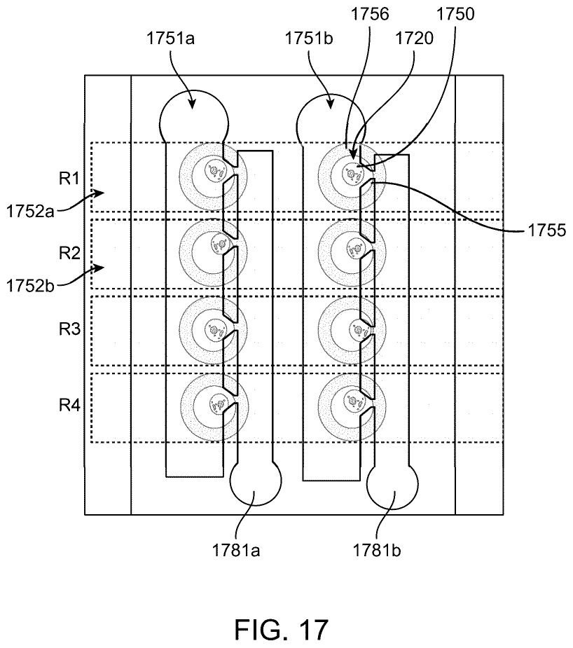

FIG. 17 illustrates an example of a fluidic device including, or configured for, a plurality of reagents and/or analytes (R1, R2, R3, and R4). The fluidic device may comprise a first flow channel 1751a to receive one or more biological components from a first sample. The first flow channel 1751a may allow or permit flow or passage of one or more biological components from the first sample. Further, the first flow channel 1751a may allow or permit flow or passage of one or more polymer precursors. The fluidic device may comprise a second flow channel 1751b to receive one or more biological components from a second sample. The second flow channel 1751b may allow or permit flow or passage of one or more biological components from the second sample. Further, the second flow channel 1751b may allow or permit flow or passage of one or more biological components from the second sample.

The first flow channel 1751a and/or the second flow channel 1751b may comprise a plurality of inhibition elements (e.g., inhibition element 1755). A biological component (e.g., biological component 50) may be trapped or localized by the inhibition element 1755. As described herein, the first flow channel 1751a and/or the second flow channel 1751b may comprise one or more sealable apertures disposed at or adjacent to the one or more inhibition elements 1755 that can be opened (e.g., transitioned from a sealed state to an open state) to allow the biological component to move into a first analysis channel 1752a or a second analysis channel 1752b. The first and second flow channels 1751a and 1751b may be disposed above the first and second analysis channels 1752a and 1752b (e.g., in an upper layer and a lower layer similar to the fluidic device illustrated in FIG. 15A and FIG. 15B). A polymer matrix 1756 may be formed surrounding the biological component 50. The polymer matrix 1756 may partially surround the biological component 50. The polymer matrix 1756 may form a compartment or an analysis chamber 1720 to localize the biological component 50 within at least a portion of an analysis channel (e.g., analysis channels 1751a and 1751b).

The first analysis channel 1752a may comprise one or more reagents and/or analytes that are different from the one or more reagents and/or analytes in the second analysis channel 1752b. The first analysis channel 1752a may comprise one or more reagents and/or analytes that are the same as the one or more reagents and/or analytes in the second analysis channel 1752b. In some cases, the fluidic device may include at least 2, 3, 4, 5, 6, 7, 8, 9, 10, 15, 25, 50, or more flow channels In certain cases, the fluidic device may include at least 2, 3, 4, 5, 6, 7, 8, 9, 10, 15, 25, 50, or more analysis channels. The fluidic device may analyze a plurality of biological components in parallel. The plurality of biological components may be exposed to one or more different reagents and/or analytes, as provided herein. Such a configuration (e.g., as shown in FIG. 17) may allow a plurality of biological components in one or more samples to be analyzed under various conditions provided by the different reagents and/or analytes. The fluidic device, shown in FIG. 17, may be used for a screening process. The screening process may be for drug screening, antibiotic screening, culture conditions screening, or CRISPR screening. The screening process may be performed in combinatorial manner. For example, a plurality of samples may be loaded in a plurality of flow channels (e.g., in parallel) which may be screened against a plurality of conditions in the plurality of analysis channels.

The first and/or the second samples may be homogenous or heterogenous. For example, the one or more biological components in the first sample may be the same or different. The first sample may be different from the second sample. In some cases, a biological component may be released from a compartment or analysis chamber 1720 by selectively degrading a polymer matrix, as described herein. In other words, a polymer matrix may be degraded “on demand” (e.g., by a user or as directed by a computer). In various embodiments, the degradation may be achieved through the use of localized stimuli. In certain embodiments, the degradation may be achieved through the use of heat, light, electrochemical reactions, or some combination thereof. The released biological component may be collected using an outlet channel (e.g., outlet channel 1781a or 1781b).

As described in reference to the fluidic device of FIG. 15A and FIG. 15B, a layer may be disposed between the flow channels 1751a and 1751b and the analysis channels 1752a and 1752b. The analysis channel surface adjacent to the layer (e.g., similar to surface 1561 shown in FIG. 15A), the analysis channel surface opposite of the layer (e.g., similar to surface 1559 shown in FIG. 15A), or both may comprise one or more barcodes, as described herein.

In some cases, the channels and/or the analysis may comprise molecules in addition to, or instead of, the one or more barcodes. For example, any of the surfaces of the one or more channels and/or analysis channels may comprise an optical (e.g., fluorescence), mechanical, electrical or biochemical sensing element or sensor. The sensing element may comprise a fluorescent tag, an enzyme, a primer, an oligonucleotide, or a sensor molecule (e.g., a biochemical sensor molecule). The sensing element may be used to detect and/or measure a pH, an oxygen concentration, a CO₂ concentration, or any other suitable variable. The sensing element may detect and/or measure a parameter locally. For example, the sensing element may detect and/or measure a pH, an oxygen concentration, or a CO₂ concentration within a compartment (e.g., a polymer matrix shell cylinder) surrounding the biological component.

### Hydrogel Chambers

In some embodiments, hydrogel chambers may be used as diffusivity modifiers that limit the distance predetermined cellular nucleic acid molecules may travel away from a lysed cell. A wide variety of photosynthesizable gels may be used in connection with the invention. In some embodiments, hydrogels are used with the invention, in particular because of their compatibility with living cells and the versatility of formulating gels with desired properties including, but not limited to, porosity (which in large part determines what is contained and what is passed by a gel (or polymer matrix) wall, degradability, mechanical strength, ease and speed of synthesis, and the like).

Porosity. In some embodiments, hydrogel porosity is selected to permit passage of selected reagents while at the same time preventing the passage of other reagents. In some embodiments, hydrogel porosity is selected to prevent the passage of biological cells but to permit the passage of reagents, including proteins, such as polymerases. In some embodiments, reagents permeable to a polymer matrix wall comprise lysozyme, proteinase K, random hexamers, polymerases, transposases, ligases, deoxynucleotide triphosphates, buffers, cell culture media, or divalent cations. In some embodiments, the at least one polymer matrix comprises pores that are sized to allow diffusion of a reagent through the at least one polymer matrix but are too small to allow DNA or RNA for analysis to traverse the pores (having a size of greater than 100 nucleotides or basepairs, or greater than 300 nucleotides or basepairs). In some embodiments, crosslinking the polymer chains of the hydrogel structure forms a hydrogel matrix having pores (i.e., a porous hydrogel matrix). In some versions, the size of the pores in the hydrogel structures may be regulated or tuned and may be formulated to encapsulate sufficiently large genetic material (e.g., of greater than about 300 base pairs), but to allow smaller materials, such as reagents, or smaller sized nucleic acids (e.g., of less than about 50 base pairs), such as primers, to pass through the pores, thereby passing in and out of the hydrogel structures. In some embodiments, the hydrogels may comprise a pore size having a diameter sufficient to allow diffusion of the above-listed reagents through the structure while retaining the nucleic acid molecules greater than 500 nucleotides or basepairs in length. In some embodiments, the pores have a diameter of from about 10 nm to about 100 nm. In some embodiments, the pore size of the hydrogel structures may be determined by varying the ratio of the concentrations of polymer precursors to the concentration of crosslinkers, varying pH, salt concentrations, temperature, light intensity, and the like, by routine experimentation. In some embodiments, the average diameter of pores of a polymer matrix wall prevent passage of molecules having a molecular weight of 25 kiloDaltons (kDa) or greater; or having a molecular weight of 50 kDa or greater; or having a molecular weight of 75 kDa or greater; or having a molecular weight of 100 kDa or greater; or having a molecular weight of 150 kDa or greater.

In some embodiments, DNA or RNA retained have lengths that are sequenceable using conventional sequencing-by-synthesis techniques. For example, such DNA or RNA comprise at least 50 nucleotides, or in some embodiments, at least 100 nucleotides. In some embodiments, the pores may have an average diameter from 5 nm to 100 nm. In some embodiments, the pores may have an average diameter from 5 nm to 10 nm, 10 nm to 20 nm, 20 nm to 30 nm, 30 nm to 40 nm, 50 nm to 60 nm, 60 nm to 70 nm, 70 nm to 80 nm, 80 nm to 90 nm, 90 nm to 100 nm. In some embodiments, the pores may have an average diameter larger than 100 nm. In some embodiments, the pores may have an average diameter smaller than 5 nm. The reagent may comprise an enzyme or a primer having a size of less than 50 base pairs (bp). A primer may comprise a single-stranded DNA (ssDNA). In some embodiments, a primer may have a size from 5 bp to 50 bp. In some embodiments, a primer may have a size from 5 bp to 10 bp, 10 bp to 20 bp, from 20 bp to 30 bp, 30 bp to 40 bp, or 40 bp to 50 bp. In some embodiments, a primer may have a size of more than 50 bp. In certain cases, a primer may have a size of less than 5 bp. In some embodiments, the pores may have a diameter from 5 nm to 100 nm. In some embodiments, the pores may have a diameter from 5 nm to 10 nm, 10 nm to 20 nm, 20 nm to 30 nm, 30 nm to 40 nm, 50 nm to 60 nm, 60 nm to 70 nm, 70 nm to 80 nm, 80 nm to 90 nm, 90 nm to 100 nm. In some embodiments, the pores may have a diameter larger than 100 nm. In some embodiments, the pores may have an average diameter smaller than 5 nm. The polymer matrix may have a pore size of about 5 nanometers (nm) to about 100 nm. The polymer matrix may have a pore size of about 5 nm to about 10 nm, about 5 nm to about 20 nm, about 5 nm to about 30 nm, about 5 nm to about 40 nm, about 5 nm to about 50 nm, about 5 nm to about 60 nm, about 5 nm to about 70 nm, about 5 nm to about 80 nm, about 5 nm to about 90 nm, about 5 nm to about 100 nm, about 5 nm to about 110 nm, about 10 nm to about 20 nm, about 10 nm to about 30 nm, about IO nm to about 40 nm, about 10 nm to about 50 nm, about 10 nm to about 60 nm, about 10 nm to about 70 nm, about 10 nm to about 80 nm, about 10 nm to about 90 nm, about 10 nm to about I 00 nm, about 10 nm to about 110 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 20 nm to about 110 nm, about 30 nm to about 40 nm, about 30 nm to about 50 nm, about 30 nm to about 60 nm, about 30 nm to about 70 nm, about 30 nm to about 80 nm, about 30 nm to about 90 nm, about 30 nm to about I 00 nm, about 30 nm to about 110 nm, about 40 nm to about 50 nm, about 40 nm to about 60 nm, about 40 nm to about 70 nm, about 40 nm to about 80 nm, about 40 nm to about 90 nm, about 40 nm to about I 00 nm, about 40 nm to about 110 nm, about 50 nm to about 60 nm, about 50 nm to about 70 nm, about 50 nm to about 80 nm, about 50 nm to about 90 nm, about 50 nm to about 100 nm, about 50 nm to about 110 nm, about 60 nm to about 70 nm, about 60 nm to about 80 nm, about 60 nm to about 90 nm, about 60 nm to about 100 nm, about 60 nm to about 110 nm, about 70 nm to about 80 nm, about 70 nm to about 90 nm, about 70 nm to about 100 nm, about 70 nm to about 110 nm, about 80 nm to about 90 nm, about 80 nm to about 100 nm, about 80 nm to about 110 nm, about 90 nm to about 100 nm, about 90 nm to about 110 nm, or about 100 nm to about 110 nm. The polymer matrix may have a pore size of about 5 nm, about 10 nm, about 20 nm, about 30 nm, about 40 nm, about 50 nm, about 60 nm, about 70 nm, about 80 nm, about 90 nm, about 100 nm, or about 110 nm. The polymer matrix may have a pore size of at least about 5 nm, about 10 nm, about 20 nm, about 30 nm, about 40 nm, about 50 nm, about 60 nm, about 70 nm, about 80 nm, about 90 nm, about 100 nm, or less. The polymer matrix may have a pore size of at most about 10 nm, about 20 nm, about 30 nm, about 40 nm, about 50 nm, about 60 nm, about 70 nm, about 80 nm, about 90 nm, about 100 nm, about 110 nm, or more.

Modulation of Porosity. The pore size in the polymer matrix may be modulated using a chemical reagent, or by applying heat, electrical field, light, or another suitable stimulus. In other words, the polymer matrix may comprise tunable properties (e.g., the pore size) In some cases, the polymer matrix may comprise a thermoresponsive or temperature-responsive polymer. A thermoresponsive polymer (e.g., poly(N-isopropylacrylamide) (NIPAAM)) may phase separate from a solution upon heating or upon cooling (e.g., polymer showing lower critical solution temperature (LCST) or upper critical solution temperature (UCST). The polymer matrix may comprise polymer which may collapse at high temperature in order to, for example, control the pore size of the hydrogel or polymer matrix. Non-limiting examples of thermoresponsive polymers that may be used to form hydrogel/polymer matrix with tunable properties may include Poly(N-vinyl caprolactam), Poly(N-ethyl oxazoline), Poly(methyl vinyl ether), Poly(acrylic acid-coacrylamide), or a combination thereof. A change in temperature may enlarge or contract average pore size in the polymer matrix to allow selected molecules, such as a nucleic acid molecule, a protein, or any biomolecule or molecule smaller than the adjusted pore size to be released from a hydrogel chamber.

Size and Shape of Hydrogel Chambers. In some embodiments, a polymer matrix wall of a chamber inhibits passage of a predetermined component, such as a mammalian cell, genomic DNA, larger polynucleotides (e.g. mRNA greater than 200 ribonucleotides, or greater than 300 ribonucleotides, or 500 ribonucleotides), or the like. In some embodiments, a polymer matrix wall extends from the first surface to a second surface (parallel to the first surface) to form a chamber within a channel. In some embodiments, a chamber has polymer matrix walls and an interior, with an interior area, which is the area of the surface enclosed by the chamber. In some embodiments, the interior of a chamber is sized for enclosing a cell. For example, such chamber may comprise a cylindrical shell or a polygon shell, comprising an inner space, or interior and a polymer matrix wall. In some embodiments, such chambers have annular-like cross-sections. As used herein, the term “annular-like cross-section” means a cross-section topologically equivalent to an annulus. In some embodiments, the inner space, or interior, of a chamber has a diameter in the range of from 1 μm to 500 μm and a volume in the range of from 1 pico liter to 200 nano liters, or from 100 pico liters to 100 nano liters, or from 100 picoliters to 10 nano liters. In some embodiments, hydrogel chamber enclose a surface area in the range of from 5 μm² to 1×10⁶ μm², or in the range of from 400 μm² to 7×10⁵ μm². In some embodiments, the polymer matrix wall has a thickness of at least 1 μm (micrometer). In some embodiments, the height of a chamber with an annular-like cross section have a value in the range of from 10 μm to 500 μm, or in the range of from 50 μm to 250 μm. In some embodiments, a polymer matrix wall having an annular-like cross-section has an aspect ratio (i.e., height/width) of 1 or less. In some embodiments, aspect ratio and polymer matrix wall thickness are selected to maximize chamber stability against forces, such as reagent flow through the channel, washings, and the like. In some embodiments, the at least one polymer matrix wall is a hydrogel wall. In some embodiments, the at least one polymer matrix is degradable. In some embodiments, the degradation of the at least one polymer matrix is “on demand.” In some embodiments, chambers in a channel are non-contiguous. In some embodiments, chambers in a channel may be contiguous with adjacent chambers. In some embodiments, chambers may share polymer matrix walls with one another. In some embodiments, chambers may be synthesized with slits or other orifaces large enough to permit passage of certain components, e.g. beads, but small enough to prevent passage of other components, e.g. cells.

Hydrogel Compositions. In some embodiments, a channel of a fluidic device of a system of the invention comprises one or more polymer precursors for forming chambers. In some embodiments, the one or more polymer precursors comprise hydrogel precursors. Such precursors may be selected from a wide variety of compounds including, but not limited to, polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetraacrylate, or combinations or mixtures thereof. In some embodiments, the hydrogel comprises an enzymatically degradable hydrogel, PEGthiol/PEG-acrylate, acrylamide/N,N′-bis(acryloyl)cystamine (BACy), or PEG/PPO. In some embodiments, the following precursors and crosslinker may be used to form chambers with degradable polymer matrix (hydrogel) walls. Polymer precursors may be formed by using any hydrogel precursor and crosslinkers of Table 1A (columns 1 and 3, respectively). The resulting polymer matrices may be degraded with the indicated degradation agents in Table 1A (column 4).

TABLE 1A

| | | | Degradation |
|---|---|---|---|
| Precursors | Hydrogels | Crosslinkers | Agents |
| Acrylamide | Polyacryl- | Bis-acryloyl cystamine | DTT/TCEP/THP |
| | amide | (structure 1) | |
| PEG-based | PEG | Bis(2-methacryloly) | DTT/TCEP/THP |
| acryloyl | | oxyethyl disulfide | |
| | | (structure 2) | |
| Dextran- | Dextran | N,N′-(1,2-Dihydroxyl- | NaIO4 |
| based | | ethylene)bis-acrylamide | |
| acryloyl | | (structure 3) | |
| Poly- | Poly- | Structure 4 | NaOH, ethanolamine |
| saccharide- | saccharide | | DTT/TCEP/THP |
| base acryloyl | | | |
| Gelatin-base | Gelatin | Structure 5 | NaOH, ethanolamine, |
| acryloyl | | | nucleophilic bases |
| | | Structure 6 | NaOH, alkali, organic |
| | | | bases |
| | | Structure 7 | Acid |

TABLE 1B

| Structure | |
|---|---|
| Number | Formula |
| 1 | 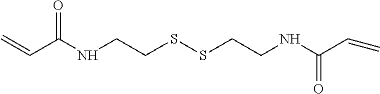 [SMILES: C=CC(=O)NCCSSCCNC(=O)C=C] |
| 2 | 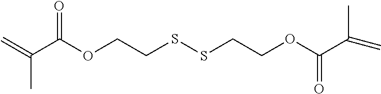 [SMILES: C=C(C)C(=O)OCCSSCCOC(=O)C(=C)C] |
| 3 | 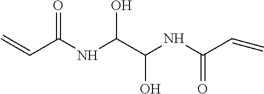 |
| 4 | 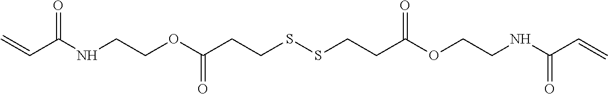 [SMILES: C=CC(=O)NCCOC(=O)CCSSCCC(=O)OCCNC(=O)C=C] |
| 5 | 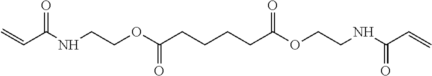 [SMILES: C=CC(=O)NCCOC(=O)CCCCC(=O)OCCNC(=O)C=C] |
| 6 | 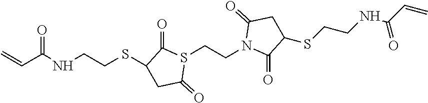 |
| 7 | 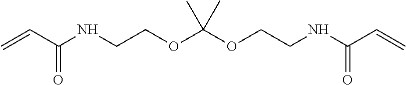 [SMILES: C=CC(=O)NCCOC(C)(C)OCCNC(=O)C=C] |

Hydrogel Degradation. In some embodiments, hydrogel chambers of the invention are degradable or depolymerizable either generally within a channel or “on demand” within a channel. Hydrogel chambers that are generally degradable are degraded by treatment with a degradation agent, or equivalently, a depolymerization agent that is exposed to all chambers within channel. Depolymerization agents may include, but are not limited to, heat, light, and/or chemical depolymerization reagents (also sometimes referred to a cleaving reagents or degradation reagents). In some embodiments, on demand degradation may be implemented using polymer precursors that permit photo-crosslinking and photo-degradation, for example, using different wavelengths for crosslinking and for degradation. For example, Eosin Y may be used for radical polymerization at defined regions using 500 nm wavelength, after which illumination at 380 nm can be used to cleave the cross linker. In other embodiments, photo-caged hydrogel cleaving reagents may be included in the formation of polymer matrix walls. For example, acid labile crosslinkers (such as esters, or the like) can be used to create the hydrogel and then UV light can be used to generate local acidic conditions which, in turn, degrades the hydrogel. In some embodiments, the at least one polymer matrix is degradable by at least one of: (i) contacting the at least one polymer matrix with a cleaving reagent; (ii) heating the at least one polymer matrix to at least 90° C.; or (iii) exposing the at least one polymer matrix to a wavelength of light that cleaves a photo-cleavable cross linker that cross links the polymer of the at least one polymer matrix. In some embodiments, the at least one polymer matrix comprises a hydrogel. In some embodiments, the cleaving reagent degrades the hydrogel. In some embodiments, the cleaving reagent comprises a reducing agent, an oxidative agent, an enzyme, a pH based cleaving reagent, or a combination thereof. In some embodiments, the cleaving reagent comprises dithiothreitol (DTT), tris(2-carboxyethyl)phosphine (TCEP), tris(3-hydroxypropyl)phosphine (THP), or a combination thereof. In some embodiments, the surface of the polymer matrix or hydro gel may be functionalized by coupling a functional group to the polymer matrix or hydrogel. Some nonlimiting examples of functional group may include a capture reagent (e.g., pyridinecarboxaldehyde (PCA)), an acrylamide, an agarose, a biotin, a streptavidin, a strep-tag II, a linker, a functional group comprising an aldehyde, a phosphate, a silicate, an ester, an acid, an amide, an aldehyde dithiolane, PEG, a thiol, an alkene, an alkyne, an azide, or a combination thereof. In some cases, the functionalized polymer matrix may be used to capture biomolecules inside a polymer matrix compartment formed adjacent to (e.g., around or on) the biological component. The biomolecule may be produced by the biological component (e.g., secretome from a cell). The functionalized surface of the polymer matrix inside the compartment may be used to capture reagents or molecules from outside the compartment. The functionalized surface may increase surface area covered by a reagent, a molecular sensor, or any molecule of interest (e.g., an antibody).

Partial Degradation. In some embodiments, existing polymer matrix walls may be partially degraded, e.g. to change porosity. In some embodiments, polymer precursors may include degradable beads that form part of, and are embedded in, the polymer matrix walls when synthesized, after which either on-demand or generally, may be degraded, thereby creating an increase in porosity.

Photosynthesis. In some embodiments, the generation of a polymer matrix within said fluidic device comprises exposing the one or more polymer precursors to an energy source. In some embodiments, the energy source is a light generating device. In some embodiments, the light generating device generates light at 350 nm to 800 nm. In some embodiments, the light generating device generates light at 350 nm to 600 nm. In some embodiments, the light generating device generates light at 350 nm to 450 nm. In some embodiments, the light generating device generates UV light. In some embodiments, the generation of a polymer matrix within said fluidic device is performed using a spatial light modulator (SLM) (i.e. a spatial energy modulation element that is capable of generating desired light intensity pattern spatially). In some embodiments, the SLM is a digital micromirror device (DMD). In some embodiments, the SLM is a laser beam steered using a galvanometer. In some embodiments, the SLM is liquid-crystal based.

### Sequencing, Barcodes, Genomic Fragments and Transcriptomes

Oligonucleotide labels, barcodes, genomic fragments, RNAs, including messenger RNAs and other polynucleotide targets, or nucleic acid molecules, may be sequenced by methods and systems of the invention. In some embodiments, capture elements, or capture probes, for this purpose include oligonucleotides attached to a surface, wherein such oligonucleotides comprise a sequence segment that is complementary to that of the nucleic acids to be captured, which may be (for example) a polyA segment of mRNA or an arbitrary sequence, or “handle,” segment adjacent to a barcode or oligonucleotide label. When sequencing operations are to be performed a surface is provided with such capture elements (such as oligonucleotides) and optionally surface primers for surface amplification of captured nucleic acids or derivatives thereof. Such capture oligonucleotides may be attached to a first surface by many chemistries known in the art, e.g. Integrated DNA Technologies brochure entitled “Strategies for attaching oligonucleotides to solid supports,” (2014). A sequencing step may be performed on the surface adjacent to a lysed cell (“in situ” sequencing) or templates may be optionally amplified, released and eluted from the surface and sequenced on an external sequencing instrument (“external” sequencing). In the latter approach, capture elements may include a spatial barcode that provides surface position information, and permits externally determined sequences to be associated with particular locations on the surface, e.g. the site of individual chambers. In some embodiments, spatial barcodes are present in sufficiently high density such that each chamber covers an area of a surface that is uniquely associated with one or more spatial barcodes, and usually a single spatial barcode. In some embodiments, the preparation of polynucleotides for a sequencing operation takes place after the target templates (e.g. oligonucleotide label, mRNAs, genomic fragments) are released and captured by complementary sequences in the capture elements. A releasing step depends on the nature of the target templates. For example, oligonucleotide labels attached to antibodies by a disulfide linkage may be released by a reducing agent (which may be the same as a lysing reagent). mRNAs may be release by treating cells with conventional lysing reagents. Releasing genomic fragments may require lysing and pre-amplification steps. Lysing conditions may vary widely and may be based on the action of heat, detergent, protease, alkaline, or combinations of such factors. The following references provide guidance for selection of lysing reagents, or lysing buffers, for single-cell lysing conditions for mRNA and/or genomic DNA: Thronhill et al, Prenatal Diagnosis, 21: 490-497 (2001); Kim et al, Fertility and Sterility, 92: 814-818 (2009); Spencer et al, ISME Journal, 10: 427-436 (2016); Tamminen et al, Frontiers Microbiol. Methods, 6: article 195 (2015); and the like. Lysis conditions may include the following: 1) cells in H2O at 96° C. for 15 min, followed by 15 min at 10° C.; 2) 200 mM KOH, 50 mM dithiotheitol, heat to 65° C. for 10 min; 3) for 4 μL protease-based lysis buffer: 1 μL of 17 μM SDS combined with 3 μL of 125 μg/mL proteinase K, followed by incubation at 37° C. for 60 min, then 95° C. for 15 min (to inactivate the proteinase K); 4) for 10 μL of a detergent-based lysis buffer: 2 μL H2O, 2 μL 10 mM EDTA, 2 μL 250 mM dithiothreitol, 2 μL 0.5% N-laurylsarcosin salt solution; 5) 200 mM Tris pH7.5, 20 mM EDTA, 2% sarcoyl, 6% Ficoll.

In embodiments employing spatial barcodes on a surface, a wide variety of methods may be used to generated spatial barcodes including, but not limited to, the methods described in the following references which are incorporated by reference: Horgan et al, International patent publication WO2022/013094; Frisen et al, U.S. Pat. No. 9,593,365; Fan et al, U.S. patent publication US2019/0360121; Chen et al, bioRxiv (https://doi.org/10.1101/2021.01.17.427004); Cho et al, bioRxiv (https://doi.org/10.1101/2021.01.25.427807); Quan et al, Nature Biotechnology, 29(5): 449-453 (2011); Singh-Gasson et al, Nature Biotechnology, 17: 974-(1999); and the like.

### Systems and Instrumentation

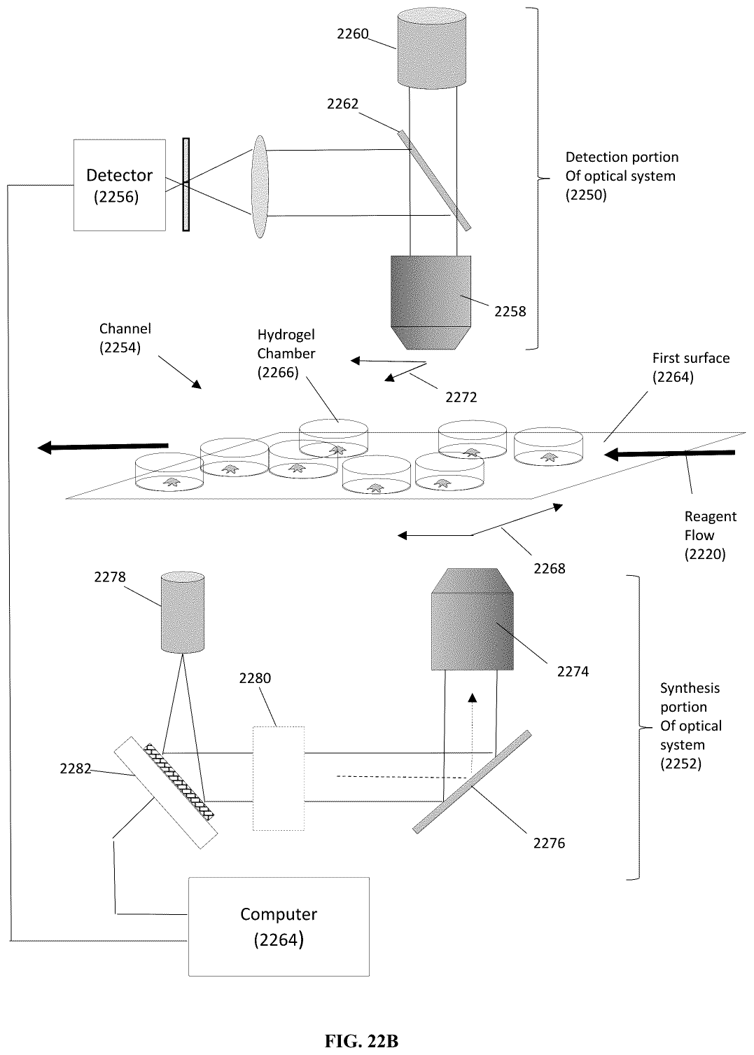

A system for carrying out embodiments (such as illustrated in FIGS. 2E-2F) employs gel barriers as diffusivity modifiers is illustrated in FIG. 22A. Flow cell (2200) is a component of a fluidic device that provides one or more channels and liquid handling components under programmable control for delivering beads and reagents to the channels. In this illustration, four channels (2202, 2204, 2206, and 2208) are shown, with blow-up view (2212) of segment (2210) of channel 2 (2204) shown below. In the abstracted view of flow cell (2200) of FIG. 22A, inlets, outlets and other features of the channels are not shown. On first surface (2214) of channel 2 (2204) a plurality of beads, e.g. (2218), are each enclosed by a hydrogel chamber, e.g. (2216). In some embodiments, the porosity of polymer matrix walls of the hydrogel chambers is selected to be impermeable to the beads, but permeable to reagents for forming spatial barcodes. Thus, reagents may be introduced to, and removed from, the interiors of the hydrogel chambers by flowing (2220) them through the channels, but beads are retained inside. Below blow-up (2212) of channel segment (2210) is shown an optical system (2221) for photosynthesizing hydrogel chambers at the locations of beads in the channels. One of ordinary skill in the art would recognize that optical systems with different configurations than those of FIGS. 22A and 22B may be employed for carrying out these functions. In some embodiments, one or more DMD-objective subsystems for synthesizing hydrogel structures may be employed to increase the speed of synthesis by synthesizing multiple structures simultaneously.

Returning to FIG. 22A, for photosynthesizing the hydrogel chambers, light source (2222) generates light beam (2223) of appropriate wavelength light (e.g. UV light) that passes through an appropriate photo-mask or beam-shaping or beam steering (Galvo) system for shaping a beam to synthesize a desired structure or structures in a channel. In some embodiments, a digital micromirror device (DMD)(2224) is employed, in other embodiments, a physical photo-mask may be employed. Chamber position, shape and polymer matrix wall thickness is determined at least in part from bead position information determined from images collected by detector (2232). Reflected light from DMD (2224) is shaped using conventional optics, e.g. collimating optics (2228), and is directed through objective lens system (2234) into channel 2 segment (2210). Objective (2234) and flow cell (2200) move relative to one another in the xy-directions (2236) to photosynthesize chambers at any position in any of the channels. In some embodiments, flow cell (2200) moves and optical system (2221) is stationary. In some embodiment, objective (2234) may also direct light beam (2227) from light source (2229) to targets, such as cells, on first surface (2214) and collect optical signals, such as fluorescent signals, from assays taking place on first surface (2214). Alternatively, optical signal collection may be carried out with a separate objective as shown if FIG. 22B. Information collected by detector (2232), or its counterpart in the embodiment of FIG. 22B, particularly cellular positions in their respective channels, is employed by computer (2238) and/or subsidiary controllers to direct DMD (2224) and translation devices controlling the relative positions of objective (2234) and flow cell (2200) to synthesize hydrogel chambers of the appropriate shape and size at the appropriate locations.

FIG. 22B illustrates an alternative optical system in which the detection portion (2250) of the optical system moves (2272) independently from the movement (2268) of the synthesis portion (2252) of the optical system. Detection portion (2250) of the optical system comprises detector (2256), objective (2258), light source (2260) and interconnecting optical elements, such as dichroic mirror (2262). As with the embodiment of FIG. 22A, detector (2256) is operationally associated with computer (2264) and the synthesis portion (2252) of the optic system to provide synthesis portion (2252) with bead position information. Computer (2264) and (2238) are also in operationally associated with stages and/or motors controlling the relative positions of the objectives of the optical systems and the position of the flow cell. In this embodiment, synthesis portion (2252) of the optical system is located on the other side of first surface (2264) from detection portion (2250). As with the embodiment of FIG. 22A, it comprises the conventional components objective (2274), mirror (2276), collimating optics (2280), DMD (2282) and light source (2278).

In some embodiments, beads, e.g. (2218) in FIG. 22A, are disposed randomly on first surface (2214). In alternative embodiments, first surface (2214) may comprise regularly spaced sites or features for capturing beads so that they are disposed substantially only on such sites or features on the first surface. For example, in some embodiments, such sites or features may be a rectilinear or a hexagonal array of spots.

In some embodiments, systems of the invention comprise (a) a channel comprising a first surface, a plurality of cells disposed on the first surface, and one or more polymer precursors; (b) a spatial energy modulating element in optical communication with the first surface; (c) a detector in optical communication with the first surface and in operable association with the spatial energy modulating element, the detector detecting each of the plurality of cells and determining a position thereof on the first surface; and (d) a plurality of gel chambers each gel chamber enclosing a single cell of the plurality of cells wherein the gel chambers are synthesized by projecting light into the channel with the spatial energy modulating element such that the projected light causes cross-linking of the one or more polymer precursors to form polymer matrix walls of the chambers, wherein the positions of the synthesized chambers are determined by the positions of cells enclosed thereby identified by the detector. It is understood that the term “detector” as used herein may include, but not be limited by, a microscope element that collects and optionally magnifies an image of a portion of a channel and an image analysis element that comprises software for identifying cells, cellular features, chambers, and other objects, for storing such information as well as associated position information. A computer element uses such information generated by a detector together with user input to generate commands to other elements, such as, the spatial energy modulating element to carry out a variety of functions including, but not limited to, synthesizing chambers, “on-demand” degrading of chambers, photo-lysing cells, and the like. Configurations of such embodiments are illustrated in FIGS. 22A-22B which are described above. In some embodiments, a channel of a fluidic device further comprises a second surface wherein said first surface and the second surface are disposed opposite one another across the channel, and wherein the polymer matrix walls of the chambers extend from the first surface to the second surface to form chambers each having an interior. In some embodiments, chambers in a channel each enclose a single cell. In some embodiments both the first wall and the second wall are made of optically transmissive materials, such as, glass, plastic, or the like, and are positioned so that the first surface and second surface are substantially parallel to one another. the perpendicular distance between a first surface and a second surface may be in the range of from 10 μm to 500 μm, or in the range of from 50 μm to 250 m. In some embodiments, the perpendicular distance between a first surface and a second surface may be in the range of from twice the average size of the cells to be analyzed to five times the average size of the cells to be analyzed.

In some embodiments, the first surface may comprise capture elements for capturing cells at predetermined locations. For example, capture elements may include, but are not limited to, capture antibodies specific for all or a subpopulation of cells. Capture elements may also include, but not be limited to, non-specific capture materials, such as, polylysine, fibronectin, treated plastics (e.g. Maxysorb™ plastic, ThermoFisher), and the like. In some embodiments, such cellular capture moieties (for example, antibodies) may be restricted to spots or reaction sites arrayed in a regular pattern on the first surface; thus, cells captured at such reaction sites may be disposed on the first surface in a regular pattern that may be more efficiently than a random disposition for chamber synthesis and/or optical signal detection. Guidance for providing surfaces with cellular capture antibodies may be found in the following references: Zhu et al, Analytica Chemica Acta, 608: 186-196 (2008); Sekine et al, J. Immunol. Methods, 313(1-2): 96-109 (2006); and the like. In some embodiments, such reaction sites or spots have diameters in the range of from 5-500 m or in the range of from 10-1000 m. In some embodiments, such spots or reaction sites are arranged in a rectilinear array, or are arranged in a hexagonal array. In some embodiments, such arrays of such spots or reaction sites have a density in the range of from 10 to 2500 sites/mm², or from 10 to 1000 sites/mm², or from 10 to 500 sites/mm², or from 10 to 100 sites/mm².

Spatial energy modulating elements using light energy for polymerization may comprise physical photomasks or virtual photomask, such as, a digital micromirror device (DMD). The following references, which are hereby incorporated by reference, provide guidance in selecting and operating a DMD for photopolymering gels: Chung et al, U.S. patent Ser. No. 10/464,307; Hribar et al, U.S. patent Ser. No. 10/351,819; Das et al, U.S. Pat. No. 9,561,622; Huang et al, Biomicrofluidics, 5: 034109 (2011); and the like.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1

### RNA Sequencing Employing the Method Described Herein

An experiment with a P7 or a P5 adapted library from RNA transcript is performed. RNA transcripts are generated from a plasmid containing Green Fluorescent Protein (GFP). The pMA-T based plasmid contains P5, a T7 polymerase promoter, a start codon, a His tag, a FLAG tag, the GFP sequence, a TAA stop codon, a T7 terminator, and P7. The RNA transcript can extend from the promoter sequence to the T7 termination sequence. However, the T7 terminator does not stop transcription completely and so some of the resulting RNA transcripts are His_FLAG_GFP_P7′. The RNA transcript is treated with DNase to remove DNA that would otherwise form clusters. In order to check that the DNase treatment is effective, a reaction can be performed and analyzed on a gel to prove that the DNase treatment is effective at removing the DNA.

A PhiX DNA library and the DNase treated GFP-P7′ RNA transcripts are hybridized onto different lanes of a flow cell following the standard cluster protocol for template hybridization. For example, lanes 1-4 can contain the PhiX DNA, while lanes 5-8 contain the GFP RNA. Lanes 5 and 6 can contain RNA that is pre-treated with DNase to remove DNA. Lanes 7 and 8 can be RNA that is pre-treated with DNase and treated with RNase on the flowcell as an additional control. The PhiX DNA library can hybridize via P5 or P7 as both sequences and their complements are present in the template. In contrast, the GFP-P7′ RNA templates hybridize to the P7 surface primers only because of their complementarity and the lack of a P5 sequence. First extension can be carried out by using any commercially reverse transcriptase such as Moloney murine leukemia virus (MMLV) reverse transcriptase. Some lanes can be transposed using a transposome complex containing the transposon sequence P5 adaptor sequence. Gaps in the DNA sequence left after the transposition event can be filled in using a strand displacement extension reaction. The transposition event is required in the lanes containing GFP_P7′ RNA to add the P5 adapter to generate a template that can make clusters. Isothermal cluster amplification can be carried out as standard.

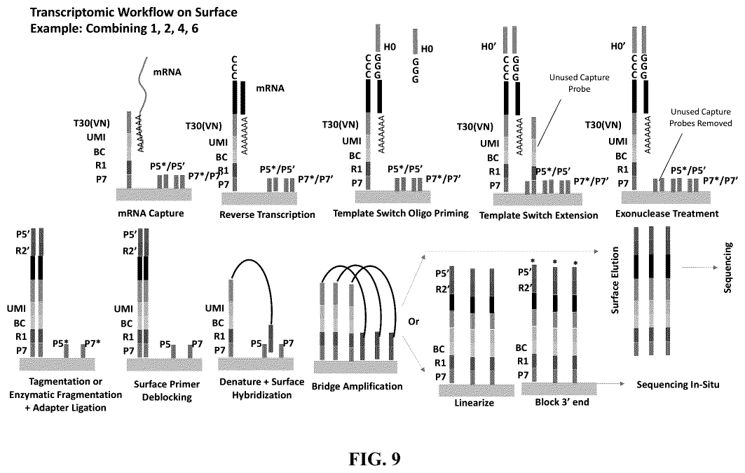

The nucleic acid strand synthesized from the first extension can be sequenced for analysis of the biological samples described herein. In some cases, the strand synthesized from the first extension can be sequenced to obtain transcriptome data. In other instances, the strand synthesized from the first extension can serve as a template for a second extension or second strand synthesis to generate a second strand of nucleic acid. The second strand can be sequenced to obtain transcriptome data. The first strand, second strand, or the amplification of the cDNA molecule can also be obtained by a combinatorial approach where the one or more of the methods described herein can be utilized in conjunction to obtain transcriptome data. FIG. 9 provides a non-limiting example of such combination, where combinatorial uses of the improvements described in FIG. 2 (Library Construction on Surface), FIG. 3 (3′ blocking of surface primers for avoiding unwanted hybridization and extension), FIG. 5 (use of TdT for blocking 3′ end of cDNA), and FIG. 7 (use of exonuclease treatment to remove unwanted capture probes) to improve upon the sequencing techniques currently available.

### Example 2

### RNA Sequencing of a Biological Sample Obtained from Human

A flow cell with multiple lanes can be prepared comprising primers capable of hybridizing to RNA molecules comprising a polyA tail. For example, lane 1 can be grafted with a standard oligonucleotide (oligos) mix only comprising P5 and P7 oligonucleotides, while lanes 2-8 are grafted with standard mix (P5 and P7 oligos) plus the capture oligo (i.e., the primer comprising a polyT sequence for binding to RNA molecules comprising a polyA tail). After primer grafting, the flow cell can be stored in 4° C. until used. In this example, 5 μM of PhiX control library samples can be prepared and added lanes 1 and 2 to the flow cell for hybridization. For each of lane 3-8, 400 ng of RNA sample is prepared and added to the flow cell for hybridization. Lanes 3-6 can contain human RNA such as a biological sample obtained from a subject. Lanes 7 and 8 can contain universal human reference (UHR) RNA. After template hybridization, wash buffer can be administered through the flow cell for removal of unhybridized template. Hybridized templates can be extended using AMV-RT (NEB, Ipswich, Mass.) in all lanes, which produced DNA:RNA complexes.

Lanes 3-8 can be contacted with a transposome complex, while lanes 1 and 2 are contacted with equivalent volume of only wash buffer. Transposome complex mixes of two different concentrations are prepared. The mix for lanes 3, 5 and 7 can be prepared with 1.25 μl of transposome complex, 100 μl of buffer and 400 μl of water. The mix for lanes 4, 6 and 8 can be prepared with 0.625 μl of transposome complex, 100 μl of buffer and 400 μl of water. 95 μl of transposome complex mixes are added to lanes 3-8 of the flow cell for tagmentation. To remove the transposase after tagmentation, chaotropic buffer is added to lanes 3-8 of the flow cell and incubated for 2 minutes. The lanes of the flow cell are then washed twice. After washing, Bst enzyme is used for strand displacement extension of tagmented DNA:RNA complexes to remove the non-transferred strand of the transposon and make the DNA strand of the DNA:RNA complexes full length for clustering. The RNA strands are removed and clusters are then generated using isothermal amplification. The clusters are then sequenced.

The sequencing results are compared to results obtained for standard RNA sequencing of universal human reference RNA, which is carried out according to standard sequencing methods using standard sequencing reagents. The results can show normal alignment distribution for the RNA samples sequenced using the method provided herein. The results can show higher repeat masked clusters likely due to higher numbers of polyA sequences and more repeats in the 3′ UTR regions of the RNA samples analyzed by the tagmentation method. The usable reads can be about 10% lower than for the standard RNA sequencing protocol again likely due to more repeats in the RNA that can be analyzed. The amount of ribosomal RNA can be low as would be expected since mRNA is isolated and sequenced in the tagmentation method provided herein. The mitochondrial RNA is within normal limits.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

### TERMS and DEFINITIONS

“Amplicon” means the product of a polynucleotide amplification reaction; that is, a clonal population of polynucleotides, which may be single stranded or double stranded, which are replicated from one or more starting sequences. “Amplifying” means producing an amplicon by carrying out an amplification reaction. The one or more starting sequences may be one or more copies of the same sequence, or they may be a mixture of different sequences. In some embodiments, amplicons are formed by the amplification of a single starting sequence, so that the amplicon is a clonal population of the starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In regard to amplification reactions, a “reaction mixture” means a solution containing all the necessary reactants for performing a reaction, which may include, but not be limited to, buffering agents to maintain pH at a selected level during a reaction, salts, co-factors, scavengers, and the like. Of special interest are solid phase amplification techniques in which starting sequences are amplified to produce surface-bound copies, such as, bridge amplification or the like, e.g. U.S. Pat. Nos. 6,090,592; 6,060,288; 6,787,308; 9,057,097; 9,169,513; 9,476,080; 9,476,080; Adessi et al, Nucleic Acids Research, 28(20): e87 (2000); and the like; which are incorporated herein by reference.

“Barcode” means a molecular label or identifier. In some embodiments, a barcode is a molecule attached to an analyte or is a segment of an analyte (for example, in the case of polynucleotide barcodes and polynucleotide analytes) which may be used to identify the analyte. In some embodiments, a barcode (referred to herein as a “spatial barcode”) may be attached to a surface to identify a location on the surface. In some embodiments, populations of identical spatial barcodes may be disposed within a particular area on a surface. In some embodiments, there may be a one-to-one correspondence between different spatial barcodes and different areas on a surface; that is, each different area has a different and unique barcode. In some embodiments, the identity of a spatial barcode is determinable, for example, by sequencing whenever a spatial barcode is a polynucleotide. In some embodiments, a spatial barcode is an oligonucleotide. In some embodiments, a barcode comprises a random sequence oligonucleotide. A random sequence oligonucleotide is typically synthesized by a “split and mix” synthesis techniques, for example, as described in the following references that are incorporated herein by reference: Church, U.S. Pat. No. 4,942,124; Godron et al, International patent publication WO2020/120442; Seelig et al, U.S. patent publication 2016/0138086; and the like. Sometimes a random oligonucleotide is represented as “NNN . . . N.” In some embodiments, the term “barcode” includes composite barcodes; that is, an oligonucleotide segment that comprises sub-segments that identify different objects. For example, a first segment of a composite barcode may identify a particular area of a surface and a second segment of a composite barcode may identify a particular molecule (a so-called “unique molecular identifier” or UMI).

“Cells” refers to biological cells that may be assayed by methods and systems of the invention comprise, but are not limited to, vertebrate, non-vertebrate, eukaryotic, mammalian, microbial, protozoan, prokaryotic, bacterial, insect, or fungal cells. In some embodiments, mammalian cells are assayed by methods and systems of the invention. In particular, any mammalian cell which may be, or has been, genetically altered for use in a medical, industrial, environmental, or remedial process, may be analyzed by methods and systems of the invention. In some embodiments, “cells” as used herein comprise genetically modified mammalian cells. In some embodiments, “cells” comprise stem cells. In some embodiments, “cells” refer to cells modified by CRISPR Cas9 techniques. In some embodiments, “cells” refer to cells of the immune system including, but not limited to, cytotoxic T lymphocytes, regulatory T cells, CD4+ T cells, CD8+ T cells, natural killer cells, antigen-presenting cells, or dendritic cells. Of special interest are cytotoxic T lymphocytes engineered for therapeutic applications, such as cancer therapy.

“Cluster” means an amplicon or clonal population of a single polynucleotide amplified by a surface amplification technique, such as bridge PCR. In some embodiments, the term “cluster” includes amplicons produced by rolling circle amplification.

“Hydrogel” means a gel comprising a crosslinked hydrophilic polymer network with the ability to absorb and retain large amounts of water (for example, 60 to 90 percent water, or 70 to 80 percent) without dissolution due to the establishment of physical or chemical bonds between the polymeric chains, which may be covalent, ionic or hydrogen bonds. Hydrogels exhibit high permeability to the oxygen and nutrients, making them attractive materials for cell encapsulation and culturing applications. Hydrogels may comprise natural or synthetic polymers and may be reversible (i.e. degradable or depolymerizable) or irreversible. Synthetic hydrogel polymers may include polyethylene glycol (PEG), poly(2-hydroxyethyl methacrylate) and poly(vinyl alcohol). Natural hydrogel polymers include alginate, hyaluronic acid and collagen. The following reference describe hydrogels and their biomedical uses: Drury et al, Biomaterials, 24: 4337-4351 (2003); Garagorri et al, Acta Biomatter, 4(5): 1139-1147 (2008); Caliari et al, Nature Methods, 13(5): 405-414 (2016); Bowman et al, U.S. Pat. No. 9,631,092; Koh et al, Langmuir, 18(7): 2459-2462 (2002).

“On demand” means an operation may be directed to individual, discrete, selected locations (e.g. a spatial location of polymer precursor solution; or a selected polymer matrix chamber). Such selection may be based on manual observation of optical signals or data collected by a detector, or such selection may be based on a computer algorithm operating on optical signals or data collected by a detector. Manual observation of optical signals or data collected by a detector can include either real-time detection or detection at a time period prior to modulating a unit of energy to polymerize polymer precursors or degrading a chamber. For example, a subset of chambers (all formed with photo-degradable polymer matrix walls) may be pre-selected for releasing and removing their contents based on position information and the values of optical signals from an analytical assay carried out in the chambers. The pre-selected chambers may be photo-degraded by selectively projecting a light beam of appropriate wavelength characteristics (for example, with the spatial energy modulating element) to degrade the polymer matrix walls of the pre-selected chambers. In another example, a plurality of chambers may be observed in real-time (e.g. via fluorescent microscopy) for detection of an analyte of interest and one or more chambers of the plurality of chambers is selected, in real-time, upon detection of the analyte of interest, for degradation.

“Physical photomask” generally refers to a physical structure having a plurality of apertures or holes through which light may be projected. Physical photomasks can be used to create hydrogel matrices as described herein by causing the polymer precursor solution to polymerize and forming three-dimensional structures that correspond to the pattern on the photomask. A physical photomask can be patterned with a specific layout or geometric pattern. A physical photomask may be adhered to the upper surface of a flow cell.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, bridge PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” or “quantitative PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“tagman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference.

“Polymer matrix” generally refers to a phase material (e.g. continuous phase material) that comprises at least one polymer. In some embodiments, the polymer matrix refers to the at least one polymer as well as the interstitial space not occupied by the polymer. A polymer matrix may be composed of one or more types of polymers. A polymer matrix may include linear, branched, and crosslinked polymer units. A polymer matrix may also contain non-polymeric species intercalated within its interstitial spaces not occupied by polymer chains. The intercalated species may be solid, liquid, or gaseous species. For example, the term “polymer matrix” may encompass desiccated hydrogels, hydrated hydrogels, and hydrogels containing glass fibers. A polymer matrix may comprise a polymer precursor, which generally refers to one or more molecules that upon activation can trigger or initiate a polymeric reaction. A polymer precursor can be activated by electrochemical energy, photochemical energy, a photon, magnetic energy, or any other suitable energy. As used herein, the term “polymer precursor” includes monomers (that are polymerized to produce a polymer matrix) and crosslinking compounds, which may include photo-initiators, other compounds necessary or useful for generating polymer matrices, especially polymer matrices that are hydrogels.

“Polynucleotide” and “oligonucleotide” are used interchangeably and each means a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or intemucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as examples only.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

DNA:RNA duplex can refer to the complex formed when captured RNA and reverse transcribed complementary DNA (cDNA) from the captured RNA. In some cases, the RNA of the DNA:RNA duplex can be denatured or washed off and the cDNA can be subjected to a second strand synthesis reaction to generate double stranded DNA (dsDNA).

Transposon or transposase can refer to a small sequence of nucleotides that have the ability to pretty-much arbitrarily move locations (translocate).

The term “sample,” as used herein, generally refers to a chemical or biological sample containing a biological component. The biological component may comprise a cell, a nucleic acid, a microbiome, a protein, a combination of cells, a metabolite, a combination thereof, or any other suitable component of a biological sample. For example, a sample can be a biological sample including one or more cells. For another example, a sample can be a biological sample including one or more nucleic acids. The biological sample can be obtained (e.g., extracted or isolated) from or include blood (e.g., whole blood), plasma, serum, urine, saliva, mucosal excretions, sputum, stool, and tears. The biological sample can be a fluid or tissue sample (e.g., skin sample). In some instances, the sample may be derived from a homogenized tissue sample (e.g., brain homogenate, liver homogenate, or kidney homogenate). In certain embodiments, the sample may include a specific type of cell (e.g., a neuronal cell, muscle cell, liver cell, or kidney cell,). The sample may comprise or be acquired from a diseased cell or tissue (e.g., a tumor cell or a necrotic cell), In some embodiments, the sample may include or may be from a disease-associated inclusion (e.g., a plaque, a biofilm, a tumor, or a non-cancerous growth). In certain embodiments, the sample may include or may be obtained from a cell-free bodily fluid, such as whole blood, saliva, or urine. In various embodiments, the sample can include circulating tumor cells. In some cases, the sample may include or may be an environmental sample (e.g., soil, waste, or ambient air), industrial sample (e.g., samples from any industrial processes), or a food sample (e.g., dairy product, vegetable product, or meat product). The sample may be processed prior to loading into a microfluidic device. For example, the sample may be processed to purify a certain cell type or nucleic acid and/or to include reagents.

As used herein, “tagmentation” refers to the modification of DNA by a transposome complex comprising transposase enzyme complexed with adaptors comprising transposon end sequence. Tagmentation results in the simultaneous fragmentation of the DNA and ligation of the adaptors to the 5′ ends of both strands of duplex fragments. Following a purification step to remove the transposase enzyme, additional sequences can be added to the ends of the adapted fragments, for example by PCR, ligation, or any other suitable methodology. A “transposome” is comprised of at least a transposase enzyme and a transposase recognition site. In some such systems, termed “transposomes”, the transposase can form a functional complex with a transposon recognition site that is capable of catalyzing a transposition reaction. The transposase or integrase may bind to the transposase recognition site and insert the transposase recognition site into a target nucleic acid in a process sometimes termed “tagmentation”. In some such insertion events, one strand of the transposase recognition site may be transferred into the target nucleic acid. In standard sample preparation methods, each template contains an adaptor at either end of the insert and often a number of steps are required to both modify the DNA or RNA and to purify the desired products of the modification reactions. These steps are performed in solution prior to the addition of the adapted fragments to a flow cell where they are coupled to the surface by a primer extension reaction that copies the hybridized fragment onto the end of a primer covalently attached to the surface.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for preparing a set of complementary deoxynucleic acid (cDNA) molecules or derivatives thereof from one or more nucleic acid molecules, the method comprising:
(a) contacting said one or more nucleic acid molecule capture probes on a solid support with said one or more nucleic acid molecules to yield one or more captured nucleic acid molecules, wherein said solid support comprises a plurality of surface primer probes;
(b) synthesizing a cDNA molecule from said captured nucleic acid molecule or a derivative, wherein said cDNA molecule is coupled to a surface primer probe of said plurality of surface primer probes;
(c) inserting an adapter at the 3′ region of said cDNA molecule or a derivative thereof; and
(d) amplifying said cDNA molecule or a derivative thereof to generate said set of cDNA molecules or derivates thereof, wherein said set of cDNA molecules or derivates thereof is coupled to a surface primer probe of said plurality of surface primer probes.

2. The method of claim 1, wherein said adapter comprises a sequence configured to permit initiation of a sequencing reaction on a cDNA molecule of said set of cDNA molecules or derivatives thereof.

3. (canceled)

4. The method of claim 1, comprising, following (a), contacting said solid support with a moiety configured to inactivate at least a subset of said one or more nucleic acid molecule capture probes.

5. The method of claim 4, wherein said subset of said one or more nucleic acid molecule capture probes comprise one or more nucleic acid molecule capture probes that did not capture a nucleic acid molecule.

6. The method of claim 4, wherein said moiety configured to inactivate at least said subset of said one or more nucleic acid molecule capture probes comprises an exonuclease.

7. The method of claim 1, wherein said synthesizing comprises performing one or more second strand synthesis reactions comprising said cDNA molecule or a derivative thereof.

8. The method of claim 1, comprising, prior to (c), amplifying said cDNA molecule or a derivative thereof, wherein said amplifying comprises in-solution primer sequences.

9. (canceled)

10. The method of claim 1, further comprising, prior to (c), fragmentation of said cDNA molecule or a derivative thereof, thereby obtaining one or more cDNA fragments of said cDNA molecule, and wherein said inserting of said adapter in (c) comprises ligating said adapter to a cDNA fragment of said one or more cDNA fragments.

11. The method of claim 1, wherein said inserting of said adapter at said 3′ region of said cDNA molecule or a derivative thereof in (c) comprises one of single-strand ligation, tagmentation, and double-strand ligation.

12. The method of claim 1, wherein at least a subset of said plurality of surface primer probes comprises a blocking agent that blocks an extension reaction on said at least said subset of said plurality of surface primer probes, and wherein the method further comprises prior to (d), subjecting said blocking agent to a reaction that unblocks said at least said subset of said plurality of surface primed probes to permit said extension reaction.

13. (canceled)

14. (canceled)

15. (canceled)

16. The method of claim 1, comprising, following (d), cleaving or linearizing at least a subset of said set of cDNA molecules or derivatives thereof.

17. The method of claim 1, following (d), blocking the 3′ end of said subset of said set of DNA molecules or derivatives thereof.

18. (canceled)

19. (canceled)

20. (canceled)

21. The method of claim 1, comprising sequencing said at least said subset of said cDNA molecules or derivatives thereof in situ on said solid support.

22. The method of claim 1, comprising eluting at least a subset of said set of cDNA molecules or derivatives thereof from said solid support.

23. (canceled)

24. (canceled)

25. (canceled)

26. The method of claim 1, wherein a sequence of a nucleic acid molecule capture probe of said one or more nucleic acid molecule capture probes is configured to couple to said one or more nucleic acid molecules, and wherein said sequence of said nucleic acid molecule capture probe comprises a poly-T sequence, a randomer, a sequence complementary to at least a subset of said one or more nucleic acid molecules, or any combination thereof.

27. The method of claim 1, wherein said solid support is a fluidic channel.

28. The method of claim 27, wherein said fluidic channel is a flow cell.

29. The method of claim 1, wherein said solid support is not a bead.

30. The method of claim 1, wherein said one or more nucleic acid molecule capture probes comprise one or more tags, wherein a tag comprises a cell-specific or spatial location-specific identifier sequence and optionally a unique molecular identifier (UMI) sequence.

31. The method of claim 1, wherein said amplifying comprises solid-supported amplification.

32. (canceled)

33. The method of claim 1, wherein said one or more nucleic acid molecules are derived from a single cell or biological tissue.

34.-173. (canceled)

174. The method of claim 1, wherein said solid support comprises a hydrogel chamber disposed thereon, wherein the hydrogel chamber comprises one or more polymer matrix walls.

175. The method of claim 174, wherein said one or more polymer matrix walls extend from said solid support to a top surface opposite of said solid support, thereby forming an interior of said hydrogel chamber.

176. The method of claim 175, wherein said one or more nucleic acids are derived from a single cell, and wherein prior to (a), said interior of said hydrogel chamber comprises said single cell.

177. The method of claim 176, wherein prior to (a), said one or more nucleic acid molecules are released from said single cell.

178. The method of claim of claim 176, wherein prior to (a), a lysis reagent is introduced to said fluidic device, thereby causing said one or more nucleic acid molecules to be released from said single cell.

179. The method of claim 175, wherein any of (a)-(d), or any combination thereof, occur in said interior of said hydrogel chamber.

180. The method of claim 174, further comprising degrading the hydrogel chamber subsequent to (a) and prior to (b).
